SEQUENCE LISTING (142 sequences)

SEQ ID NO: 1 - DNA, 6410 bp, Oryza sativa
  promoter (1)..(6410): promoter of the gene 12(Oryza sativa)
tacaaaggagtccacatcaaccctccagacaaacgtgcgcactaaagttttgctgtaagcttggaaagtaaactttgtactccatccagcctattaatacttgttgtagtgcactttgtccagctgtgttgtcaaagttcatttcttgtgttttcccccaaggtagcatatctgaatggtgactaacaggttaaccgtagagccaggattagctgcctgacttgatgagaatatgtcgtctcacgttccttaaatactgatgtgaaacatcatgcattttgcaggctgttgagaagaggagaggaagaaagaagagccttttcttcaagtaggtttggctcctataccgtggtgtataatttgtattcgtgattggctggatgtatgtaattaagcgggtgataggtttggcatattctttcggctgtatcttctgctgtgaaggaccataggcgatttaaacacaagggccccaggtgatgggagttttcttcttttttttccttcttccttttagaaacatgagttttgtcgttgcaagggatggaagcgtgccgctgattagaattgaagctaatttatgctaatagaatggaaaagataaataggcaagtcctaggatgctctggaaatattccctgcatcgtagggtagtgacgatcatcagttcacgggtgacgtccctgcctgctggttgcttccaacgagcttgcaattttgaagatgacgcatagcagtgcttgaacatgtatacaagtataactgatgtatcatctgctgtgtaattaattgagacgaagtagacttgtttgaatttggagatagcaggagtagataagataacaagagcatcttagttattccaacgtgtactgtactgctggaagaaaacacttgcattattaggcagttgtacgtagaagaaagaatctgggttgcagttgtgtctacatcctatgaacgttctgtggatggtacagtgacattgaggcgggcaatgtccacaccacacttgccaaacagaatgtgaaggcgtttggcataaaggtcaactggaactttcaattacccgtagtaattgaaggagataacgtggcatctacacgcatgtggtatgacgtgtggctgctaacatcagcagcagtgttcatgctgcttgctgttcatgaattaattagctgtccatccctcctataattgcattcttttttactacccctccttcgttacctatagatggaattaaaacagacaaaattatgattaattagttgagaaaaaagaagtacgtgaagaaattaattgggatggttgtgattgatttagattagactatatggaaaagttgttctatgtttgggacggggagagtggtcaataaatttctctccacgggtacaggtcaggcaaagggtatgaacttggagacagtatgcatcaggactgcttatcaaggaattcttcagtaggtcctccctgagacaaatcaataaaggcgagcgattagactttctaatttgactattatttctagagctaaaccacatacttcatccatttcaaaatataaggagttatagatttccatccatccaggggcggatctagaaaaaaatagtagcgggggctaaataaactacatcaatataaatcgaacctccagtcctcacatcctatagatctatggaaaaaaatttagtgggggcttaggggggtctccattgtcccgggggcggtagtggggtctcaagacccctccgcccccattgtggatccgccccagcatccatcctaaaatataaggtgttatggatattttgggatggaggagaagtatcattgtcgatcgatcaccggctgctgctacccaatcacatgcattttcccccttcactgccatattatgcaacaggatcgatatttcaaaccctcattttaatttgtacgatcttcaaattaatttcgctggagactatggtggagagctactactcatacagtcatactactagaatgcaacagtaaatgtattaatagtggcaagacaaagagaagaatgggctacagtgaaacaaataatccacaacaataatcaataatcgtgtgaataatagtactccccacgtcttcaaaagactgtagttttaactgaagaatttgtccaacaaaaaacaaatgtatttttagagtgaggtctattagacatctttaaggtccctttgatttatagaaaacataaaaaatttggagaatttcaattttatagaaaagttttctataaaggtatttgaaaaaaaaattgaatcaagagattttgaagaaaacttatcaagagctctaatctcttggaaaattttctttgaatccttattcgatttctatatttttcctatgcttcaatcaaacgagtattcatgtgttttttctgtgttttacaatcatctgtttttgctattgtattgctgtcagaatcatgtgtttttcttatgcctccgtttttccatttatgcaattcaaagggagcaattagtattttgttcttgttttctctccgtaataataaattgcacggcagtctttaaaactaactttttttaagggacataggtacggatggagagcattacgatggatacatgcatgaaccgcgagagtgatacaaactggctagtacagtacaaacagtagctacctagtcgttgtggatacatatgcatgcatgcatatacatgtacatgtatgtatgtatgtacgatcaggacaggcatatatatctggatctgcttaattggatatattattcgtagccttttgttttgtttttctagctacagtgtacactaatagtgtacccttccgtctaagaaaatctaatcctaagtacaaacgtgaatatatatgtgttcagatttatagctaacgttttttttttttacggagtagcagtacttggactgatttcaccgctcgtaattatatgcaggtccatgtcgatggatcctctctcagatcgtctgcgccacacaggacaaaccagtagtatatatgcatcctgaccatacattgtatacacatgtacatacatgtaagggtaattaattaagtctaattaactattgcttccataaaaaaatattacttcctatatcccaaaataaatacagtcgtatactttaaatttggacaacacggctgcatattgttcaaattttgggatgtacaccctccgtccaaaaaaaaagacaaaccctagatttacgtgtccaacgtttgactgtccgtcttatatgaaatttttttataattagtcttttcattgttgttagataataaaacatgattaatactttatgcgtgacttgtctttttaattttttttaaataagacggacggtcaaacattggacacggaaacccagggcttagctttactacatactcctgttctaccaacaatcaattgtctctgtttgtttgagctataacttattttgaattatagaattgacttaattttagaaattactttaacaatttttcatttgctcaaatctcctcaaagtatgttttgttctggtccccgcccggcctgtatatatggtggaccaaaacgacatgtgtatatatatcgtcctgtactatacatatctgccaatctatagctaaagctcgactagtgtgactggacagctagaacctgcaatattccagtctcttgggtatgtatgtttgtcgtatatgcagaagtactattgcagtatggagtatattctttccgtcatgcaatttttgacgttttactagattatttaccatatattaaggtttgaaaaggaatactatagtgatcttattaaataatatatgattgaaacttaaaaataaaagggtgattgagaagagtaagtagaaatttggaccaaaaatgattaatgcatgggacaaatattagtaatactttattgtctaaattttttatgataaattttaaattttagagagagatagtaactagtagtattttgggacgggacgtgtgtatatgttaaactgaataaagtttgggctgtggctaacgtacctgtggatgtaccaagtgtattgtacgtatatgtttgttggtttgtgcagctgtgctagctagctgctactacagcctcttgtgccttgtgtacatttgtctcctatctacagtatctgtttcatatccatagatattagatgtatgagtatccatccatcttgtaggctgctagccagtcagcagttacaaccacaacatcaatagatgcatgtgcaacacatacacacaccttacgtcacccttgctgccgtcaccagcaattaacgttagttaatgctcaatgccagcacacgctttagttgacacactatcacaaccactccctactcctacgcgcgcgtacgagtaacatgcatttagctggaatattgttcaacattcaattcaaggcgatctatatatcatcaactgtttatgttagcgctatcaagtttaataatactgtatatatagcgctacaagaacgtactactctccctttgcaaaatttgataacctataatagaagattagacctattttagaactacgaatttaaatagatatgaataatttttaaattcgtagttataaaatggatctattctctcattttagttcgttatatattgaaacgaagagagtagttttagaggttatataaaaggttagtattagaactaattagcggcaacgttcggatgtgctggcgcctggctgccatagtagagcagcaatgcaagcttgggtcatgcagcatgcagctagctgtggctgctacgatcgtatctagcggcaccgaattaacactccctaacatttagtacgtacgacgctagttataacactgttttcatttttatccttcccttttaatttctgtacttaacgcaactcttttggagtacgtgcaatatatatgtactggttcttgtatgaaaaccgacaccatttatgtcttttctttagttaatgaaataaaatctcggtctttactcaatgagatgtaatcaattagtataaatttgcgtagacaaaattgcacatgaacaagtcataacaagcacaccgaaaatatgaacggggtagtatgtcatcagtacagaaaatagttgagagattagcatgggagtcagtgtaatactagatggaatagagggcgaaatgaatgagatccagaaagagcagactctagctcctgcaaacactgtaagagtacggcctggtttagttcctaactttttttaaaaaacttttaacttttttatcccattaaaatttttctacacacaaacttctaattttttctatcacatcatatcaattttaatcaaatttttaattttggcgtgaaccacaccctacgcttgtggaccctggctatcgcaagtgcatttaatgcagggtcccagcagcaccacatgaatttgcagccgtcctggtggtgtagtggggctatagctagctgtaggtacgtgtacgtgtaaatactatagtatacagggatcatatattagttacgtgctctaaaatatgtataggctaccttgctatcactaatgttttagaacacatatatattgtgttgctgtgtttggtcatatataatatactacccctaccctagaatataaagacattacatagtataatgaatctagatatgattatatctagattcgttgtgctatgtaatgtcccatccgtgctgtatagtttgggtttttttttacggaggaagtatatggttagacacgattactaagaaagtggtaaaattaaatgaaaaaaatgttgtaactggataagaagtgtaggcatgtgagaaaatttaatgatggatattatgattggttgggatcagaatgttggtgaaaaagttattatattttggaacaaactttgatagctaaaaattattatattttaagatagaggaaacattgattgtatgcatgctatatatatatatagaattatacggaataaatacaaacggaagggcctagctagagctaccagctgcgtgcaaatttattccgtataatttggaaggatatatcttgcttcattttgctattagcttgctttggctatcttaattaatatttaaaaaacgaatggtgctgatttgctctctcatctttataaatatgagcactcgtctctctgtacatagagagagtgagagataacgcgcgggcgctagctagctagctcttaatgtgatatgtatacacaatttttcctttatctgtcaaggaatgggtaaatgcatggttgagagaattaattgagtttcagttatagatatatagctggctgagacgaccagttagccatcgtc

SEQ ID NO: 2 - DNA, 540 bp, Oryza sativa
  CDS (1)..(540): Hd3a cDNA(Kasalath)
atggccggaagtggcagggacagggaccctcttgtggttggtagggttgtgggtgatgtgctggacgcgttcgtccggagcaccaacctcaaggtcacctatggctccaagaccgtgtccaatggctgcgagctcaagccgtccatggtcacccaccagcctagggtcgaggtcggcggcaatgacatgaggacattctacacccttgtgatggtagacccagatgcaccaagcccaagtgaccctaaccttagggagtatctacattggttggtcactgatattcctggtactactgcagcgtcatttgggcaagaggtgatgtgctacgagagcccaaggccaaccatggggatccaccggctggtgttcgtgctgttccagcagctggggcgtcagacagtgtacgcgcccgggtggcgtcagaacttcaacaccaaggacttcgccgagctctacaacctcggctcgccggtcgccgccgtctacttcaactgccagcgcgaggccggctccggcggcaggagggtctacaactag

SEQ ID NO: 3 - protein, 179 aa, Oryza sativa
MAGSGRDRDPLVVGRVVGDVLDAFVRSTNLKVTYGSKTVSNGCELKPSMVTHQPRVEVGGNDMRTFYTLVMVDPDAPSPSDPNLREYLHWLVTDIPGTTAASFGQEVMCYESPRPTMGIHRLVFVLFQQLGRQTVYAPGWRQNFNTKDFAELYNLGSPVAAVYFNCQREAGSGGRRVYN

SEQ ID NO: 4 - DNA, 540 bp, Oryza sativa
  CDS (1)..(540): Hd3a cDNA(Nippon-bare)
atggccggaagtggcagggacagggaccctcttgtggttggtagggttgtgggtgatgtgctggacgcgttcgtccggagcaccaacctcaaggtcacctatggctccaagaccgtgtccaatggctgcgagctcaagccgtccatggtcacccaccagcctagggtcgaggtcggcggcaatgacatgaggacattctacacccttgtgatggtagacccagatgcaccaagcccaagtgaccctaaccttagggagtatctacattggttggtcactgatattcctggtactactgcagcgtcatttgggcaagaggtgatgtgctacgagagcccaaggccaaccatggggatccaccggctggtgttcgtgctgttccagcagctggggcgtcagacagtgtacgcgcccgggtggcgtcagaacttcaacaccaaggacttcgccgagctctacaacctcggctcgccggtcgccgccgtctacttcaactgccagcgcgaggcaggctccggcggcaggagggtctacccctag

SEQ ID NO: 5 - protein, 179 aa, Oryza sativa
MAGSGRDRDPLVVGRVVGDVLDAFVRSTNLKVTYGSKTVSNGCELKPSMVTHQPRVEVGGNDMRTFYTLVMVDPDAPSPSDPNLREYLHWLVTDIPGTTAASFGQEVMCYESPRPTMGIHRLVFVLFQQLGRQTVYAPGWRQNFNTKDFAELYNLGSPVAAVYFNCQREAGSGGRRVYP

SEQ ID NO: 6 - DNA, 774 bp, Oryza sativa
  CDS (1)..(774): Ghd7 cDNA
atgtcgatgggaccagcagccggagaaggatgtggcctgtgcggcgccgacggtggcggctgttgctcccgccatcgccacgatgatgatggattccccttcgtcttcccgccgagtgcgtgccaggggatcggcgccccggcgccaccggtgcacgagttccagttcttcggcaacgacggcggcggcgacgacggcgagagcgtggcctggctgttcgatgactacccgccgccgtcgcccgttgctgccgccgccgggatgcatcatcggcagccgccgtacgacggcgtcgtggcgccgccgtcgctgttcaggaggaacaccggcgccggcgggctcacgttcgacgtctccctcggcgaacggcccgacctggacgccgggctcggcctcggcggcggcggcggccggcacgccgaggccgcggccagcgccaccatcatgtcatattgtgggagcacgttcactgacgcagcgagctcgatgcccaaggagatggtggccgccatggccgatgatggggagagcttgaacccaaacacggtggttggcgcaatggtggagagggaggccaagctgatgaggtacaaggagaagaggaagaagaggtgctacgagaagcaaatccggtacgcgtccagaaaagcctatgccgagatgaggccccgagtgagaggtcgcttcgccaaagaacctgatcaggaagctgtcgcaccgccatccacctatgtcgatcctagtaggcttgagcttggacaatggttcagatag

SEQ ID NO: 7 - protein, 257 aa, Oryza sativa
MSMGPAAGEGCGLCGADGGGCCSRHRHDDDGFPFVFPPSACQGIGAPAPPVHEFQFFGNDGGGDDGESVAWLFDDYPPPSPVAAAAGMHHRQPPYDGVVAPPSLFRRNTGAGGLTFDVSLGERPDLDAGLGLGGGGGRHAEAAASATIMSYCGSTFTDAASSMPKEMVAAMADDGESLNPNTVVGAMVEREAKLMRYKEKRKKRCYEKQIRYASRKAYAEMRPRVRGRFAKEPDQEAVAPPSTYVDPSRLELGQWFR

SEQ ID NO: 8 - DNA, 53 bp, Artificial
  Artificially synthesized primer sequence
cgggatccatgtatccatacgatgttccagattatgctgtcggcgccggttgg

SEQ ID NO: 9 - DNA, 51 bp, Artificial
  Artificially synthesized primer sequence
ggcgcctcctttttcaaattgaggatgagaccaaccggcgccgacagcata

SEQ ID NO: 10 - DNA, 25 bp, Artificial
  Artificially synthesized primer sequence
atgtcgatgggaccagcagccggag

SEQ ID NO: 11 - DNA, 29 bp, Artificial
  Artificially synthesized primer sequence
cggaattctatctgaaccattgtccaagc

SEQ ID NO: 12 - DNA, 30 bp, Artificial
  Artificially synthesized primer sequence
aaaagagggggattaatggccggaagtggc

SEQ ID NO: 13 - DNA, 30 bp, Artificial
  Artificially synthesized primer sequence
ggaaattcgagctcggtaccctagttgtag

SEQ ID NO: 14 - DNA, 27 bp, Artificial
  Artificially synthesized primer sequence
gaattccaagcaacgaactgcgagtga

SEQ ID NO: 15 - DNA, 27 bp, Artificial
  Artificially synthesized primer sequence
taatccccctctttttcaaagaacaag

SEQ ID NO: 16 - DNA, 30 bp, Artificial
  Artificially synthesized primer sequence
cataagggcctctagagaattccaagcaac

SEQ ID NO: 17 - DNA, 20 bp, Artificial
  Artificially synthesized primer sequence
aagctttgcagcgtgacccg

SEQ ID NO: 18 - DNA, 21 bp, Artificial
  Artificially synthesized primer sequence
aagcttgatctagtaacatag

SEQ ID NO: 19 - DNA, 17134 bp, Artificial
  pRiceFOX/UbiGhd7/GateHd3a
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatcgatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcttattgccccccgggatccccctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaagggaattcctgcagcccgggggatccactagaaccggtgacgtcaccatgggaagctttgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaagcggggatcctctagagtcgacctgcaggcatgcaagctagcttacaagtttgtacaaaaaagcaggctttaaaggaaccaattcagtcgactggatccatgtatccatacgatgttccagattatgctgtcggcgccggttggtctcatcctcaatttgaaaaaggaggcgccatgtcgatgggaccagcagccggagaaggatgtggcctgtgcggcgccgacggtggcggctgttgctcccgccatcgccacgatgatgatggattccccttcgtcttcccgccgagtgcgtgccaggggatcggcgccccggcgccaccggtgcacgagttccagttcttcggcaacgacggcggcggcgacgacggcgagagcgtggcctggctgttcgatgactacccgccgccgtcgcccgttgctgccgccgccgggatgcatcatcggcagccgccgtacgacggcgtcgtggcgccgccgtcgctgttcaggaggaacaccggcgccggcgggctcacgttcgacgtctccctcggcgaacggcccgacctggacgccgggctcggcctcggcggcggcggcggccggcacgccgaggccgcggccagcgccaccatcatgtcatattgtgggagcacgttcactgacgcagcgagctcgatgcccaaggagatggtggccgccatggccgatgatggggagagcttgaacccaaacacggtggttggcgcaatggtggagagggaggccaagctgatgaggtacaaggagaagaggaagaagaggtgctacgagaagcaaatccggtacgcgtccagaaaagcctatgccgagatgaggccccgagtgagaggtcgcttcgccaaagaacctgatcaggaagctgtcgcaccgccatccacctatgtcgatcctagtaggcttgagcttggacaatggttcagatagaattcgcggccgcactcgagatatctagacccagctttcttgtacaaagtggtgatactagtcccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcaagcttcgacctcgagacaagtttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgccgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagtggtctcgagggccataagggcctctagaatggccggaagtggcagggacagggaccctcttgtggttggtagggttgtgggtgatgtgctggacgcgttcgtccggagcaccaacctcaaggtcacctatggctccaagaccgtgtccaatggctgcgagctcaagccgtccatggtcacccaccagcctagggtcgaggtcggcggcaatgacatgaggacattctacacccttgtgatggtagacccagatgcaccaagcccaagtgaccctaaccttagggagtatctacattggttggtcactgatattcctggtactactgcagcgtcatttgggcaagaggtgatgtgctacgagagcccaaggccaaccatggggatccaccggctggtgttcgtgctgttccagcagctggggcgtcagacagtgtacgcgcccgggtggcgtcagaacttcaacaccaaggacttcgccgagctctacaacctcggctcgccggtcgccgccgtctacttcaactgccagcgcgaggccggctccggcggcaggagggtctacaactagggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggccttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

SEQ ID NO: 20 - DNA, 17235 bp, Artificial
  pRiceFOX/UbiGhd7/GateAdh5′UTRHd3a
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatcgatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcttattgccccccgggatccccctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaagggaattcctgcagcccgggggatccactagaaccggtgacgtcaccatgggaagctttgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaagcggggatcctctagagtcgacctgcaggcatgcaagctagcttacaagtttgtacaaaaaagcaggctttaaaggaaccaattcagtcgactggatccatgtatccatacgatgttccagattatgctgtcggcgccggttggtctcatcctcaatttgaaaaaggaggcgccatgtcgatgggaccagcagccggagaaggatgtggcctgtgcggcgccgacggtggcggctgttgctcccgccatcgccacgatgatgatggattccccttcgtcttcccgccgagtgcgtgccaggggatcggcgccccggcgccaccggtgcacgagttccagttcttcggcaacgacggcggcggcgacgacggcgagagcgtggcctggctgttcgatgactacccgccgccgtcgcccgttgctgccgccgccgggatgcatcatcggcagccgccgtacgacggcgtcgtggcgccgccgtcgctgttcaggaggaacaccggcgccggcgggctcacgttcgacgtctccctcggcgaacggcccgacctggacgccgggctcggcctcggcggcggcggcggccggcacgccgaggccgcggccagcgccaccatcatgtcatattgtgggagcacgttcactgacgcagcgagctcgatgcccaaggagatggtggccgccatggccgatgatggggagagcttgaacccaaacacggtggttggcgcaatggtggagagggaggccaagctgatgaggtacaaggagaagaggaagaagaggtgctacgagaagcaaatccggtacgcgtccagaaaagcctatgccgagatgaggccccgagtgagaggtcgcttcgccaaagaacctgatcaggaagctgtcgcaccgccatccacctatgtcgatcctagtaggcttgagcttggacaatggttcagatagaattcgcggccgcactcgagatatctagacccagctttcttgtacaaagtggtgatactagtcccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcaagcttcgacctcgagacaagtttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgccgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagtggtctcgagggccataagggcctctagagaattccaagcaacgaactgcgagtgattcaagaaaaaagaaaacctgagctttcgatctcttcggagtggtttcttgttctttgaaaaagagggggattaatggccggaagtggcagggacagggaccctcttgtggttggtagggttgtgggtgatgtgctggacgcgttcgtccggagcaccaacctcaaggtcacctatggctccaagaccgtgtccaatggctgcgagctcaagccgtccatggtcacccaccagcctagggtcgaggtcggcggcaatgacatgaggacattctacacccttgtgatggtagacccagatgcaccaagcccaagtgaccctaaccttagggagtatctacattggttggtcactgatattcctggtactactgcagcgtcatttgggcaagaggtgatgtgctacgagagcccaaggccaaccatggggatccaccggctggtgttcgtgctgttccagcagctggggcgtcagacagtgtacgcgcccgggtggcgtcagaacttcaacaccaaggacttcgccgagctctacaacctcggctcgccggtcgccgccgtctacttcaactgccagcgcgaggccggctccggcggcaggagggtctacaactagggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggccttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

SEQ ID NO: 21 - DNA, 21 bp, Artificial
  Artificially synthesized primer sequence
tgcagcgtgacccggtcgtgc

SEQ ID NO: 22 - DNA, 19 bp, Artificial
  Artificially synthesized primer sequence
agtaacaccaaacaacagg

SEQ ID NO: 23 - DNA, 24 bp, Artificial
  Artificially synthesized primer sequence
aagaacatttacataataagcagg

SEQ ID NO: 24 - DNA, 22 bp, Artificial
  Artificially synthesized primer sequence
gggctgctggatcgagctgtgg

SEQ ID NO: 25 - DNA, 21 bp, Artificial
  Artificially synthesized primer sequence
ttctccaaccgtgcgtgtagg

SEQ ID NO: 26 - DNA, 21 bp, Artificial
  Artificially synthesized primer sequence
gagagaagctcaagacacgca

SEQ ID NO: 27 - DNA, 20 bp, Artificial
  Artificially synthesized primer sequence
aagccacttccacgacaggc

SEQ ID NO: 28 - DNA, 22 bp, Artificial
  Artificially synthesized primer sequence
ggcggacacaaggtgtttgtgg

SEQ ID NO: 29 - DNA, 22 bp, Artificial
  Artificially synthesized primer sequence
tttagccctgccttcatacgct

SEQ ID NO: 30 - DNA, 20 bp, Artificial
  Artificially synthesized primer sequence
cgtcgttgccgaagaactgg

SEQ ID NO: 31 - DNA, 20 bp, Artificial
  Artificially synthesized primer sequence
tctagaatggccggaagtgg

SEQ ID NO: 32 - DNA, 20 bp, Artificial
  Artificially synthesized primer sequence
gagctcctagttgtagaccc

SEQ ID NO: 33 - DNA, 20 bp, Artificial
  Artificially synthesized primer sequence
tccactgacgtaagggatga

SEQ ID NO: 34 - DNA, 19 bp, Artificial
  Artificially synthesized primer sequence
atcagctcatcgagagcct

SEQ ID NO: 35 - DNA, 2049 bp, Oryza sativa
  promoter (1)..(2049): promoter of the gene 1
gcattcactctcccgttcttgatcgctttaatgtacattttggaatattgcttatcatattttggaatattaattattactttctccgttttaggttataaaactttctagcattgtccacattcatatatacgttaatgaatttagacactatgctaagttttataatctgtctctctggttctattgacaatcataataagagggatccagtcctctccaaggcgcatgataaatttcttattcctacaagcactatattaattaatatagtgcttgtaggaatgagaaatatagtgagctttaggcctgatgttttggggtttttttcttggtcgtaaataaaatcatgggtaactttggttaccgcgaaatacaggtgaaaattaaacgaaactctctggctgagctgtttgagagaagaaaaaaaaacttttccagcactatgaactgcagaagtaaagctttctttctcatcagaaagttattagcatgaacactgaataatacatggaaatttcagccagaaagttggaaaatgaaactaattaaacgccttttaactactaccggtggtatcgtgtacaatcggtaatgcattctcatttctccaatgacctatgatgaatgcgacaagacaggtcccataagatacataatcgaaatggttagtactattagtttgcttcatttgattaattctaggggctctaattggccggacaaaatttgagaatatattttcttttttaatattttgcatttatatatatacatgggaataatttgtaaaaatatactctcttccgtacaatggtaacgcggaatcgctctgcggcaccggcgcggaccttgccagtggtgcggcaacacttactaatcaccctggccttaatcaaagcaaagaatatacatagctaagcagctgtgcaagctagcttcctctgctaagcagccggtgcgcgccatggttccgcgggaccgcgttctccaccacgctcccgagtgacagggcaacgcgatcgaccctattccgcgcttccgacccgcggaaccgaaactatgtcgcgtgtttgtaacggtccagcgttcccatcacgcgggagaggtatatttttgcaaatcattcccatgcatatatataaacgcaaaatattaaataaaaaggtatattttaaatttttattcctaattggccgtcaatcccatcggtgcagtaatgtgaaaaacacaagattgttcctagtaatgatagcaagagtttagcataagctcaagtaacatttcaaccacatgaaagttgagacacctgtgagggcaagacacctgtgagggcaattgcgaaagctggctcgcatttccattattattatgatgctccctagaatttcaaatagtcatgtggttggtatctttactattctttatttatttatttattgccattgcacacatgctaatttaaaagaccttctaattacttgcctggttgtcgaaaaatatacatggtacaaattgtctcgccaactacttattttgcaattcctttttagccaaaaccactggcttttcaattctcgaaacataatagaactcgttcttctaaccattttataacggtttttaccgatctatgtgaagtttgatgttgcacaaatcgatcaacttgttatagttaggtgacttaaattacatagcatgatatatatttatagaggattaaggtagtggtggagcgcacttgtagtaggatgtgcattcctttgtctcgtcctttctcacacacttgctggttattaagtgggccatctgacagtgcatacaggaactttccccttctctggttccccctcctccctctacagtactctccccaaaatctctcctcaccggcccctcttccccttcgattccagtggcttgccgccagcgagggttggaggaggggtccggccacctcccctctactagtattaggttgttaatctgttattaggtcgtcaggtgtctcctgtagttagggtttgtccctagttggtcttttgccgg

SEQ ID NO: 36 - DNA, 2345 bp, Oryza sativa
  promoter (1)..(2345): promoter of the gene 2
attgccgatccatctacatgagtcaaaaaaaccgtacacaaataattattcttacaataatgacagtcatacaataattataagatatctttattcatacaaaaatcataaaatattacaccaaataattcttaaaaactatttgtaaagttttaaactaattttaatgtgttttacttcttttaattttcattttagtttgttttctattatttaagattaactttcactagagttttccttctcaactattttataaaaccttgtttagcaaatgaactaaatttctatatattctttcttccccacacacattttctctctcatcaacttacaactaaattttggagacaaaaaaagtgtgcaaaacataggtgcaaatgtagtatgacaaaaaaaacattggaggatgaagttgcaaaccttttaggcacctccgatttgtatgtaatcaccaaaataaattcaatagaaattttggcatgacctcccctcttttttgaagaaattttgaagcttgctcgggcagctggaggaggaaaaagacatatatataggggtgggactttagtcctggttggtagcaccaaccggggctaaagatcaccgggatctttagtcccggttggtaacaccaaccgggattaaagatcgtataccaaccgggactaaagatcccgggggtgcctgacaggccctgacagcattcgagccgggactaaagatgatctttagtcccggttggtaacacaaaccgggactaaagatcaaatatgcccgttacccttttgaaccggaactaaagatcatctttagccccggtttttattgcatccgggactattgtggaaatcggccgaccgacgaaagatggtttctccaccagtgaccctttatcaaactaccagccctccatagataataaagttcttggaaaagcactagcgacagtcgtagttacgttgacagaaactgtacggttaagaaatattcgtgagtgggtacggcaaggatggtccaaaggcacccgtcttcccgataaggtggcagaagaagtagttcactggcgcagattaacttgtgctgtgcttatttctcataaaaataatgggcaatggtggggcagcaaggaagcagatgaattgtactggagtttgggggttagaagaagagatgaagcatttgatcgtctgtgtataatgatttggcctagaggatcatgtacggaagaagtgttggatgatcagcacattgccaaaattgagctggatatggcaaggagagcaaggaggtggccgacaaggaagacggggatgggctcgtgatcgtgaagaactagtgtctagtgagatgttagctcataagatattaattacatcaatgtacgtgtagctcgatctatctgtctatttactatttcaacatgttttctcagcgtatcagttgtatttatgctccaaaatttactgtacctagtattttcattccgtgcaattaattagtagttgtatttgctgatgtccagaataaataaagtatatctactagcagtattctttttctcttttttttcccaattcatcttgtttgcattgctcaactgtatcctaacacgaaagagttgtaggactagtctgagctggtcgctagcccctgccacaagccaaaccaatcttctcatctccatcgacgaatctgatcagccgtgctggggtttgggtgtagccggcctgcggccggccgggactccgggagtttcgtcgatcgacgagctaaagccgccgccaccatccgcgcggtcaaaggctagggtgctgtctgctgtgctgtccagcgcggcgtacggtacctcgactcgactcccgactccggtggtagcgcccgacggcccgatgatcttgggcccagctgagcccctgatcactgccggccaccccgccgattcctactgggcacagttttcggccgagtacatgcaaccacaggttctacccaacccggctggaaccagtcatcagtccatcacgatatacattcaaaccggaggcgctttccccttctctcgttccgctcatctctctacagttcccttcccaaaatccctcccctcgggcccctcttccccttcgattctggcggtttgccgccggcgagggttggaggaggggtccggccacctcccctccactagtattgtatagttagatttgtttttaggtcgtcaggtgtttcctgtagttagggtttgtccttagttggtgtttttgccgacgagtctccatgggtgccgttgtggtcggcggcgacggcagttggtggcggtggaggtgcgagatccaaagacc

SEQ ID NO: 37 - DNA, 2897 bp, Oryza sativa
  promoter (1)..(2897): promoter of the gene 3
ttatgtcagcaatataagcatttctgaaatactgatctcaatttacggggattcaaattattttcagactatgaggatcctaccagggggatactaacaattccaaatttaaaatttgatcaatcactgaaaactaatattttctcttatcttcaaatttcgctaaacaacctaaagtgattatatttatgaacggtggtatgatatatattttttcaagggaactgcctgactacctgtgtctcgctccagccccatcagatggcgaccaagcgcccgcgtcgcccgccacctccctcctcttctccgcatcgccgctgcttgagcaggccaccgacaagccagctgaccgcaaggatcacaacggcggagctcccttccttcccccgaggacactacagcgggcaggtgcaccgaggcagcggagaaggtgccgtccagatccggggtgacagaccagcggcaaggtcggcgggcagcggggaagagggtggtgagtcagagtctttgtttttttctgaacaatcttactcagatatagaaacacgttattatttatttacgaacatatacccccggcactcgaaaaaattggaaataacaattaggaaatgtgaaggtccaaatggcggatcttaatagagtttagtggagctcaaaaattcaccgttagcctccacatcttcaaaatcaaaatatattaattagaaagaaatactcacgaatgtgtatagaataatagaaagatgtactagaagataggcgcgcttgttggcgtgatcgctaattaagcatgtgtcttatcttatttgttggagattatatatatgctatttaaatcttttttggaatttacatttttatagcagttagttacatactcataaaatttcttataaacaccatacttttaaaggtatgttttagtgtgttcttagtttcatgtttgtagaattatgaagagaaaaaaatgaaagtatgatagtaatatatgcttaaaaagcattcaaggtggtagttagtttaatgtagaaaactgtatttttggaatcttttgccacttttgtgaattgaaagtatttacatataatctgtttgaatttggggaattataagaaaatacatatactagtactgattgttttgcattctctggttcttttttttttcattctctacgaatatagatataggataacatcattgttaacttgcttgttaggaatttcagtttcacccattgtatttcttggagttgtttatatccacacttccctaaacagtaaaactcgtagagttagcattaaatgtttttacaatttacaatttatattttatttgttatttgaaggaaggataaaagctttgctactttatggtttgtatgttaatattactttgataatacaggatgctaagggcaacaacaatgtatatggcaaaagtagtctatagagatgagacccatatatatgaactttaactattgtaaccttcacaaactatatggatagcaaatagtattagtaggagagaagagatagagacaaataatatattttattctctatgggcaacccatatgcttatgggtagcttttgttatttttttttatggactaattccacaatgttgctaggtagctgaataaatattatattgcttatggactacttaattttgaagtacattgtggatgccctaacagcttggtttggcacctacatctttaatcttcaattttcaaaataccacacttctttattattattttaggatttgatactgcaacatgtttatactgtccacaaggcttggcgttcagcccgtagcaatgtttgtgcaccaacatggctaagccacatcccacgtgcgtgggaggctgggcagctcagcacgactgcacgaggttggctcagcccgcactgtccacgcatagtgatgggccaacatggctcctggctacgctcatgggctgatcaagcccatccacgagagagattgacaaggacgtgactctccgcgcgagaccaaacacccgaaattaccgctgtggagttttttaccattcgtaatttctcgtatttatctcaacttttaacggtgccacgtggtccaatctgcagccggcaaaaccgcatgcttgagaggatggatggatggatgtgtatggtggtgtctgcacgtaagcatatacgtagtactacgtacaacattgtctttgggaaaaaataacctcatatataggccgccgaacgaggcgcagaaaaatgggaaactgtttaacagctcgagtggacgttcacccgtttattgtatgttatctaaatagttataaaaaaataaaaaaaatatgaataagatagatcaatatgtaatatatcaatacataaacatagaagttaaaatttaacttctacaagttgtaacaaacaaaactcaaattactatatgtatatttacaattaaatttgttatttttgttacaacctgtagaagttgaatttgaacttgcatgtttgtggagtgatatattacatattgatatatcttgtcaatttttttaaaaatttttcataaccatctagttcatatgcaataaacgggtggacatccactcgagttattagaatcctctcccgcagaaaaatggtggtatgacacacaatggagccgggtggactgtggaatattatttgctagtcggctgtgcatcggagcaattctagccgttagcggtgcagcggaagcttccggtgcaacccatttccacacggatgccaagttgccttttgattcaccactggctgagctcactctgacccgcaaacgccagtgcccgcagcctacatatagccgcatctgcccatcaaggttgagcacagtcatcgagcct

SEQ ID NO: 38 - DNA, 2370 bp, Oryza sativa
  promoter (1)..(2370): promoter of the gene 4
aatgagtagcacgaggactcacccctggtggttggcctggacctggagagggcgccggagacgggacgcatgggagggtccctatccctcctgacggcggaagcggaggagaaggagaaggagggcggcagaggcgagagggccgcggaggcgaggagcgggaggcggcgcgcgaagccggaaaacgagggcgtgggggtgggagcgacggcggcggaagcgtacgggagctgcgccgccctggctcccctcagaccaagcatggccgcgcgagggcgaggctccgatggcggtgggcgctgctgcaggagaggagaggagaggagggaggaggaagaaggattggaagacgagtgaagccaagagaaagatcccctcacctccgcatcattcgttcgttcgtcaacaaggccggagtcgctgtatgggctcacttcaacttgggcctgacatgtcatccgacggcccattttacttactattgggccttcctgtcctccttccacctgcttccctaccaagaatgaggcccgcttagaatgcggccaccatttccgttactcgaacaaatcaccgaacttctccctctaaaaaaattcagcaaaccagcatcaccgaacatatggtgatccggaccctacctttaatcgtgcatcggtctcttatcactggtaatactagtattcgtacagtactggtaatatatattgcgaggaattaattgaaagcccatatatgaacaatgagattctctctcatcgttctgccttttactggcggtagctacgtgaaagggatagacattgactgtagtactagtatctatatgtatcttgactgcaaacagcagctattagtacgtataccggtctagaatgtaccataaaagtactcaagctatatggtcaataatcactcaattggactagtatctgatcctggatatgcgtttgcttaattagtcaattatgaatcgacaatgcatgcatctggattcctgattgcttttgtcctcgatcagcaatcgatcgattgatctagtctaggcatgcattaggcacataagtatgatctgcatctgcatgctagctacctgggaattttatcgatcaccaaaaactatatatcctgatcagtcgcacgcgcgtgcgcacggatgttgaaatgtacgagcgccttgggtccgtgtgaatgagacaatttatcgatcgatctcaaggaatataattaatcgatcggcaggtcaaactcaaaacgaatggatgattttctagctctgaatttctcaagtctttaaaaaatagtagcaggctagcacacccaccttcttcaattcgtcgtcgagcggttgcgatgccatcgtcgatgacggtcatcactgacgatcaatgctgcgctggatgattttgctttcaaagaatccaaatataatatatatactcgaaaacgacttcattttttgactgaagagaataacttgtgtttacctgctagccaattggctagagactgactggagagagcgaatagccgtgagacagctagctagctagcaatctggttttaccgttcatctttttacctagataacttcttgtttgtagatagatgaaacacatcctatgaatctggactttgaatgggttatctcagttaattccaacctatcctttgggtaagagtacttgactgggtgacctccctctaactcgaaccaacaagtctcttgtcttgtcttccttccttccatgcgtgaatttttctatggttaaaattaataactacagtagctagctggtacaaatctccaactaaattttataggcaggattttttcaaggtagcatagcatactagctcagattctgaacttaactacaaatgtgtgataaggataaaagatttatacatactgcatgttggaatttgaagtccattaatgtagatattggcttaactttggatgaaagtttacacataggattgaataagatttctccttttgttgttcgaacttactctctcatcatctttggtcattacgttcaaaattttgtaataagtggtttctatcttaattgaagaaaatgccagttcttttttttttaaaaaaaaggccagaatattcatcatataaaaaaaaacttaactggcacaataatatctactcctgtctctcaccccagccgcccaatgaaaatgctctgatccaattctcccaaaatttccactgtttcgtctgttactgcagaaaattaccactgtttcgtctgttactgcaggatatcgttccgaaacagggctactgtttctataaaggcacgcacattgcacgtacggctgcacaccacgctcacaaaaagctctaga

SEQ ID NO: 39 - DNA, 2284 bp, Oryza sativa
  promoter (1)..(2284): promoter of the gene 5
ccaatatccacaagaaacagaggacaatctcgtccatctcctgaaggcgcagcattacaagacgttcatactgttgccatacaacacagagttagtttaattttactgtcttcctacataccaaatttcattcccgtacgaacttgctaagtgtttcatatgtaatgcatcccacgcacattgcagattccactgggtgcttttactcatcgacctggaggcctgcaccgtcaacgtatatgactcaatggataaaaaagagtctacgtttgacaaggttttcgaacttatagacaggtaccgtcataagttcctttgttaattaagaaaatcttgttatgttaattgctactacgaatcatagctttaaactccatgtagggcttggtatcggttccgtcatttggttcgcggcaaatggagagaaagacttaggcggaagttcaaatttcctgtgagtacacatgctctacatttatatttctccgattcaaatacatacaagtgtatattaattagatctctcgttgtttgtcatttatttgtagtgcgcaaagcaaaagcagggaactaacttgtgcggctattacgtgtgcgagtattgccactgccttgcagaccaaatcatcaccacaagagagctcgatgtacgtacaaataaattcaaaatttcattacgtagcgatttcttgtttaattactaatcaatttcatacattcatatagtttattcgcatgagggataacctgaccacacacaaggaatttatcgcggcggttcaagaacaactcatgggattcatcaacgaagaaatccttgatcccaagggtgaattctactacgacggaaacacaattcaccggtccttagcttctgagctagcagcgagtactactacgtcgaaatcgtagctagctaggacatataatggattgtaattaatacatgactacatatgtttctatatgcatgtgtacacattttctataatgtaaatatattttgttcatatatatatctatatacatatgcatttgcataacatatatatgtataaatacatatatattatgcatgtatatacatataatataatataatatatatatatatacatatatatatatatgtatgcatatatatgtattgaaacatatatatgcatggtttatatatatatatatatatatatatatatatatatatatatatatatatatatatataaaccatgcagcaacagggcatgcaaaaaaaaaaaggtcagctcaacaggggtaacaccaaccgggactaaagatcgatctttactcccggttatttcacccgggactaaagatagcgatctttagtcccggattggtactcccggtttggaaaccgggactaaagggggttacgaaccgggactacaaagggtttctccaccagtgaaggtacgtactatatatatgcagtatgctatcacacgtaccaccacaagaaaatgtgccctcaaatatgtacacaaaaaactgtgaatttattttagatgtacaacacaatgagacacatgtgttaacacaaaagacatcaactaaaaacggtatatgcatatatggagagaaaaaaaaagacatattcaatgtcaacacagttcacactagctactacgtactagcataatgctttgtttttttccattactccattattttgtaaaacaaatattgacctgacgttttctttggtggtacacatatattacgtgttgcacatttaaaaacattttatatatgatgtttaattgtcgtccaattgggagagtactgagtacgtgtacttcggtgccataccagagagatcattctgtcgatacggcgtgtgcaactaaaaatttgcatctgtctcatggtgtttatatttttactaagtaagatatagattttacgtgttttgcgtgaaaattttatatacaaaaatcgttttaaaatatcaaataaattaatttttcaaatttataataactataactcaatttatcatgtgctaatggtttttttgttatgcattcctttaacttcatcttcattagaaaaaaaaaccacctgaagctcttcgcacgaatgtcgatacattcccaagcgtgtataaattcaacccttccatgcgtatttgtgttcaagaacggagagacaaaaccagctgcattatagcacacaccagtcgtgtctacgtacacaaattgttgtcgcacacataattatcatccatcgatccatccatacaaacgtacgtagaatc

SEQ ID NO: 40 - DNA, 2414 bp, Oryza sativa
  promoter (1)..(2414): promoter of the gene 6
caaatttcatgtggatggtcctgatcacatcctaatggcggttccggggtagtttggtcatttcgcataggctgtaaccgtcatacctgcctataaaaggaggagctcactctcattttcaatacacaactttgagctgaattacaagagactgtattgtatcttttgtacattagaataaggtagagagctctggaggagtgcccagaagtttggcgctcatccgacttctacctcgacgagtgtagcttcctaggaggaattctagaggagtactggagctgtcggtacaatctgctccgggttcggagaaacttcttctattaagtaagcattcgtgtttctttcttatgttatttaattactttgtttaagtaagatatgtttccatctttgcggattaattgtttagtatttatactaagtactctagtactaggactccggatagagaaggaagttcttgcgcaagtattagagtagtaattaataacttagacgtggtgtctaagttagagattacctttgtttgttgtgtatgccacggatttgtcagaggtaggcggcatgtggtgacagccctgaatcccgtcctagtaatcctccacgttcggatacatcaaagagctatagcaggaaccacgctggtagaccagcgggggtcggttcccgaagcaacagatagggaattacctttgcttagcttagataattaaaccacatagaaagtcctctctagcctagtctacctatcatcagttgttgtccttggatcgtcttacgcttaggtagattacatacgttccctgagtttgatatccttttggggtcaaccgaagatgaagtgctacagcggtattccgtgcgcttgcggatttctctgtggtcgtaagaaataccaacaccacccgccgccggtcatcccctccgccttcgtcttagccttcgactcagccgccgtgctagtgagatggtgagcggagggatttgagagagagggggggttgagatagaggagagagatagagaggggtggggaactgacagtgggtcccactaatttttttaataaaattactgactggactgccacgcatatgccacgtaggaccaaaaccaccgcggattgagccgggggggttatttgtcctgtttcaatagttggggggtgtagaatgtccggttttcgagttcgggggtgtaatttggtcgacctcgatagttcaggggggtaattcgtactttttctgaaactttatatgtgaattgtagatagtgtccatattcaattttgtagtgtaacgttttttcattccagattgtttatgtggctataaattcgttacaaactttcactattttgataaaatgaatttttgaatttttaaaatgagctcaaatggagacatgctctgtagagctcgacgctgcaagtttatagttgataactttttaatttgatggcccttggagtaaaaaatatatgttacaagttttatgaaggatgaagtcaaatgaatagcatctatgggatagtcaccaaagatcctgggttggaattctatgccatagtaacgaaagctatatgccatagtcccgtgttggaattctatgccatagtaaggaaagctatgccatatatacagtccatgggttggaatattctatgccatgcatagtcgtccccggtcatagtcctttcgttagtgactatggcatagcaaggaaactatggcactgagtgagaggccctgggttcgaattctatataccgcccgtgcgcatatttcacatctaaaattatgtacttgtgattaaaaaaaataaaaaactggaaatcccgtgtaatagaaaatttttcacacgcccaaggttgcgggtgggatcgtaaaatacctatgtaaatgggttatggaccatccgcaaaaatattaatttgctgtagtagcgtcggagatatatttaattcactacaaaatacaaaattgttaaaatttttagtgtgcaatgggagtgttgtaattggatgttaaaataactagctgaatatataccccaccgcgttgctgcgggaaattaaattatgtaatgtgtacaaataagatgcaatataattattaaaacccaaacaaattaatacttataaattttgagccaaaaataattcagattgcatggtgttatgagagaagaaagaatagacaatttgaaccatagatctattatctaaaagctataaataattaggatgatatggtttgatgagagaagagagaaataacattaaccgacacttggtggacgaaggatctcggagcccatgcatcgctatatatagtacacctgctgtagtacttggcaactggagactggagtagtagctattgctagtgcgtacagcacatcgtacggac

SEQ ID NO: 41 - DNA, 2957 bp, Oryza sativa
  promoter (1)..(2957): promoter of the gene 7
acagtatacactgacttaggtggtgtttagatccagggacttaactttagtccttgtctttagacactaatttagagtattaaatatagactacttacaaaactaattacataaataaaagctaattcacgagacaaattttttaagcctaattaatctataattagagaatgtttactgtagcatcacataggctaatcatggattaattaggctcattagattcgtctcgtaaattagtccaagattatggatgggttttattaatagtctacgtttaatatttataattaattttcaaatatccgatgtgatagggacttaaaagttttagtcccatctaaacagggccttacggcatagagccgaccctcgttagcgcagtgcctaacttcaatggtgcaaagaccaagtagccgagcaggttttcaatttcgaaattggtttttttctggggtggaccaaaatttcagaaattttttggctgaaattatttaaatgttttactgattttgaatgaatttaaataaaatttgaccaaattcataaaaatttaaaaaacccgaaattttcggatgagatatgagcttgccggtcatggacgaaattaccgaaatttcggaaatctcgaaacaaaatttcaaaaccctggagccgaggcggttggtccatggtggccatccccacgctgcggcggagtccagatggcgcctgccgcttcccgagagggggaatggaggaggtggtggctagggcgaaacgactagcacgcccccacctatgtctcgctgttccgggccgtcgcgctccacgcccggaacggccattgctattccctatcgcttccgcgtctattgattgcgtcgccgacgcgcggcgggtcatcctgttcgacgagttcagcttcaggcctggcggcgtctcggccgtgtccgtccacgggtgtcatggcggatggccgaggcccgagaggccgaagctccaggccgccgaccctggcctaatggggttcatcctcatctcaaactccctcttctttcagatcaacaaacgagtccgactacgctgaggccaccggcggcgccttctgcccgctcaccatcgacgatcggcatggcgctgccggaggccaacattgcgcagttcagctcatgcttcgccggtggactgaagcagctgtgacgaacagcttgttgcatttcctgcttcttactgctacgaattgttgcagagttcagaatttgttcagtttaggtgcttctgtaatgctagcggttggattgcaatcgttgatcaatcacataagcgattgcaaatttgcaattgtcaatcaatgaaaaagtcagataagctgtaaacttttctacatttttttcagagatggggcagaataaggcctgcttcgatcagcttctgttataaaaagaaaacactgaatattcggttgtatactgcatttcttcttagtaaatgcacttttctaaagaatcttgctcgcgcctcttgcagaagacggaaggggagaggaagagaaagaaagagagaaaggatagagggggagaaaaagaaagacagagaggatgatatgtaggccccatattttttttttgcgaatgataaatgggtcctatatatatgtttttaattctactaccacctaaacgtcacagacaagtcaatactgtcatgtagatgtcacgtcagcgaaaccgtcctctggagggttgagatatccagtattgcggtttaaggattcgaaatagatttggtcacaagttaagggagtcattgtaaacatatttcaaagaataaagacaccataggtcccgggcccaacagaggcccaggacgcgagggttttatcactctccgttgccggagtccggtgagaaaaggaaagaactcgccgtcgccgaccgcgggcgaggtggccggcgtcccctcgagcggcgctctcctccacctccgcggcacatccatctttttccacgaccgcctccgccgcgccctcctattctccccggcgatggcgccgcctcttcttggttcttcagccaccctcaaccccggccactggttcctcggaggtatatacgcatatatgtctcgtctctgctcctctaccctgatctttcagttcttgtttaggaactcgagaatcgagatgaccgaggaagaagccagtgcattctgaaacaaaaaaaaacccctctaatctttggttcagttgcatatggtggctttacttgggcgcttcggttccaaatatgttagtccagtagattcttgttgcaatttattgtatgcttctgttatatctagtttacttgtttctaaaaccttggattactcaattccctaatcaattagtacagtttattttcccaaatataccagtttcaggttttagtttgtataaaatgtaggtgcattttattaattgatttgtggaatacttacctgttcttttgaacaagcaaatccaagattttaattcgtttatagctgcaaatcctttcgttttcaattggtactgtaaatcatacatagtttgttcgactaattcggttctaggtttgaaaagttgctctatgttaactgattttcagagtttctaccttaattaatcatttcgtggatgaatagataggttacttctttaaatcgaatcgtgaagcacaaacacgtacagcaaaatgcaaaaagttttaaatttcatcagtataaatatggcacattaattttccatcctgtattcatgtgattcattacgatttcatcaattttaatccagggctctggacacctgctaccctttgaaattacataactagcattgctgcatttctcaatggatcatctctcggaggaactgatcataaatattctcaagaggattgcaaggacaaacaatctgaattctctttcccttgtgtcgaagcagctctacaacattg

SEQ ID NO: 42 - DNA, 2301 bp, Oryza sativa
  promoter (1)..(2301): promoter of the gene 8
gtcaccactaggtagatcgatcatccctataatcatcattgtacgacgttgcgtgttcgacggctggattaattggtttgaataatttttggtagttaattatctgactacattttcccaattcttaccaataaagtcgagtttgcacaagctacatgtgtgaattcttttttttatatcttagatacacttttttgtcagagtttacatgagttttcaccaaaattgagtttccaccttccaccgcatatttagggtgtattcagtagtaggagtttggaactctccccgaagcaccgcatatttagggtgtattcaatagtaggagtttggaactctccccgaagcacacaaaatggagtgttgtatcagcgcatctatatactaatataaaaaggagtttgcccctcccaatcctattggtgaaatcattccatctatctcatccgttcatcctcatcccatctaatctcatccgtccgctgtacaccgtagctagtatatctcctctagcgattttgcccgatctcatcccaccagcaatttcggtccccatgcggtggctgcgcggcgcaaatccccgcgcggtgactccgcctactcactctgcgccttgctccccgcgcgcatcctcgctctacgcctcctctcctcactatggtccagcgacagtttaccgccggaccaacaacctcacgtcctcgttccgcgcaacgccggacctccttcatccttgcaacggaccaacgacaacgcaccatcgcaccgccagaccagagccgaggcactaccgcaccgccagaccggcaccgaggcaccatcgcgcagctgcccaccgctagaccagcgcacgccatctcatccccttcagccggggactgccccagacggtggggtagtggttccccctaccgccggagccgttgcggcgcacgccacgccggcgccgagtttgttcgacattctgtcaaactttcttcgagatgcttaggaactggtgatttggggactacttcaaggtggaggccattgggttagcctgggatcatctcactcaggttttcttttttttggatccttttagattccctttttattctcatgcaattgtggccgtgctgtgtgaatctaatgtctcttctaattgggactggaatcatgagttggttgggttggcattaaactgttttgagctagcgaagtcttgataaataatacttcctccgtttcacgatataagtcattctattatttcacatatttatatttatattaatgaatctagacatatatatctatctagattcattaacatcaatgtaaacgtgagaaatgctagaatgacttacattgtgaaacggagggagtagaagagttgttacttgtaagagatgtcttattttctgatcttgtcgagtacaaataatagataagagacaaacaatagatgagttgttgtgctcgacaatttgggaagatgtctgagtacaacacacctagctttatcccctataaatacgtttataccccccagcctgaaaagttaaccatgcaggcatcaaatcacacagcactaactcatcaagagctctacatatacatactaattaaacctaaatacgtagcctgatcagtttaggtttgtcaaacgccatggatatggagatgggtaaattgcttcatcgcccatggaaatggagcttaaactcgccgctactgctgctgctcatcgtccccgtgatgatccacgtacagctgaagctacgtcgtcgtcgcaagaacgccgccgccggcacgaggctgccgccggggccatggcggctccccgtcatcggcagcctgcaccacctcgccatgaacccgaaggcggtgcaccgcgcgctggccgacctcgcgcggcggtgcggcggcggcggcggcgtcatgtacctccgcctcggcgagctccccgtcgtggtggcctcgtcgcgggacgccgcccgggaggtcctgaggacgcacgacgccgcgttcgccacccgcgccatgagcgtcacggtgagggactccatcggcgacacggtggggatactcttctcgccgtacggcgagcggtggcgccgcctccgcgggatctgcagcctcgagctcctcaacgcgaggcgggtgcgctcgttccgccccatccgggaggagcaggtcgcccgcctcgttggcgccatcgccgccgccgccgccgctcccggcggcgatcagccgccgccggtgaacgtgagctggcagatcgccggggcgcttaccgacctgacgctgcgcgccatc

SEQ ID NO: 43 - DNA, 2512 bp, Oryza sativa
  promoter (1)..(2512): promoter of the gene 9
tatagcttgtgttgcgcacctcgaaagacagtggatggctgtatttataggctcgtatgccctttcatgaaacaaatgatacagcatatttttcttttattccgaatattgatacaaattttttaccaactgactattcagagtcaggacagacagttagtttgattgggactacgtagcaaaaaccttggaacctagcttactagtaaaattgcagtctacatacgtaagcaaatgggtctcgtacgttgcaattagtatgactgtttcttttccatcggtatcacttcgctaattaatcgctatcagatacgcagggttaatttaattagttgtgtttttatggattaggttatatagtataagatagttgttgttttttttccttgatgcatccatataatatgatgattaattggtaaaggagtgagtattagatactaaggggtacatgcgcgatgcccttagaatttttttctccgtcggccggaatcccactccaataagacaatcactaagtcggatcggcccacctgtgatagcccaaagtgcttgttatggatgacttcaccagtggcaggtccaacagaatgtcgtaataacaggtgaggtcatcgtccgagactcgacctctgtcggggtgcaaatggcggcccgtcatatataggtggccacctacctgtgacgggtggaattatagcaaccattagacatatccgagacccaataaataccctcaacaaccaactgttttccatcccaaccctgtacactgttcacatttcggttggaagggtaggggcatttttttttttgaaattggagttaaagaaaaactttgataattgtgtgatgaagttttgtgtatttgtgtatcttctatcgcagatggaacgaaattggatgtacggtaattcttgtaggaaccaagagtatagggatggggttgttgaattcatgaatatagcggacaatgataaaagaactaggatgagcgaatatatgctatgtccatatgcaaactgcaaaaacgagaagatgttctctcatagttcaggggtgcaatctcacctgatccatagaggatttgtggagcattatagttgttggaccaggcatggagagcaagagacaccggatgttgtgattgatgaagtaccagatataagcaacgagccaaattgagatggtatgtttgtcccttctccattaggtgctgataccatagatttcgaccctatcacaaatgttacatgatattgaagagccaaccataatgagagggattttaagaagtttagcaagttgtggtatattttgagatgcctttgtatctcgagtgcaagccgaagcacaccaagttatcatctatcttagaactcatgaaacttagggtaagtaatagtcggtcagacaagagtcgcaaaacttttcgagtttctgaatgacttgttgccagaggggaacgaaataaaagacaacatatgaaacaaatcaagtattatgcccattggatctggaagtcagaagaattcatgcttatcaaaacaactacatattatgttacaaaaagcacaccaacttggatgctttccctgtttgtggagcttctagatacatgcgagacattagtgaagatgaaggtcgtaagtcaaagaggggtggcctcacaaatgtgatgtggtatgttcctatgctaagcgcatgaagagaatgtttatgaatcctaagcaagcttagtacattgggatgttgaagaatttaaggtcgacggtaaggtaaggcacccaacaaatttagtatagtggtaaggtaagacacaataaatagagaatttacaaaggacccgagaaagctatgtgtatggatgtcattaatccatttgatgatttgagtagttgtcatagcacagggcaaatgcttcttgggaactacaagcttcctccctggtagtgcttcaagaggaagtatattatgcttgtcatgctcgttcaaggatcgaggtagcctggtaacaatatcgatatattcattgaactgataatcaatgagaaaggactatatttatacgatatctatgagaagaggttcagcatgcacgcgtagaccatgaggaaggactatatttgtaactttggtccagaatgattatatgtgtaattttaccgcaaacctgtttttgtctaatatatatatatgtgttatatttcaattaaaatgaaattgcagtgtgcgggtaaacctgccagatattttgaaaggaaagagtattgccggggttgcagcgacagcgatggttgcttggcgagttgccatgctgctgctacttgtgtacttctaccttacagaagatcttcattaattctataaagcagacactataaatagacaaaatacttggctatacctctacctctcccacgagctagtattggcaagaaacctagctcatcagtcatcacgtaaataaattaaaaccaacaagtcatacgatctccatccatctcacacg

SEQ ID NO: 44 - DNA, 2272 bp, Oryza sativa
  promoter (1)..(2272): promoter of the gene 10
tcattcaccatgtgctatggagacaacagtggaagtgattttctggcctgaacccagctctattaatggggaaggcttgggccggagagagaatacttggggatttggttatacaaatggacactaatctgtacaaggatgtcctcttacattattgcactcctaaaatgcaacctaaaaagttatagaaactccattaatacattgagaaacatagagttaattgcattccgaagcaaggagttaggatcgacagagcacgatttcggttttgaattgtgatagtagagagattaatttggttgttggattcttcaattggaaattcactagtttttcttatctaacatagaaaattagttatttctttggtttattgtttgttggtgcatgatattatatactaatttttatttcatccacagttatacactgttcacactattatatataaactgttacgaaaaaaaaatctgaaaacttataatttagtgtatataacgcgtatatctttgtggacacactcataagattgtttaacagcggaatcatatccaaaaattgaataaatcactgggattatttttattttatgatagcatggaacatgcagacacacaccactatgcatgcactcacaattgtatttccctaaaatacactcaaaagaaactgacacacgccatttaggataggagaggtattaatcctaggtatcacaatggtcaatataggtgattaagttataactaactacctgaattgattgatgttgcaaacgattgcgttagaaaagagagctcttacgtggatactggattatacagacctattacatttacttcaacaacttgtgttggagatatgtcttatgtcgagagaaatcactcgctcaccctcacccactgaattggtagtaacacaaacacaaattctgaactttttcggatcagctatagtagactgaagaacagatttcggatctgctacagtatactgaagaacagagtttccagagcatagcagagaacttgttcgtttgattgcagcttttctgttttcttccttctatttattctcctccacttagggagtttttttaggaaactaagggagttcgttacattcacaattgaccttaataatgtctggatcgatgaaatcgctacatggtaactgaacaactgaaatgaaggaagctaagtctaacaactgattataagccacaatacttattagtgataacaaataatttgtgggtaaaaatttcatatatgtgttcttagcgacttaaaaaccaatgctggaaaataaactatgatgaaaaaacatcaaaatcaactctaagattaagttttaaaattcaaatttagctatggctgataagttgaagaccaaatgatggaggtttcaaattatactgtacttcagccatcctgtcacatttaagtcataaatttacaaggctctcgattcattcaatagctataccttactctctctttttgcagtaagataattatacctcatattccttcgttctcaaataactgtcattgttgacactatgactttcttactttgaccgcaattttgtacattttaatgattatattatgatgatgaaaattgtattattatatttatgacactacaatacttcgataatataatatgttttcaagtatttatatttttctgtaaaaaaaagacaaacaatcaaagtttaaacttgaagagtgacactgccaattatttaagaacggagggagtacaaaactatagttagaagagctccgttcaaattttccgattccaatatatggacaaacaaactggtagaaaacacaaaatacacatgctgactagtactttgtacattctgatcttgtacactgacagaggagaggtcgcaggagtacagaataatccatcttcacgtgtgttttgtgtagttttctaccagtttgttgaatggcaggttctgcagctaaggatgttttacttggtcagaataatagatctgctccaggactcatgtggctataagaacagtaaaatatctcaattcagagagtaaatataaggtataattataccttgagaagaacagtaaaatatctcaattcagagaaacttcaccaaacctagaaacttcaaagtctctggtggaaaagaattccaggttttgcgttgcaattgtactatatatataagagcctttgcctctgtaccatttcattgtgccaagaactactcaggtcttgcagca

SEQ ID NO: 45 - DNA, 2850 bp, Oryza sativa
  promoter (1)..(2850): promoter of the gene 11
aggattttgtgatgggcttggcccaactataacctgggctagctaagaatttccaaatgggcctgatccaatcacctcatgagtagtccagtagatatctcgatatatctctactattataaaaattgaagatgtttttgccgatactttggtacgtcatccatgtatgagtcggtttttaagttcgtttgcttttggaaatacatattcgtatttgagtcaatttttaatttttaagttcgttcgcttttggaaatatagaaggagtcgtataagaatttttttaaaaaaactcgcatgctaacttgagacgatcggattcataattgcagctcataattttctaaaaacagaatatatatatccaagcaaattctcacataactaaaccatataacaataatagattaaaataaataaataatgttcacccggtgcaacgcatgggcatttgttctatatattatagaaaaaaaaagtgaaaattgatggcgacaaaaagtaacctatataaatattaaataagttcaatggaacgctactgcatgcgtgtttgcatgtaaacgtgacgaatatagtttatagcattatagtctaccttggttagtttctagaataattaattaatcatgtatactttgtttcatatctggcaattgatatctcgatatagcgtgccgaagaatcgcgagaggaatccccttttccttctgctgagaaatatgaatggttgattgctctactacttaataataaaatcacaatcactctctagtctctactttgcatctttacctaacactccacatgtactagtacacacgccatgataaaattagtgccagtttccttcaataaataaataaattagtgtcaggtagggtccattgagttgatcatcttctttttttttttttagaactgagttgatcatcttatttatacttgggtttctttcgacaggccagcaatgagtaatataatctcattacatcacatgcatggtgcgactatagtactttaattacatgatactattgtgtactattgctgcacacatatggtggataatattgcatattatattttaactagcaaataattaattagcataggctgaccaggtgtccacttgcaatttttttaggggagaaagttgcagcttaattaagttcagatcatgcatgatatagctagctattgcgaaggacgtgcatgcatgttccaatcgttagctaagcacgtaccccccaactgtcattaaccgttaattacaaccgccacattaaacttaaaattctgtaatgttaaattgtttatttattaaattaagataatcacttcgatccactcgatattgttgcatgtttttggataacataagtatcatgggcttaatttgcaaaatcttaattaattttttacgcgtcgccctatagtgtgtatatatatatagatactcggttagtacgcaacttcggctggagtataatttatcatcagatattcagatttctggaagcttaatatttaatttagttagtcgtaggtatatatttcacgtggcatcacactgaccgagggggggaacttggatgaattcgattatataaatatttatacagtatactgctcttgcaacgtggagtaaggacacatatatagctccaacttctaagtttggtgttgggtctggagtggagttgagctgtgagctgtctaaacccagctatacttctctagttccttttgtgatagggctccacctagctctgctactattttaggtggagctgaaactgtttggctgagaggtgaagctggagctgtgccaaacatgccctatatatactccttatgttctgaacaatcactacctccgctttaaaatagttatctttctaaaatttaaaatttatcctgaaagacttatcactctagttactacttatcacattcatctcttttcattcaaatttctttctattattgtatttttcaagtactactcaactttcaaccgtatatcatttaggccctgtttagatcccactctaaaatttttcatcctgccacatcgagtatttgaacaccagcatgaagtattaaatataggctaaaaataactaattacacagattgtgactaatttgcaagacgaatcttttaaacctaattgcttcatgatttaacaatgtggtgctacagtaaacatttgctaatgactgattaattaggcttaataaatttgtctcgcggtttaccgacggattatgtaattagttcttttattagtgtccgaataccccatatgacaccctatataatacctgacgtatcacgacgaaactttacacccctggatctaaacaccccattaggagcactataatctcttttttaaccattatgctagataacgtgttttttggatggagaaagtactccattcatcataaaatataataaccgagaatcaaataaaaagtatctatgtccactcgtagtattttctaggttgctatattatgtaaacagagggagagggagtaagtgtgaattactaccgtgtactccgatctctctctggatatacatacctccccgggagaaggcgaccgatactttccccttctctcgttcccctcctccctctacagtactctccccaaaatctctcctcaccggcccctcttccccttcgattctggcggcttgccaccggcgagggttggaggagaggtccggccacctcccctctactagtattaggttgttagatctattattaggtcgtcaggtgtctcctgtagttagggtttgtccctaattggtcttttgccgacgagc

SEQ ID NO: 46 - DNA, 4429 bp, Oryza sativa
  promoter (1)..(4429): promoter of the gene 13
actctgaataacactgaaacagtccattgccctcaagtcccatatgcctatggaatgtgtttaaccttccaggcttccagctcagaatgtgctcagcatgacagactgaaggacaccgttattaaccgccatttgtcattttggagcgtgctgtactctgtctggctgccatttgcgcctataatttcagaattcaaatgatactcctgcaagaaatgctactcgactggaataccttccatcatctgttcatcttttacaggaagaagatacctgtccctgtactatttcgaatccgagtctaaatttgagtagtcaattgagggtttagatcatgttttgcgataaacttgtacaatattgaaatactcgacagttgatagcactttgctttccaaaatttgttccgtagtctatatcagtattttggtccctcattatcttggtcgagagagggtcactaacaaaataaccaaaatgtaccttttctaatgcgattgtttttaactgcacatccatcttcttttccgcttctttaccgaattattgtcatgaattgtttactccgtgcttcaaaaaacaaattttcttttttttttaaaaaaagaaactttcttctactgcgttgcttggactacttgttttaaagttattagataagtttatttctcaaaatatgtaatttattcttacaactaaacagattactgtcacgataattcgatgaataatctgtttcaaatgctaaagcagatcagcggcacctactgaataatcccgtgtccatccctacttttttttagacacaatttctccatctccctcgaggggagcaagccagcaagatctgaaaaagggttttaataaaagcataaacaattacttcaagttcatgcgtctccatttgctatttgagcaaccattttcaggagggaatccaatagttggcacggtgaagtcagagtctagctttaccaaattgagatatcgcactctcgagcgagtccctccattttagaccacacctaataacatcaagatcatgaatcctcccagatgtgaacctggaatactggaaacacgtgtatttcgtgcacaatgagcaaagttagttttacatccagaaacctgtttcgcggcgatcatttcacaatgatgtttcgctgagattttttttatataatggataaaaatccggtctctatatccacaagtgtatatacaaccaaacatttcagaaaaatattattttactataatattctatagcaatagtcaagattcctttggaatataggaattttacaggacttgaggtatgttccttttctcttacagataataaattatcacattcatgttttcaaagcacctaaatggtacgtttttttttggtaaaaagcttcctatttctatatataagtttcttgtaatacatctttagatcaacttttaaattttgtataactaatttattcacatgaaacacaagaattttacaagggccttcttcttttcctcttatagattacaggttatcacagtcatgctttctaagctgtgatttttgcaaacggtttctacatatataagttccttagaatactttttaaaatatttttttaactttataatagttacttcattcgcttgaaccctagaatattattttagaaagaaaatcaaataatccatcatctatttatccatttagaacaacagaactaggcctaattagttcagttcctataaaaatactctgaaatttctacattcctctgagtgatgaaacaggtggcagcagcagattggtcctccaaaatttcgacatgtgaaaaaggaaccttgctactacacccttcaccagtgagttccaaggcaaggcatcactgatcagtagttgtgctgttcccatctagtcccctgcttcttgccatgaactcaagtgaagggcacctcctttttcccctacacacaaaataaataaataaatcagtgctactaaatcacaagctggaactttttttccttttttttttcgcgtcggtgaccgtcttcaggtacactataactaaccaccaaaatccatgacatttacatgagcactaacaccccccggagcacacgcgtcatgggagacaggatttgcagagagcaacagcccaagaaaaggggcatgagctttgtgaaaattgtgactctgggtggcacatatgtcactgcaaaacttgttgcatgaacagggtcagaatgtgagttggagcaggaggaggaaaatgttggttgggagaacacaatctttggaccactaacagtctctccatcatgtatggtgatctgatcatctcttcctttgtgtggaattgaagtggattcagaaaggggaaaaaaaaagaaagaaaaggagctgccttgcatggcatggcaatggcacctggccctcctcatctttcagggtttcacatatcccaatcaaaggcttaatagcacatctaattgtctaatgataaaggatgatgggaggcaaaagtacatccatgatgatgataataataataatctctggtttttgtatagtaccatgtttttcagctttgagtggcgaaatttgacctcagcgcgacaccgagacgcggtgtactgattgatggcttgttctagctagctgaagtcagagggtgcccttttcttttttttgaactgcagtcagatgatgcttttgattgtctgaaaggatacttagtgttggtgccaaactgcccaatcaagatatgccagggaaaagaaatgatgaagtgcactactttaccctaaattgacttgggaattcaaattaagcatgcagttcttcagtcaacctatgaagtgatcattcatgttcgagaaaaaaatttgtaaaaaataagagtgtttttacgatggtctctaccagcagtattctacgactagtacagatcacaataaaaattgtcaaaatataaacgggtctttttgtttactgaagaagatgtagttgatcattccgaccttgtgtcctgaccggcctgaagtgaagtcttgatggatactgttaagttggtcgatcagttaaaatgaaggtcatctagtacaacaaaataatggctttcattgccaatcaatctattgctggcactactacaattcagaacacaattttaccactcaaattactttttactcactattcatttcatttaaacctataaaaatttagtcgaatatcctccaccgtcatcgtgtcgcgatccgcaatctctctgtatggatagaaggaagaatgttttggatcttagctcacagacttttgtgggcaatttcatccctgtgatatcatgggaagtttcatatgttgacaagggagagtgagcagtgttgacctcaatgatcattcaacatttcctgcctgctggaagccaaaatcttgtccaacaagccgtactccatactgcggctacatatacctctgaaattgctgatcaaatcttcaaaaaaaattgcaagttctacacgcgcttgctcgatccgtcgcggccccataatttttcaccggaaaaaaaatggatgaaaatttttaaccatggtaggacaataaggacccctttgcaagtcatatctgaagctctagaacagatgactgaacattttacatgttgtcaattaattaagctggtcggcagcttaatcgattgatctgaggataattaatcttgtcaataactgttttcacctaaaaacaggtagtagaattctctgatctcacctaaaatccatccagggcaaaaggtagaccagaggataattttgtcagaacatgagatgaaagtaaagggagaagaaaagaaagcatggaaactttgtagtgcagcacttgcaattgcaaagcaagcaaaagggacccaagagcagaagctcatgcttcatggtggcacgtaggccaccctcacagccttcactgccgacgcccggcccgggcccaccgcgcagcctcaccggcctcggacacgtgtacatagagctagtaagcaaaataaaaaataaaaaataaaaaaataaagagagatagaagcttgaactacacatcagtacatcacacacagagagacccttggtgcttactacacatcagtagttagctactggctggtttgttttgtctcctcttcttgcctcccccttataaaggaaggtgagcattgcatcttgctccttagtactcctcctcctgcatcatcatcccaatcctcaattcagaattcagaactgccatttgctaccttacataccatacctagctgaattgctgtcagttaatctcttgatcagatcgatcaccctgcagcttcttgttgtttctagcatttctggtgaagaaaccaaatcttcagcagcttaaggtaggagatcatca

SEQ ID NO: 47 - DNA, 28 bp, Artificial
  Artificially synthesized primer sequence
gcattcactctcccgttcttgatcgctt

SEQ ID NO: 48 - DNA, 28 bp, Artificial
  Artificially synthesized primer sequence
ccggcaaaagaccaactagggacaaacc

SEQ ID NO: 49 - DNA, 26 bp, Artificial
  Artificially synthesized primer sequence
attgccgatccatctacatgagtcaa

SEQ ID NO: 50 - DNA, 27 bp, Artificial
  Artificially synthesized primer sequence
ggtctttggatctcgcacctccaccgc

SEQ ID NO: 51 - DNA, 27 bp, Artificial
  Artificially synthesized primer sequence
ttatgtcagcaatataagcatttctga

SEQ ID NO: 52 - DNA, 23 bp, Artificial
  Artificially synthesized primer sequence
aggctcgatgactgtgctcaacc

SEQ ID NO: 53 - DNA, 27 bp, Artificial
  Artificially synthesized primer sequence
ggtaccctgattcttgcctggcccatg

SEQ ID NO: 54 - DNA, 29 bp, Artificial
  Artificially synthesized primer sequence
ggtaccggtccacaaatgatgtccaattc

SEQ ID NO: 55 - DNA, 27 bp, Artificial
  Artificially synthesized primer sequence
aatgagtagcacgaggactcacccctg

SEQ ID NO: 56 - DNA, 27 bp, Artificial
  Artificially synthesized primer sequence
tctagagctttttgtgagcgtggtgtg

SEQ ID NO: 57 - DNA, 27 bp, Artificial
  Artificially synthesized primer sequence
ccaatatccacaagaaacagaggacaa

SEQ ID NO: 58 - DNA, 27 bp, Artificial
  Artificially synthesized primer sequence
gattctacgtacgtttgtatggatgga

SEQ ID NO: 59 - DNA, 28 bp, Artificial
  Artificially synthesized primer sequence
caaatttcatgtggatggtcctgatcac

SEQ ID NO: 60 - DNA, 27 bp, Artificial
  Artificially synthesized primer sequence
gtccgtacgatgtgctgtacgcactag

SEQ ID NO: 61 - DNA, 27 bp, Artificial
  Artificially synthesized primer sequence
acagtatacactgacttaggtggtgtt

SEQ ID NO: 62 - DNA, 28 bp, Artificial
  Artificially synthesized primer sequence
caatgttgtagagctgcttcgacacaag

SEQ ID NO: 63 - DNA, 28 bp, Artificial
  Artificially synthesized primer sequence
gtcaccactaggtagatcgatcatccct

SEQ ID NO: 64 - DNA, 27 bp, Artificial
  Artificially synthesized primer sequence
gatggcgcgcagcgtcaggtcggtaag

SEQ ID NO: 65 - DNA, 27 bp, Artificial
  Artificially synthesized primer sequence
tatagcttgtgttgcgcacctcgaaag

SEQ ID NO: 66 - DNA, 28 bp, Artificial
  Artificially synthesized primer sequence
cgtgtgagatggatggagatcgtatgac

SEQ ID NO: 67 - DNA, 27 bp, Artificial
  Artificially synthesized primer sequence
tcattcaccatgtgctatggagacaac

SEQ ID NO: 68 - DNA, 26 bp, Artificial
  Artificially synthesized primer sequence
tgctgcaagacctgagtagttcttgg

SEQ ID NO: 69 - DNA, 27 bp, Artificial
  Artificially synthesized primer sequence
aggattttgtgatgggcttggcccaac

SEQ ID NO: 70 - DNA, 27 bp, Artificial
  Artificially synthesized primer sequence
gctcgtcggcaaaagaccaattaggga

SEQ ID NO: 71 - DNA, 28 bp, Artificial
  Artificially synthesized primer sequence
tacaaaggagtccacatcaaccctccag

SEQ ID NO: 72 - DNA, 27 bp, Artificial
  Artificially synthesized primer sequence
gacgatggctaactggtcgtctcagcc

SEQ ID NO: 73 - DNA, 29 bp, Artificial
  Artificially synthesized primer sequence
actctgaataacactgaaacagtccattg

SEQ ID NO: 74 - DNA, 27 bp, Artificial
  Artificially synthesized primer sequence
tgatgatctcctaccttaagctgctga

SEQ ID NO: 75 - DNA, 1314 bp, Oryza sativa
  3′UTR (1)..(1314): 3′UTR of the gene 3
ctgattcttgcctggcccatgcactgcgaccagccatgggacgccgagctcctctgcaagtacctcaaggccggcgtcctcgtgcggccatgggagaagcacaacgaggtcacgccggcgaaggacatccaggaagccatcgaggagggccatgctttctaacggaggagtagccatgcgacaacgtgcacgggagttcggagatgccatccgcgcctccgtggccgccgccggctcgtcggtcgtcgcgcaaagacctggatgacttcgttgcttacatcacgaggtaatcacgtaccgagattgccacgttggaattgatgattctgttctggtaatccaattggctttgccattgggccaggccaccatcttatatctcaaggcccatctggccacgtcgccattccggccataggagcagcagcaggcttccatgtcagccactgctcgatcaatttagtggccctgtttagttgggggcagatcatgaagtaaacttactgaggccttgtttagttggggaaaatttttgggtttgtttgtcacatcggatatacggtatatatttaaagtattaaacgtaatctagtaacaaaacaaattacatattccgtaaggaaactgcgagacaaatttattaagcctaattaataggggtgaaaacgaaacggatattatccgctccgaatccgtccgaagtgaggatatggtaagggtttttagatatccggccggatgcggatgcggatgcggatatggtatcggttatatccggcggatatggattatccgctattttaagcggattatccgataaacgttttggcggataatccgaatttcacagcccatgtaacctctcaatttggcccatttgacacgagcattttcatcgtgtaaacattcagcccatccacgtccaccttatccatcaatctatctaggagttcagtccgtgactccgtcggcgtcgtgtcaaccaattttttcatcgagacttctcttcttccattcctcccacgccgccgcctgacttctccacccagcgccaccgccgccacctgccttctccaccccggcgccgcggcctaccttctccaccccaaagtcacctggttgtctccgtctcggcgccggcgacgcctcctgccatctccagcaccaactctgtcgtatgtgttattatcagaattgaagtctacttctctagatcatctccttctgaccttagctgcatatgtgttattatgtcatatgcaatatgcaacttattagtagtactgtatgagaattggacatcatttgtggacc

SEQ ID NO: 76 - DNA, 22 bp, Artificial
  Artificially synthesized primer sequence
tttagccctgccttcatacgct

SEQ ID NO: 77 - DNA, 20 bp, Artificial
  Artificially synthesized primer sequence
cgtcgttgccgaagaactgg

SEQ ID NO: 78 - DNA, 20 bp, Artificial
  Artificially synthesized primer sequence
tctagaatggccggaagtgg

SEQ ID NO: 79 - DNA, 20 bp, Artificial
  Artificially synthesized primer sequence
gagctcctagttgtagaccc

SEQ ID NO: 80 - DNA, 20 bp, Artificial
  Artificially synthesized primer sequence
tccactgacgtaagggatga

SEQ ID NO: 81 - DNA, 19 bp, Artificial
  Artificially synthesized primer sequence
atcagctcatcgagagcct

SEQ ID NO: 82 - DNA, 23 bp, Artificial
  Artificially synthesized primer sequence
ttgtggatgccctaacagcttgg

SEQ ID NO: 83 - DNA, 18 bp, Artificial
  Artificially synthesized primer sequence
gctattagcttgctttgg

SEQ ID NO: 84 - DNA, 20 bp, Artificial
  Artificially synthesized primer sequence
tccactgacgtaagggatga

SEQ ID NO: 85 - DNA, 19 bp, Artificial
  Artificially synthesized primer sequence
atcagctcatcgagagcct

SEQ ID NO: 86 - DNA, 28 bp, Artificial
  Artificially synthesized primer sequence
gtccgtcaggacattgttggagccgaaa

SEQ ID NO: 87 - DNA, 30 bp, Artificial
  Artificially synthesized primer sequence
gcgtggatatgtcctgcgggtaaatagctg

SEQ ID NO: 88 - DNA, 21 bp, Artificial
  Artificially synthesized primer sequence
tgccaccattcgagttcttca

SEQ ID NO: 89 - DNA, 21 bp, Artificial
  Artificially synthesized primer sequence
ccgaacaacaaaccttgcatg

SEQ ID NO: 90 - DNA, 21 bp, Artificial
  Artificially synthesized primer sequence
cagacctccgtttttgtgcag

SEQ ID NO: 91 - DNA, 20 bp, Artificial
  Artificially synthesized primer sequence
gcgataatgccgtgacgaat

SEQ ID NO: 92 - DNA, 21 bp, Artificial
  Artificially synthesized primer sequence
ggcggataatccgaatttcac

SEQ ID NO: 93 - DNA, 21 bp, Artificial
  Artificially synthesized primer sequence
tggataaggtggacgtggatg

SEQ ID NO: 94 - DNA, 21 bp, Artificial
  Artificially synthesized primer sequence
ggtagacaacactattactcc

SEQ ID NO: 95 - DNA, 19 bp, Artificial
  Artificially synthesized primer sequence
cctgcagtttcaactagac

SEQ ID NO: 96 - DNA, 20 bp, Artificial
  Artificially synthesized primer sequence
cacaattgaactcgtccgga

SEQ ID NO: 97 - DNA, 20 bp, Artificial
  Artificially synthesized primer sequence
tgtacggtttttcacgccac

SEQ ID NO: 98 - DNA, 21 bp, Artificial
  Artificially synthesized primer sequence
aagctgcccaatggaatgttg

SEQ ID NO: 99 - DNA, 21 bp, Artificial
  Artificially synthesized primer sequence
tggaagcaatgtgagtgaccg

SEQ ID NO: 100 - DNA, 21 bp, Artificial
  Artificially synthesized primer sequence
gcacttttttcccaattcccc

SEQ ID NO: 101 - DNA, 21 bp, Artificial
  Artificially synthesized primer sequence
tgaaagcacacggagaccttg

SEQ ID NO: 102 - DNA, 19 bp, Artificial
  Artificially synthesized primer sequence
ttgcacatgccacactcga

SEQ ID NO: 103 - DNA, 21 bp, Artificial
  Artificially synthesized primer sequence
cccgaattcctcttccatgtc

SEQ ID NO: 104 - DNA, 21 bp, Artificial
  Artificially synthesized primer sequence
tccagccccgatcacaatagt

SEQ ID NO: 105 - DNA, 21 bp, Artificial
  Artificially synthesized primer sequence
cggtacgtaatttggcatggc

SEQ ID NO: 106 - DNA, 21 bp, Artificial
  Artificially synthesized primer sequence
tcactgcccttcttgcttttg

SEQ ID NO: 107 - DNA, 20 bp, Artificial
  Artificially synthesized primer sequence
cgccagcacattgttgatgt

SEQ ID NO: 108 - DNA, 20 bp, Artificial
  Artificially synthesized primer sequence
tttcccaccagctcattcca

SEQ ID NO: 109 - DNA, 21 bp, Artificial
  Artificially synthesized primer sequence
tcaccggactcagcaagagaa

SEQ ID NO: 110 - DNA, 20 bp, Artificial
  Artificially synthesized primer sequence
tgctccatgtccaagatgca

SEQ ID NO: 111 - DNA, 21 bp, Artificial
  Artificially synthesized primer sequence
gcagcgcgatgatgtgatact

SEQ ID NO: 112 - DNA, 21 bp, Artificial
  Artificially synthesized primer sequence
agattgcgtctcatttgcctg

SEQ ID NO: 113 - DNA, 21 bp, Artificial
  Artificially synthesized primer sequence
ccgtgttctttctctctgcgt

SEQ ID NO: 114 - DNA, 20 bp, Artificial
  Artificially synthesized primer sequence
gcaagaggtgatgtgctacg

SEQ ID NO: 115 - DNA, 20 bp, Artificial
  Artificially synthesized primer sequence
tcgagctcggtaccctcgtt

SEQ ID NO: 116 - DNA, 20 bp, Artificial
  Artificially synthesized primer sequence
gcaagaggtgatgtgctacg

SEQ ID NO: 117 - DNA, 20 bp, Artificial
  Artificially synthesized primer sequence
agaatcagggtaccctcgtt

SEQ ID NO: 118 - DNA, 20 bp, Artificial
  Artificially synthesized primer sequence
gcaagaggtgatgtgctacg

SEQ ID NO: 119 - DNA, 20 bp, Artificial
  Artificially synthesized primer sequence
gggatcatcgttagctcggg

SEQ ID NO: 120 - DNA, 19 bp, Artificial
  Artificially synthesized primer sequence
gtacgcgtccagaaaagct

SEQ ID NO: 121 - DNA, 18 bp, Artificial
  Artificially synthesized primer sequence
ttggcgaagcgacctctc

SEQ ID NO: 122 - DNA, 19 bp, Artificial
  Artificially synthesized primer sequence
tgccgagatgaggccccga

SEQ ID NO: 123 - DNA, 20 bp, Artificial
  Artificially synthesized primer sequence
ccaccaagggcaagctctac

SEQ ID NO: 124 - DNA, 21 bp, Artificial
  Artificially synthesized primer sequence
agcgctcataacgttcaagga

SEQ ID NO: 125 - DNA, 29 bp, Artificial
  Artificially synthesized primer sequence
agtacgccaccgactcatgtatggacaaa

SEQ ID NO: 126 - DNA, 20 bp, Artificial
  Artificially synthesized primer sequence
gagcctctgttcgtcaagta

SEQ ID NO: 127 - DNA, 20 bp, Artificial
  Artificially synthesized primer sequence
actcgatggtccattaaacc

SEQ ID NO: 128 - DNA, 26 bp, Artificial
  Artificially synthesized primer sequence
ttgtggtgctgatgtctacttgtgtc

SEQ ID NO: 129 - DNA, 22 bp, Artificial
  Artificially synthesized primer sequence
ccctcatcccgaaagaacacta

SEQ ID NO: 130 - DNA, 22 bp, Artificial
  Artificially synthesized primer sequence
tccaccctctccttgagtctct

SEQ ID NO: 131 - DNA, 22 bp, Artificial
  Artificially synthesized primer sequence
gtttaagctgccgatgtgcctg

SEQ ID NO: 132 - DNA, 24 bp, Artificial
  Artificially synthesized primer sequence
gacacgactcatgacacgaacagc

SEQ ID NO: 133 - DNA, 9632 bp, Zea mays
  promoter (1)..(9632): promoter 1 of the gene 12(Zea mays)
cgggatcattgtcggccctttaaccccattgcctcaccaaaaatgtcaattaagagcaaaaaggcaatgagagcataaatatgaacttggaagtgagtacactaataccggagtgcagtggaagtctttgcatcgtccaagttcacctttccctttcaatgcacttttgagactacatcaagtatactcaaacacaaaggttagtctcaaagggtcaagttgtagcatatctccccctaaatatgtgcaccatttgcatatggacttgtgaggtccggggaggtcgtatacaacttgagcaccacaaatatacaacgagtaatgtaaatgcttcaaagtaacatgatcaaaggcatagagcacctgtatgctatagatcaatccaagttacgtgaatctaagacatttagctcactacgcaacctgcaaaaagttttctcatccaacagcttggtaaatatatcggctagctggttgtcggtgctaatatggtacaccttgatatctcccttttgttggtggtctctcaggaagtgatgctggatgtctatgtgcttagtgcggctgtgttcaacaggatgatccgccatgcggattgcactctcattgtcacataggagtgggactttgctcagattgtagccaaagtcccggagggtttgcctcatctaaagtagttgcgcacaacactgtcctgcggcaacatactcggcctcagcggtggatagggcaatggatgtttgtttcttagagctccaggacaccagggaccttcctaaaaattggcacgtccccgatgtgctctttctatcaaccttgcacccggcataatcggagtctgagtatccaatcaagtcaaaggtagaccctttggataccagatcccgaagcaaggtgtagaaactaaatatctaagaattcgcttaacggccacaaggtgacattccttggggtcggattgatatctagaacacatgcatacacttggcataatgtccggtctactagcacataaataaagcaatgaacctatcatggacctgtatgccttttgatcaacagacttacctcctttgttgaggtcaacatgcccgtcggttcccatcggtgtctttgtgggcttggcgtccttcatcccaaaccgcttgagaagatcttgtgtgtactttgtttgggagatgaaggtgtcgtccttgagttgctctacttggaacccaaggaagtaggtcaactcgcccatcatcgatatctcgaacttttgtgtcatcaccctgctaaactcctcacaagacttttgattagtagaaccaaatattatgtcatcaacataaatttggcacacaaaaaagatcaccatcacaagtctttgtaaaaagagtgggatcggctttcccaaccttgaaggcattagcaattaagaaatctctaaggaattcataccatgctcttggggcttgcttaagtccatagagcgccttagagagcttgaacacatggtcggggtacctgtcatcctcaaagccagggggttgttccacgtatacctcctcctttattggcccattgaggaaagcgttcttcacatccatttgaaatagcctgaaagagtggtgagcggcataggctaataaaattcgaatagactctagcctagtcacaggagcaaaagtctcctcgaaatccaaacctacgacttgggcataaccttttgccacaagtcgagcattgtttcttgtcaccaccccgtgctcgtcttgctcttgttgcggaacacccacttggttcccacaacattttgctttagacgtggcaccaggctccaaacttcatttctcttgaagttgttgagctcttcctgcatggccaacacccaatccggatcctgcaaggcctcttctatcctgaaaggatcaatagaagagacaaacgagtaatgctcaccaaagttagctaatcttgagcgagtagttactcccttgcttatgtcacccagaatctggtcaacggggtgatgtcgttggatcgttgctcggacttgagttggaggggcacgtggtccttcttcctccatcacttgttcttcatgtgctcccccttgatcatgtccctcttcttgaggtacctgttcaccgtcttgagttggaggatgcaccattgtggaggaagatggctgatcttgctcctgttgttcctgtggtcgcacatcaccaatcgccatcgtgcgcattgcggccgttggaacatcatcttcatctatgtcatcaagatcaacttgctctcttggagagccattagtctcatcaaatacaacgttgctagagacttcaaccaaacccgatgatttgttgaagactctatacgcctttgtatttgagtcataccctagtaaaaatccttctactgccttgggagcaaatttagaatgtctagctttcttcaccagaatgtaacatttgctcccaaatacacgaaagtaggagacgttgggtttgttaccgataaggagttcgtaggaggtcttcttaagaaggcgatgcagatagagctggtttatggcgtggcaagctatgttcacaacttccgaccaaaaccgctcgggcgtcttgaactctccaagcatcattctcgccatgtcgataagcgtcctgttcttcctctctaccacaccgttttgctgtggtgtgtagggagcggagaactcgtgcttgacgccttcctcctcaagatactcctcaacttgcagattgttgaactcggaggtccatgtgaagaagctccagtggtcttgatgttgtcatcacattcttgctatgatgagtgcttcccacctgtttctctgcctgacatgctgcacaaggcctatctttctcgaaacaaacattggttagtcctaacacatgttctccctttagaagtttatgaaggttcttcatcccaacatgtgctagacgacgatgccacagccagcccatactagtcttagctattaagcatgcatctagatcggtctcctctttcgaaaaatcaactaagtagagtttgtcgtctaatacacccttaaaagctaatgaaccatcactccttctaaagacagatacatcaacgtttgtaaaaagacaattgtaacccatgtgacacaattgacttacagacagaaggttgtaaccaagcaactctactagaaatacattcgatatggagttctcgttggtgagggctatcttgcccaagcctttgaccttgccttggttcccatctccaaagatgatcgtatcatgggaatccttgttcttgacgtaggaggtgaacatcttcttctcccccgtcatgtggtttgtgcatccgttgtcgataatccaacttgagcccccagatgcataaacctgcaaggaatttaagcttgggttttaggtacccaactcttgttgggtcctacaaggttagtcaaaatagtttttggaacccatatgcatggtttgtctcccttgcatttggatcccaacttcctagccactacttttgcattcttacatgaaagaacaaaagaagtgttgcaagcatgaaaaacaacagtaggtttattacacattttcctaggcacatgatgcacaacatgatttttcctaggcctacttctaccatgcacaaaagtagagctagaggcaaatgaaagggaattaggcttacacctagttcctaaataattttggtggttgaattgcccaacacaaatctttggactaactagtttgcccaagtgtatagattatacaggtgtaaaaggctcacactcagccaataaaaagaccaagttttggattcaataaaggagcaaaggggcaaccgagggcacccctggtctggcgcaccggactgtccggtgtgccacaggacagtgaacagtacctgtccggtgcaccaggggactcagactccaactcttcactctcgggaattctcggaagccggcgcgctataattcaccggactgtccggtgtgcaccggacatgtccggtgcgccaacgaaccgcggcctccggaactcgtcatcctcgggttttcacgacagccgctccgctataattcaccggactgtccggtgtgcaccggactgtccggtgtgccagcggagcaacggctctctgcggcgccaacggctccctgcgcagcattaaatgcgcgcgcagcgcgcgcagacgtcagggctgcccataccgatgcaccggacatcaaacagtgcatgtccggtgtgcaccggacacccaggcgggcccacaagtcagaagctccaacggctagaatccaacggcagtggtgacgtggcaggggcaccggactgtccggtgtgcaccggactgtccggtgcgccatcgaacagaagcctccagccaacggtcaagtttggtggttggggctataaataccccaaccaccccacattcattgccatccaagttttccaacttctaaccacttacaagagctaggcattcaattctagacacatacaaagagatcaaatcctctccaattccactcaagcctttagtgactagcgagagagatttgccgtgttcttttgagctcttgcgcttggatcgcattctttctttctcttttgctcttgtgatcaacactcaattgtaaccgaggcaagaggcaccaattgtgtggtggcccttgcgggaaagttttgttcccagttgattgagaagaaggaaagctcactcggtccgagggaccgtttgagagagggaagggttgaaagagacccggcctttgtggcctcctcaacggggagtaggtttgcaagaaccgaacctcggtaaaacaaatccacgtgtcactctctttacttgcttgcgatttgttttgcgccctctcttgcggactcatttattattactaacgctaacaccgacttgtagttgggattatttttgtaaatttcagtttcgccctattcaccccccctctaggcgactatcagcaaacatggcatgtggatcaatgtaagtagtatgaacataactcttattataaatggaatgactagcaattttcctatcataaataaaagcatgattcctttgaggactactagccataggggcattccctttctccttgttgacaacgggagccttttggcttgttaagttcttggtttcctttcaaaacccaagtccatccttaattgaggggtgtctaccaatagtgtaggcatctctagcaactttaatttatcaaaatcacttttgcaagtcttaagttgagcattaagacttgccacctcatcattcaatttagtaatagcaataagatgttcatcacatgcatcaacatcaaagtctttacatctattacaaataacaacatgctatacacatgaactagatttattaacttcctctagcttagcatttaattcatcatttaaactccttaaactagaaacagattcatggcaagcagacaactcagaggatagcatttcatttcttttaatttctagagcaagagatttttgaacactaataaatttgtcatgttcttcatataaaatatcctcttgcttttctaaaagtctatccttttcatttagagcatcaatcaatttcattaattttctctactttggctctatctaaacctttaaataaactagagtaatctacttcatcatcggaggattcttcatcactagatgaagtgtacttgggtgtgtcctgaacacgtacctttttctccttggccataagacatgtatgacgttcgttgggaagagtgaggatttgttgaaggccgaggtagcaagtccttcgtcgtcgaagtcggaagaggagcagttcgagtcccattcctttccaaggtgcacctcaccctttgccttcctgtagttcttctttttctccttcttcccgctcttctcttgtccctggtcactatcattatcgggacaatttgcaataaagtgaccagtcttaccgcatttgaagcatgagtgttttccctttgttttatttctgttggggtactccttgcgtcctttgagtgcggtcttgaagcgcttgatgataagcgccatctcatcttcattgagtccggcagcctccacttgagctaccttgcttggtagcgcctccttgctgcttgttgctttgagagcaacgggctgcggctcgtagatgggtactggaccgttcaaggcgtcatccacatatcgtgcctcctttaccatcatgcgcccgctcacaaattttccgagtatctcctcgggcgtcatcttggtgtacctggggttatcacgaataagattgacaagatggggtcaattacggtaaatgacctgagcatgaatcggacgacatcatggtccgtccatcttgtgcttccatagcttcgaatcttgttgaccagggtcttgagcatattgtaggtttgtgttggctcttctcccctaatcatggcgaatctccccagctcaccttccaccaactccattttggtaatcatagtggcgtcgtttccctcatgagatatcttgagggtgtcccagatttgcttggcgttgtctaagccacttactttgttatactcatccctgcaaagtgaagctagcaagacagtagtagcttgggcattttttatgaatttgttcatttatcatcacagggttatccgtactatcgaaatgcattccatttcccacaatttcccaaatgttaggatggagagaaaatagatgactatgcattttatgactccaaaatgagtaatcctctccatcaaagtgtgggggttttccaagaggaatagagagtaaatgggcatttgaattgtacggaatacgggaataatcaaaggaatagttttgattaaccatctttttctttgacgaagaatcatcatcgtcgtcgtctcttggtgaagaagaagatgcatcgctgtcgtagtagatgatcttcttgatgcgcctcttcttctccccgtccttcttcttgtgactcaagcctgagtcagtgggcttgtcatctcttgactcgttgaagatggactccttctccttgtcgttgaccaccatcctctttcccttaggatccatctcttcgggcgataagtccattagatgaagagtacgactctgataccaattgagagcacctagagggggtgaataggtgatcctgtaaaatcaaacactaaatagccacaaaacttagttatagaagtgttagtgtgactaagtagctttgaagaaagttcttgtgaacacaataatcacagagagagcaacacaagagacacgtggtttttattccgtggttcagccaagtgacacttgcctacttccacgttgtggcgtcccaatggacgagggttgcactcaacccctttcaagtgatccaatgatcaacttgaataccacgactttcttacttatagtctttctcccatttgcaaggaatctccacaagttggagcctctcgcccttacaataaagatcacaaagaaggcacaagagtaaggctaggagagcaacacacacacaacacacaaatctgcagcacacacacgcacacaagccaagacttgagctcgaaacgtagcacagagagttcacaactcgaatggagctcaaatcactaacacaatctatcaaatgcgcggaggcggagtgtgagtcttagaatgcttagtgaatgcttgggtgactcctccatgcgcctaggggtcccttttatagccccaaggcacctaggagccgttgaagacaaacttggaaggtcatccttgccttctgtcgagtggcgcaccggacagtccggtgcgccactggacaactactgtagcctgtccggtgcgtgatttccttccaaatcgggcacagatgaccgttgcagctccgggcccgttggcgcaccggacactgtccggtgcacactggacagtccggtgccccccgccaaccgttggagctgacacgccttttcaccggagaccagcctagcgcaccggacactgtccggtgcaccaccggacagtcctgtgtgccagtccgagctgaagtttggctgctcacaaccaagtcttttgcaatctgattcctctcttttcggcactgtttctagcacttagacaaacaatgttagttcacaaaaacaatgtactaagtctataaacgtaccttttactttgatttgcatatttcacacatttagcatattagtactgaaataatgtgttgggcatctaatcaccaaaatacttatagaaattgcccaagggcacatttccctttcacttatgcctcacgtcatggaatagaagcatacatatagacagattaataaagaattagagatgtttgaatgcattaaagctaatagttaattgactgaaaattgttagtgaaattagctagctaacaaatagctagctaactattagttaatttactaaaagaggttaatatctcaactattaactatactgttttgatgtctttagctaattttaacagctaattattagctctagtgtattcatacaccctttaattagcaaagtgatccgacataatgttaacattttttcaaaatttaaacgtggagacaacatggaaccaaaacaacttgtgctttcatatagtacgaaaataaactattatattgtactgcaaatttacacgtttggaggccctaaatatttagggccctgttccagtgcaaagctcatacaccctctggcacggggctggtggtactcgataaagaatgttttaccgagtgtccgataaaatatactcggtaaagcgcaaattcccaacactcggcaaaaatagccgagggtggttacgccttgcgtggcactcgactattgatcgctgccactgtcagcgacgcgaggtgcggccggtcatgcctacgtcgccccggcttgcgtcatcagctccacgcaacccatgccggccggcagcgcgccgtgcatgtcgttacgtagtaccacatgtgtgcaccgaatcattttattagtcgttactcaggaggacgtggctcgctgcctagactggctctgtttcaatctgctatggctatgccttaacacttgatagcaagctctacctttatattttttactgctgagtaatagtaagaatgtgtgtgtgtgtgtggttcatataaataaatatgtaaattcctctgtggactgtagatctccatatgtaggccataggcaatctagctagctgtcccggccgcggcccgccacaataaatatattgtccgttggggccaccattagttgagctctcgtacgtccactctttttttttctcataataacaatcttctggtccatatgttcctgtatgtacacacgtgcatgtaaagtgatgtatgcttgctctcaactgaactactgaagtcaggtcttgtatatgcccactgcacctgcctgcgcctatgcgtgcatgtgggttcttgccactgttactggcttcttatatctagaccagtagccgccacttctgcctctttttataccagtcccggtgctgcggaacacacagagcgaggtatctagagacagagagggctgtgtggagagagagagagagagagatcg

SEQ ID NO: 134 - DNA, 30 bp, Artificial
  Artificially synthesized primer sequence
cgggatcattgtcggccctttaaccccatt

SEQ ID NO: 135 - DNA, 50 bp, Artificial
  Artificially synthesized primer sequence
cgatctctctctctctctctctccacacagccctctctgtctctagatac

SEQ ID NO: 136 - DNA, 30 bp, Artificial
  Artificially synthesized primer sequence
cagaaggttgtaaccaagcaactctactag

SEQ ID NO: 137 - DNA, 6480 bp, Zea mays
  promoter (1)..(6480): promoter 2 of the gene 12(Zea mays)
cagaaggttgtaaccaagcaactctactagaaatacattcgatatggagttctcgttggtgagggctatcttgcccaagcctttgaccttgccttggttcccatctccaaagatgatcgtatcatgggaatccttgttcttgacgtaggaggtgaacatcttcttctcccccgtcatgtggtttgtgcatccgttgtcgataatccaacttgagcccccagatgcataaacctgcaaggaatttaagcttgggttttaggtacccaactcttgttgggtcctacaaggttagtcaaaatagtttttggaacccatatgcatggtttgtctcccttgcatttggatcccaacttcctagccactacttttgcattcttacatgaaagaacaaaagaagtgttgcaagcatgaaaaacaacagtaggtttattacacattttcctaggcacatgatgcacaacatgatttttcctaggcctacttctaccatgcacaaaagtagagctagaggcaaatgaaagggaattaggcttacacctagttcctaaataattttggtggttgaattgcccaacacaaatctttggactaactagtttgcccaagtgtatagattatacaggtgtaaaaggctcacactcagccaataaaaagaccaagttttggattcaataaaggagcaaaggggcaaccgagggcacccctggtctggcgcaccggactgtccggtgtgccacaggacagtgaacagtacctgtccggtgcaccaggggactcagactccaactcttcactctcgggaattctcggaagccggcgcgctataattcaccggactgtccggtgtgcaccggacatgtccggtgcgccaacgaaccgcggcctccggaactcgtcatcctcgggttttcacgacagccgctccgctataattcaccggactgtccggtgtgcaccggactgtccggtgtgccagcggagcaacggctctctgcggcgccaacggctccctgcgcagcattaaatgcgcgcgcagcgcgcgcagacgtcagggctgcccataccgatgcaccggacatcaaacagtgcatgtccggtgtgcaccggacacccaggcgggcccacaagtcagaagctccaacggctagaatccaacggcagtggtgacgtggcaggggcaccggactgtccggtgtgcaccggactgtccggtgcgccatcgaacagaagcctccagccaacggtcaagtttggtggttggggctataaataccccaaccaccccacattcattgccatccaagttttccaacttctaaccacttacaagagctaggcattcaattctagacacatacaaagagatcaaatcctctccaattccactcaagcctttagtgactagcgagagagatttgccgtgttcttttgagctcttgcgcttggatcgcattctttctttctcttttgctcttgtgatcaacactcaattgtaaccgaggcaagaggcaccaattgtgtggtggcccttgcgggaaagttttgttcccagttgattgagaagaaggaaagctcactcggtccgagggaccgtttgagagagggaagggttgaaagagacccggcctttgtggcctcctcaacggggagtaggtttgcaagaaccgaacctcggtaaaacaaatccacgtgtcactctctttacttgcttgcgatttgttttgcgccctctcttgcggactcatttattattactaacgctaacaccgacttgtagttgggattatttttgtaaatttcagtttcgccctattcaccccccctctaggcgactatcagcaaacatggcatgtggatcaatgtaagtagtatgaacataactcttattataaatggaatgactagcaattttcctatcataaataaaagcatgattcctttgaggactactagccataggggcattccctttctccttgttgacaacgggagccttttggcttgttaagttcttggtttcctttcaaaacccaagtccatccttaattgaggggtgtctaccaatagtgtaggcatctctagcaactttaatttatcaaaatcacttttgcaagtcttaagttgagcattaagacttgccacctcatcattcaatttagtaatagcaataagatgttcatcacatgcatcaacatcaaagtctttacatctattacaaataacaacatgctatacacatgaactagatttattaacttcctctagcttagcatttaattcatcatttaaactccttaaactagaaacagattcatggcaagcagacaactcagaggatagcatttcatttcttttaatttctagagcaagagatttttgaacactaataaatttgtcatgttcttcatataaaatatcctcttgcttttctaaaagtctatccttttcatttagagcatcaatcaatttcattaattttctctactttggctctatctaaacctttaaataaactagagtaatctacttcatcatcggaggattcttcatcactagatgaagtgtacttgggtgtgtcctgaacacgtacctttttctccttggccataagacatgtatgacgttcgttgggaagagtgaggatttgttgaaggccgaggtagcaagtccttcgtcgtcgaagtcggaagaggagcagttcgagtcccattcctttccaaggtgcacctcaccctttgccttcctgtagttcttctttttctccttcttcccgctcttctcttgtccctggtcactatcattatcgggacaatttgcaataaagtgaccagtcttaccgcatttgaagcatgagtgttttccctttgttttatttctgttggggtactccttgcgtcctttgagtgcggtcttgaagcgcttgatgataagcgccatctcatcttcattgagtccggcagcctccacttgagctaccttgcttggtagcgcctccttgctgcttgttgctttgagagcaacgggctgcggctcgtagatgggtactggaccgttcaaggcgtcatccacatatcgtgcctcctttaccatcatgcgcccgctcacaaattttccgagtatctcctcgggcgtcatcttggtgtacctggggttatcacgaataagattgacaagatggggtcaattacggtaaatgacctgagcatgaatcggacgacatcatggtccgtccatcttgtgcttccatagcttcgaatcttgttgaccagggtcttgagcatattgtaggtttgtgttggctcttctcccctaatcatggcgaatctccccagctcaccttccaccaactccattttggtaatcatagtggcgtcgtttccctcatgagatatcttgagggtgtcccagatttgcttggcgttgtctaagccacttactttgttatactcatccctgcaaagtgaagctagcaagacagtagtagcttgggcattttttatgaatttgttcatttatcatcacagggttatccgtactatcgaaatgcattccatttcccacaatttcccaaatgttaggatggagagaaaatagatgactatgcattttatgactccaaaatgagtaatcctctccatcaaagtgtgggggttttccaagaggaatagagagtaaatgggcatttgaattgtacggaatacgggaataatcaaaggaatagttttgattaaccatctttttctttgacgaagaatcatcatcgtcgtcgtctcttggtgaagaagaagatgcatcgctgtcgtagtagatgatcttcttgatgcgcctcttcttctccccgtccttcttcttgtgactcaagcctgagtcagtgggcttgtcatctcttgactcgttgaagatggactccttctccttgtcgttgaccaccatcctctttcccttaggatccatctcttcgggcgataagtccattagatgaagagtacgactctgataccaattgagagcacctagagggggtgaataggtgatcctgtaaaatcaaacactaaatagccacaaaacttagttatagaagtgttagtgtgactaagtagctttgaagaaagttcttgtgaacacaataatcacagagagagcaacacaagagacacgtggtttttattccgtggttcagccaagtgacacttgcctacttccacgttgtggcgtcccaatggacgagggttgcactcaacccctttcaagtgatccaatgatcaacttgaataccacgactttcttacttatagtctttctcccatttgcaaggaatctccacaagttggagcctctcgcccttacaataaagatcacaaagaaggcacaagagtaaggctaggagagcaacacacacacaacacacaaatctgcagcacacacacgcacacaagccaagacttgagctcgaaacgtagcacagagagttcacaactcgaatggagctcaaatcactaacacaatctatcaaatgcgcggaggcggagtgtgagtcttagaatgcttagtgaatgcttgggtgactcctccatgcgcctaggggtcccttttatagccccaaggcacctaggagccgttgaagacaaacttggaaggtcatccttgccttctgtcgagtggcgcaccggacagtccggtgcgccactggacaactactgtagcctgtccggtgcgtgatttccttccaaatcgggcacagatgaccgttgcagctccgggcccgttggcgcaccggacactgtccggtgcacactggacagtccggtgccccccgccaaccgttggagctgacacgccttttcaccggagaccagcctagcgcaccggacactgtccggtgcaccaccggacagtcctgtgtgccagtccgagctgaagtttggctgctcacaaccaagtcttttgcaatctgattcctctcttttcggcactgtttctagcacttagacaaacaatgttagttcacaaaaacaatgtactaagtctataaacgtaccttttactttgatttgcatatttcacacatttagcatattagtactgaaataatgtgttgggcatctaatcaccaaaatacttatagaaattgcccaagggcacatttccctttcacttatgcctcacgtcatggaatagaagcatacatatagacagattaataaagaattagagatgtttgaatgcattaaagctaatagttaattgactgaaaattgttagtgaaattagctagctaacaaatagctagctaactattagttaatttactaaaagaggttaatatctcaactattaactatactgttttgatgtctttagctaattttaacagctaattattagctctagtgtattcatacaccctttaattagcaaagtgatccgacataatgttaacattttttcaaaatttaaacgtggagacaacatggaaccaaaacaacttgtgctttcatatagtacgaaaataaactattatattgtactgcaaatttacacgtttggaggccctaaatatttagggccctgttccagtgcaaagctcatacaccctctggcacggggctggtggtactcgataaagaatgttttaccgagtgtccgataaaatatactcggtaaagcgcaaattcccaacactcggcaaaaatagccgagggtggttacgccttgcgtggcactcgactattgatcgctgccactgtcagcgacgcgaggtgcggccggtcatgcctacgtcgccccggcttgcgtcatcagctccacgcaacccatgccggccggcagcgcgccgtgcatgtcgttacgtagtaccacatgtgtgcaccgaatcattttattagtcgttactcaggaggacgtggctcgctgcctagactggctctgtttcaatctgctatggctatgccttaacacttgatagcaagctctacctttatattttttactgctgagtaatagtaagaatgtgtgtgtgtgtgtggttcatataaataaatatgtaaattcctctgtggactgtagatctccatatgtaggccataggcaatctagctagctgtcccggccgcggcccgccacaataaatatattgtccgttggggccaccattagttgagctctcgtacgtccactctttttttttctcataataacaatcttctggtccatatgttcctgtatgtacacacgtgcatgtaaagtgatgtatgcttgctctcaactgaactactgaagtcaggtcttgtatatgcccactgcacctgcctgcgcctatgcgtgcatgtgggttcttgccactgttactggcttcttatatctagaccagtagccgccacttctgcctctttttataccagtcccggtgctgcggaacacacagagcgaggtatctagagacagagagggctgtgtggagagagagagagagagagatcg

SEQ ID NO: 138 - DNA, 36 bp, Artificial
  Artificially synthesized primer sequence
taccgagctcgaattctgcagcgtgacccggtcgtg

SEQ ID NO: 139 - DNA, 30 bp, Artificial
  Artificially synthesized primer sequence
agtttaaactgaattcccgatctagtaaca

SEQ ID NO: 140 - DNA, 13830 bp, Artificial
  pKLB525/UbiGhd7/GateHd3a
cgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggcaattcgcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgtcgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaattaaggactagtctggtgacgatgaaagtggcagatggagatgaggtgagccgaggagcagcagagccagtgttcgtacgagagccaaagagcagacgagagccgaagttgagttcagggccgttcctggctttttaggggcccagtgcgaaattcgatatagaggccccatccacacacaaatatatataattatacatatatcaccgaatatttgacgtataaaaattattatgcactattttaaagtttataagataacagatataaaatatagcaaaaaacacttacttgttatatgatattattaaaaatatcttctaatattttgtgatacaaagtcatcgcttatatcatcgagatcaatctcatccaacaacttttttcgatatatagaatcgccaagccatttaacctctcttgagttattgttgaccataaatagttcataaaaattttaattttacaaagcttctgtctaccgaagcaaccatcatatgtatagtttataatactctctctatatcaaaataaaatttgttttgcttctttagtggatttatcatcatcaatataaacataaaaactaagagctaaaccaaataccattttgaaacggatggagtattcgataagtaattgagacattagtttaacaatattaacgagactagtcgataactcgatagcacatcgttttttgcgtatttatgtttagaatagatagatttaacaaaaaaatgttagcgttcagtggctaggggggctctattttttgggcccggtgcggccgcacccacggcacccctcagggccggccctggttgagttggtaatagagctaagcgatgtaatgcatacgataaaagaatgaccacgtgtatgcgtctcaagcaaggtccgctcttctgagaacgtgcggacctaccgtcaataattttctaccctttcccactccgtgccaggtgccaccctccccaagccctcgcgccgcctccgagacagccgcccgcaaccatggccaccgccgccaccgcggccgccgcgctcaccggcgccactaccgctacgcccaagtcgaggcgccgagcccaccacttggccacccggcgcgccctcgccgcgcccatcaggtgctcagcgttgtcacgcgccacgccgacggctcccccggccactccgctacgtccgtggggccccaacgagccccgcaagggctccgacatcctcgtcgaggctctcgagcgctgtggcgtccgtgacgtcttcgcctaccccggcggcgcatccatggagatccaccaggcactcacccgctcccccgtcatcgccaaccacctcttccgccacgaacaaggggaggccttcgccgcctccggctacgcgcgctcctcgggccgcgttggcgtctgcatcgccacctccggccccggcgccaccaacctagtctctgcgctcgcagacgcgttgctcgactccgtccccatggtcgccatcacgggacaggtgccgcgacgcatgattggcaccgacgcctttcaggagacgcccatcgtcgaggtcacccgctccatcaccaagcacaactacctggtcctcgacgtcgacgacatcccccgcgtcgtgcaggaggccttcttcctcgcatcctctggtcgcccggggccggtgcttgttgacatccccaaggacatccagcagcagatggcggtgccggcctgggacacgcccatgagtctgcctgggtacatcgcgcgccttcccaagcctcccgcgactgaatttcttgagcaggtgctgcgtcttgttggtgaatcacggcgccctgttctttatgttggcggtggctgtgcagcatcaggtgaggagttgtgccgctttgtggagttgactggaatcccagtcacaactactcttatgggccttggcaacttccccagcgacgacccactgtcactgcgcatgcttggtatgcatggcacagtgtatgcaaattatgcagtggataaggccgatctgttgcttgcatttggtgtgcggtttgatgatcgtgtgacagggaaaattgaggcttttgcaggcagagctaagattgtgcacattgatattgatcctgctgagattggcaagaacaagcagccacatgtgtccatctgtgcagatgttaagcttgctttgcagggcatgaatactcttctggaaggaagcacatcaaagaagagctttgacttcggctcatggcatgatgaattggatcagcaaaagagggagtttccccttggatataaaatcttcaatgaggaaatccagccacaatatgctattcaggttcttgatgagttgacgaagggggaggccatcattgccacaggtgttgggcagcaccagatgtgggcggcacagtattacacttacaagcggccaaggcagtggctgtcttcagctggtcttggggctatgggatttggtttgccggctgctgctggtgctgctgtggccaacccaggtgtcactgttgttgacatcgacggagatggtagcttcctcatgaacattcaggagctagctatgatccgtattgagaacctcccagtcaaggtctttgtgctaaacaaccagcacctcgggatggtggtgcagttggaggacaggttctataaggccaatagagcacacacattcttgggaaacccagagaacgaaagtgagatatatccagattttgtggcaattgctaaagggttcaacattccagcagtccgtgtgacaaagaagagcgaagtccatgcagcaatcaagaagatgcttgaggctccagggccgtacctcttggatataatcgtcccgcaccaggagcatgtgttgcctatgatccctattggtggggctttcaaggatatgatcctggatggtgatggcaggactgtgtattgatctaaagttcagcatgcactgcctacctgcctatctttgacatgcatgagctagtacaagtgtgatatgtttttatcgatgtgatggtactctgttatggtaatcttaagtagcatccaaccctgtgtgtagtatgttgtttccgtgttggcatatgtttcagaagccatcatgtaagtgccttttactacatataaataaggtaataagcattgttatgcactggttctgaattggtcttcttttgccaaatataggtcctgtttgatacctatagctctagaaaatttggtgtagaaaatttggtgtggttggtggagcaggtcattaggtgttccaagatctaggccttctagaggatcctcgcgatccggacttaagatttaaatggtaccgagctcgaattctgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaagcggggatcctctagagtcgacctgcaggcatgcaagctagcttacaagtttgtacaaaaaagcaggctttaaaggaaccaattcagtcgactggatccatgtatccatacgatgttccagattatgctgtcggcgccggttggtctcatcctcaatttgaaaaaggaggcgccatgtcgatgggaccagcagccggagaaggatgtggcctgtgcggcgccgacggtggcggctgttgctcccgccatcgccacgatgatgatggattccccttcgtcttcccgccgagtgcgtgccaggggatcggcgccccggcgccaccggtgcacgagttccagttcttcggcaacgacggcggcggcgacgacggcgagagcgtggcctggctgttcgatgactacccgccgccgtcgcccgttgctgccgccgccgggatgcatcatcggcagccgccgtacgacggcgtcgtggcgccgccgtcgctgttcaggaggaacaccggcgccggcgggctcacgttcgacgtctccctcggcgaacggcccgacctggacgccgggctcggcctcggcggcggcggcggccggcacgccgaggccgcggccagcgccaccatcatgtcatattgtgggagcacgttcactgacgcagcgagctcgatgcccaaggagatggtggccgccatggccgatgatggggagagcttgaacccaaacacggtggttggcgcaatggtggagagggaggccaagctgatgaggtacaaggagaagaggaagaagaggtgctacgagaagcaaatccggtacgcgtccagaaaagcctatgccgagatgaggccccgagtgagaggtcgcttcgccaaagaacctgatcaggaagctgtcgcaccgccatccacctatgtcgatcctagtaggcttgagcttggacaatggttcagatagaattcgcggccgcactcgagatatctagacccagctttcttgtacaaagtggtgatactagtcccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcaagcttcgacctcgagacaagtttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgccgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagtggtctcgagggccataagggcctctagaatggccggaagtggcagggacagggaccctcttgtggttggtagggttgtgggtgatgtgctggacgcgttcgtccggagcaccaacctcaaggtcacctatggctccaagaccgtgtccaatggctgcgagctcaagccgtccatggtcacccaccagcctagggtcgaggtcggcggcaatgacatgaggacattctacacccttgtgatggtagacccagatgcaccaagcccaagtgaccctaaccttagggagtatctacattggttggtcactgatattcctggtactactgcagcgtcatttgggcaagaggtgatgtgctacgagagcccaaggccaaccatggggatccaccggctggtgttcgtgctgttccagcagctggggcgtcagacagtgtacgcgcccgggtggcgtcagaacttcaacaccaaggacttcgccgagctctacaacctcggctcgccggtcgccgccgtctacttcaactgccagcgcgaggccggctccggcggcaggagggtctacaactagggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacgtcaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaccgcggacctaggtgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccgaacgttctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctactagt

SEQ ID NO: 141 - DNA, 13931 bp, Artificial
  pKLB525/UbiGhd7/GateAdh5′UTRHd3a
cgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggcaattcgcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgtcgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaattaaggactagtctggtgacgatgaaagtggcagatggagatgaggtgagccgaggagcagcagagccagtgttcgtacgagagccaaagagcagacgagagccgaagttgagttcagggccgttcctggctttttaggggcccagtgcgaaattcgatatagaggccccatccacacacaaatatatataattatacatatatcaccgaatatttgacgtataaaaattattatgcactattttaaagtttataagataacagatataaaatatagcaaaaaacacttacttgttatatgatattattaaaaatatcttctaatattttgtgatacaaagtcatcgcttatatcatcgagatcaatctcatccaacaacttttttcgatatatagaatcgccaagccatttaacctctcttgagttattgttgaccataaatagttcataaaaattttaattttacaaagcttctgtctaccgaagcaaccatcatatgtatagtttataatactctctctatatcaaaataaaatttgttttgcttctttagtggatttatcatcatcaatataaacataaaaactaagagctaaaccaaataccattttgaaacggatggagtattcgataagtaattgagacattagtttaacaatattaacgagactagtcgataactcgatagcacatcgttttttgcgtatttatgtttagaatagatagatttaacaaaaaaatgttagcgttcagtggctaggggggctctattttttgggcccggtgcggccgcacccacggcacccctcagggccggccctggttgagttggtaatagagctaagcgatgtaatgcatacgataaaagaatgaccacgtgtatgcgtctcaagcaaggtccgctcttctgagaacgtgcggacctaccgtcaataattttctaccctttcccactccgtgccaggtgccaccctccccaagccctcgcgccgcctccgagacagccgcccgcaaccatggccaccgccgccaccgcggccgccgcgctcaccggcgccactaccgctacgcccaagtcgaggcgccgagcccaccacttggccacccggcgcgccctcgccgcgcccatcaggtgctcagcgttgtcacgcgccacgccgacggctcccccggccactccgctacgtccgtggggccccaacgagccccgcaagggctccgacatcctcgtcgaggctctcgagcgctgtggcgtccgtgacgtcttcgcctaccccggcggcgcatccatggagatccaccaggcactcacccgctcccccgtcatcgccaaccacctcttccgccacgaacaaggggaggccttcgccgcctccggctacgcgcgctcctcgggccgcgttggcgtctgcatcgccacctccggccccggcgccaccaacctagtctctgcgctcgcagacgcgttgctcgactccgtccccatggtcgccatcacgggacaggtgccgcgacgcatgattggcaccgacgcctttcaggagacgcccatcgtcgaggtcacccgctccatcaccaagcacaactacctggtcctcgacgtcgacgacatcccccgcgtcgtgcaggaggccttcttcctcgcatcctctggtcgcccggggccggtgcttgttgacatccccaaggacatccagcagcagatggcggtgccggcctgggacacgcccatgagtctgcctgggtacatcgcgcgccttcccaagcctcccgcgactgaatttcttgagcaggtgctgcgtcttgttggtgaatcacggcgccctgttctttatgttggcggtggctgtgcagcatcaggtgaggagttgtgccgctttgtggagttgactggaatcccagtcacaactactcttatgggccttggcaacttccccagcgacgacccactgtcactgcgcatgcttggtatgcatggcacagtgtatgcaaattatgcagtggataaggccgatctgttgcttgcatttggtgtgcggtttgatgatcgtgtgacagggaaaattgaggcttttgcaggcagagctaagattgtgcacattgatattgatcctgctgagattggcaagaacaagcagccacatgtgtccatctgtgcagatgttaagcttgctttgcagggcatgaatactcttctggaaggaagcacatcaaagaagagctttgacttcggctcatggcatgatgaattggatcagcaaaagagggagtttccccttggatataaaatcttcaatgaggaaatccagccacaatatgctattcaggttcttgatgagttgacgaagggggaggccatcattgccacaggtgttgggcagcaccagatgtgggcggcacagtattacacttacaagcggccaaggcagtggctgtcttcagctggtcttggggctatgggatttggtttgccggctgctgctggtgctgctgtggccaacccaggtgtcactgttgttgacatcgacggagatggtagcttcctcatgaacattcaggagctagctatgatccgtattgagaacctcccagtcaaggtctttgtgctaaacaaccagcacctcgggatggtggtgcagttggaggacaggttctataaggccaatagagcacacacattcttgggaaacccagagaacgaaagtgagatatatccagattttgtggcaattgctaaagggttcaacattccagcagtccgtgtgacaaagaagagcgaagtccatgcagcaatcaagaagatgcttgaggctccagggccgtacctcttggatataatcgtcccgcaccaggagcatgtgttgcctatgatccctattggtggggctttcaaggatatgatcctggatggtgatggcaggactgtgtattgatctaaagttcagcatgcactgcctacctgcctatctttgacatgcatgagctagtacaagtgtgatatgtttttatcgatgtgatggtactctgttatggtaatcttaagtagcatccaaccctgtgtgtagtatgttgtttccgtgttggcatatgtttcagaagccatcatgtaagtgccttttactacatataaataaggtaataagcattgttatgcactggttctgaattggtcttcttttgccaaatataggtcctgtttgatacctatagctctagaaaatttggtgtagaaaatttggtgtggttggtggagcaggtcattaggtgttccaagatctaggccttctagaggatcctcgcgatccggacttaagatttaaatggtaccgagctcgaattctgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaagcggggatcctctagagtcgacctgcaggcatgcaagctagcttacaagtttgtacaaaaaagcaggctttaaaggaaccaattcagtcgactggatccatgtatccatacgatgttccagattatgctgtcggcgccggttggtctcatcctcaatttgaaaaaggaggcgccatgtcgatgggaccagcagccggagaaggatgtggcctgtgcggcgccgacggtggcggctgttgctcccgccatcgccacgatgatgatggattccccttcgtcttcccgccgagtgcgtgccaggggatcggcgccccggcgccaccggtgcacgagttccagttcttcggcaacgacggcggcggcgacgacggcgagagcgtggcctggctgttcgatgactacccgccgccgtcgcccgttgctgccgccgccgggatgcatcatcggcagccgccgtacgacggcgtcgtggcgccgccgtcgctgttcaggaggaacaccggcgccggcgggctcacgttcgacgtctccctcggcgaacggcccgacctggacgccgggctcggcctcggcggcggcggcggccggcacgccgaggccgcggccagcgccaccatcatgtcatattgtgggagcacgttcactgacgcagcgagctcgatgcccaaggagatggtggccgccatggccgatgatggggagagcttgaacccaaacacggtggttggcgcaatggtggagagggaggccaagctgatgaggtacaaggagaagaggaagaagaggtgctacgagaagcaaatccggtacgcgtccagaaaagcctatgccgagatgaggccccgagtgagaggtcgcttcgccaaagaacctgatcaggaagctgtcgcaccgccatccacctatgtcgatcctagtaggcttgagcttggacaatggttcagatagaattcgcggccgcactcgagatatctagacccagctttcttgtacaaagtggtgatactagtcccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcaagcttcgacctcgagacaagtttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgccgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagtggtctcgagggccataagggcctctagagaattccaagcaacgaactgcgagtgattcaagaaaaaagaaaacctgagctttcgatctcttcggagtggtttcttgttctttgaaaaagagggggattaatggccggaagtggcagggacagggaccctcttgtggttggtagggttgtgggtgatgtgctggacgcgttcgtccggagcaccaacctcaaggtcacctatggctccaagaccgtgtccaatggctgcgagctcaagccgtccatggtcacccaccagcctagggtcgaggtcggcggcaatgacatgaggacattctacacccttgtgatggtagacccagatgcaccaagcccaagtgaccctaaccttagggagtatctacattggttggtcactgatattcctggtactactgcagcgtcatttgggcaagaggtgatgtgctacgagagcccaaggccaaccatggggatccaccggctggtgttcgtgctgttccagcagctggggcgtcagacagtgtacgcgcccgggtggcgtcagaacttcaacaccaaggacttcgccgagctctacaacctcggctcgccggtcgccgccgtctacttcaactgccagcgcgaggccggctccggcggcaggagggtctacaactagggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacgtcaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaccgcggacctaggtgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccgaacgttctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctactagt

SEQ ID NO: 142 - DNA, 26 bp, Artificial
  Artificially synthesized primer sequence
ctgtggactgtagatctccatatgta

(19) United States
(12) Patent Application Publication
(10) Pub. No.: US 2016/0138032 A1
(43) Pub. Date: May 19, 2016

(54) POACEAE PLANT WHOSE FLOWERING TIME IS CONTROLLABLE

(71) Applicants: INCORPORATED ADMINISTRATIVE AGENCY NATIONAL AGRICULTURE AND FOOD RESEARCH ORGANIZATION, Tsukuba-shi, Ibaraki (JP); NATIONAL INSTITUTE OF AGROBIOLOGICAL SCIENCES, Tsukuba-shi, Ibaraki (JP)

(72) Inventors: Takeshi IZAWA, Tsukuba-shi (JP); Ryo OKADA, Tsukuba-shi (JP); Naokuni ENDO, Tsukuba-shi (JP); Yasue NEMOTO, Tsukuba-shi (JP); Tadashi TAKAMIZO, Nasushiobara-shi (JP); Shoko TSUZUKI, Toyota-shi (JP); Satoru NISHIMURA, Toyota-shi (JP)

(73) Assignees: NATIONAL INSTITUTE OF AGROBIOLOGICAL SCIENCES, Tsukuba-shi, Ibaraki (JP); INCORPORATED ADMINISTRATIVE AGENCY NATIONAL AGRICULTURE AND FOOD RESEARCH ORGANIZATION, Tsukuba-shi, Ibaraki (JP)

(21) Appl. No.: 14/772,838

(22) PCT Filed: Mar. 4, 2014
(86) PCT No.: PCT/JP2014/055507

§ 371 Date: Nov. 20, 2015

(30) Foreign Application Priority Data
Mar. 4, 2013 (JP) 2013-042009

(51) Int. Cl. C12N 15/82; A01H 5/10

(52) U.S. Cl. CPC C12N 15/827; A01H 5/10

(57) ABSTRACT

It has been found that introducing into a Poaceae plant an Hd3a gene, which is a flower-bud-formation inducing gene, positioned downstream of a promoter whose expression is induced by a plant activator treatment makes it possible to control the flowering time of the Poaceae plant in accordance with a plant activator treatment timing. It has been found that further introducing a Ghd7 gene, which functions to suppress flower bud formation, into the plant makes it possible to suppress the expression of an endogenous Hd3a gene and increase the efficiency of controlling the flowering time.

### TECHNICAL FIELD

The present invention relates to a Poaceae plant whose flowering time is controllable by a plant activator treatment. More specifically, the present invention relates to a Poaceae plant comprising an Hd3a gene, which is a flower-bud-formation inducing gene, introduced and positioned downstream of a promoter sensitive to a plant activator.

### BACKGROUND ART

Flowering is a very important event involved in plant propagation. In agricultural production, the flowering time of a crop is one of major traits determining the yield. Since each cultivar exhibits its own environmental response based on the genetic background, the flowering time is also a factor limiting the region and season suitable for cultivation of the cultivar. From the opposite point of view, once a cultivar to be cultivated and the cultivation timing are set, the flowering time and the harvesting time at the location are automatically determined, and the yield is also roughly determined at the same time. In this manner, the flowering-time trait of crops has been actively studied and is an important breeding objective in cultivar improvements.

Conventionally, the flowering time of a plant is modified through selection of early maturing and late maturing lines in hybrid progenies obtained by crossing, selection from mutant lines obtained by inducing an artificial mutation using a mutagenic agent or radiation, and so forth. However, these methods require a lot of time and effort, and also have a problem that the direction and the degree of the mutation are unpredictable, and other problems. Moreover, recently, many genes for controlling flowering (flowering control genes) have been isolated, and these genes are reported to be utilizable in the flowering time regulation. For rice (Oryza sativa), an Hd3a gene, an RFT1 gene, and the like encoding florigen (flowering hormone) have been isolated as flowering promoter genes, and a Ghd7 gene (Lhd4 gene) and the like have been isolated as flowering suppressor genes. Methods for modifying the flowering time by introducing these flowering control genes or inhibiting the functions of endogenous flowering control genes have been presented (PTLs 1, 2, and 3; NPLs 1 and 2). For example, it is known that a transgenic rice that incorporates a DNA cassette containing a Ghd7 gene positioned downstream of a CaMV35S promoter, which is a constitutive expression promoter, does not flower even after 100 days (PTL 3). Further, it is also known that transforming normal rice with a DNA cassette containing an Hd3a gene positioned downstream of a CaMV35S promoter can produce a plant which flowers earlier than the parental line (NPL 1). Such methods are advantageous in that it is possible to obtain a target plant in a relatively short period with high reliability. All of the above-described methods make it possible to obtain a plant whose flowering time is changed so that the plant can flower earlier or later than the parental line. Nevertheless, the flowering time of lines produced by any of the methods is determined by characters (not environmental response and the like, but excessive expression, ectopic expression, and the like) of the introduced genes and cannot be altered freely. Additionally, there is a report that the existence of florigen in an amount more than necessary in rice causes very small inflorescences (NPL 3). If a plant is transformed with a DNA cassette containing an Hd3a gene positioned downstream of a CaMV35S promoter or the like constitutively expressing the gene at a high level, it is highly likely that, as a result of the constitutive and excessive expressions of the Hd3a gene, the flowering with only very small inflorescences starts at an abnormally early stage in comparison with normal flowering time.

On the other hand, there has also been an attempt to induce an Hd3a gene expression by a heat shock stimulation, the Hd3a gene being positioned downstream of an HSP promoter, which is an inducible promoter (NPL 4). Nevertheless, in this experiment, the heading was observed also under non-inducible conditions. This is presumably due to the Hd3a gene expression at a low level under the non-inducible conditions.

Meanwhile, there have been various reports on plant activators that are chemicals for increasing the resistance of a plant to diseases, specifically, inductions of gene expressions of resistance related genes, and promoters of the resistance related genes activated by actions of the plant activators (PTLs 4, 5, and 6). However, there is no example where a promoter sensitive to a plant activator is used to control the flowering time of a plant.

### CITATION LIST

### Patent Literatures

[PTL 1] Japanese Unexamined Patent Application Publication No. 2002-153283
[PTL 2] Japanese Unexamined Patent Application Publication No. 2004-89036
[PTL 3] Japanese Unexamined Patent Application Publication No. 2004-290190
[PTL 4] Japanese Unexamined Patent Application Publication No. Hei 9-270

### Non Patent Literatures

[NPL 1] Kojima et al. Plant Cell Physiol. 2002; 43 (10): 1096-105
[NPL 2] Xue et al., Nat Genet. 2008; 40 (6): 761-7
[NPL 3] Izawa et al., Genes Dev. 2002; 16 (15): 2006-20
[NPL 4] Endo-Higashi and Izawa 2011; Plant Cell Physiol. 2011 52 (6): 1083-94
[NPL 5] Shimono et al., Plant Cell 2007; 19: 2064-2076
[NPL 6] Umemura et al., Plant J. 2009; 57: 463-472

### SUMMARY OF INVENTION

### Technical Problem

The present invention has been made in view of the problems of the above-described conventional techniques. An object of the present invention is to provide a Poaceae plant whose flowering time is controllable by an artificial flower bud induction at a certain timing.

### Solution to Problem

In order to achieve the above object, the present inventors have earnestly studied. As a result, the inventors have found that introducing an Hd3a gene, which is a flower-bud-formation inducing gene, positioned downstream of a promoter sensitive to a plant activator into a Poaceae plant makes it possible to control the flowering time of the Poaceae plant in accordance with a plant activator treatment timing. In this regard, the present inventors have also successfully isolated a novel promoter for ensuring the gene expression suitable for controlling the flowering time of a Poaceae plant from a transcriptome analysis of rice having been subjected to a plant activator treatment in a field.

Moreover, the present inventors have found that further introducing a Ghd7 gene, which functions to suppress flower bud formation, into the Poaceae plant makes it possible to suppress the expression of an endogenous Hd3a gene and increase the efficiency of controlling the flowering time. A Ghd7 protein is known to suppress the expression of the endogenous Hd3a gene, and the above result means that a Ghd7 protein does not suppress the activity of an Hd3a protein. From the foregoing, the present invention has also revealed that a Ghd7 protein can be utilized in combination with an artificially expressed Hd3a protein in controlling the flowering time of a Poaceae plant

The present invention is based on these findings, and more specifically relates to the following inventions.

(1) A Poaceae plant whose flowering time is controllable by a plant activator treatment, the Poaceae plant comprising an expression construct in which an Hd3a gene is ligated downstream of a promoter sensitive to a plant activator.

(2) The Poaceae plant according to (1), wherein the plant activator is any one of probenazole and isotianil.

(3) The Poaceae plant according to (2), wherein the promoter is a DNA of any one of (a) to (c) below:

(a) a DNA having a nucleotide sequence of any one of SEQ ID NOs: 1, 133, and 137;

(b) a DNA having a nucleotide sequence of any one of SEQ ID NOs: 1, 133, and 137 in which one or more nucleotides are substituted, deleted, added, and/or inserted, the DNA having an activity of the promoter sensitive to the plant activator; and

(c) a DNA having a nucleotide sequence having a homology of 70% or more with the nucleotide sequence of any one of SEQ ID NOs: 1, 133, and 137, the DNA having the activity of the promoter sensitive to the plant activator.

(4) The Poaceae plant according to any one of (1) to (3), further comprising an expression construct of a gene encoding a protein that suppresses an expression of an endogenous Hd3a gene but does not suppress an activity of an Hd3a protein.

(5) The Poaceae plant according to (4), wherein the protein that suppresses the expression of the endogenous Hd3a gene but does not suppress the activity of the Hd3a protein is a Ghd7 protein.

(6) The Poaceae plant according to (4) or (5), wherein the gene encoding the protein that suppresses the expression of the endogenous Hd3a gene but does not suppress the activity of the Hd3a protein is ligated downstream of a constitutive expression promoter.

(7) The Poaceae plant according to (6), wherein the constitutive expression promoter is a corn-derived ubiquitin promoter.

(8) A Poaceae plant, which is any one of a progeny and a clone of the Poaceae plant according to any one of (1) to (7).

(9) A propagation material of the Poaceae plant according to any one of (1) to (8).

(10) A method for producing a Poaceae plant whose flowering time is controllable by a plant activator treatment, the method comprising the step of introducing into a Poaceae plant cell an expression construct in which an Hd3a gene is ligated downstream of a promoter sensitive to a plant activator, and regenerating the plant.

(11) A method for inducing flowering of a Poaceae plant, the method comprising the step of treating the Poaceae plant according to any one of (1) to (8) with the plant activator.

(12) An agent for inducing flowering of the Poaceae plant according to any one of (1) to (8), the agent comprising the plant activator as an active ingredient.

### Advantageous Effects of Invention

The flowering time of the plant produced by the present invention can be flexibly controlled, although such control is impossible by the conventional techniques. Thus, it is possible to induce the flower bud formation of the plant at an optimal timing for the harvest in accordance with a cultivation environment (cultivation location, cultivation timing), a genetic background, an intended use, and so forth.

### BRIEF DESCRIPTION OF DRAWINGS

FIG. 1 is a figure of the flowering (heading) time of a Ghd7-gene constitutively expressing line (Ghd7ox). (A) is a bar graph illustrating the flowering time of the Ghd7ox in a T0 generation grown in a glass greenhouse. Numbers at the bottom of the graph indicate independent T0 individuals, and “Cont.” indicates a control line having been transformed with only the vector. (B) is a photograph showing amounts of a Ghd7 protein accumulated in the Ghd7ox. (C) is a photograph of a membrane stained with Ponceau S after the Ghd7 protein detection in B.

FIG. 2 is a drawing illustrating flowering-time control plasmids. (A) illustrates a configuration of pRiceFOX/Ubi:Ghd7/Gate:Hd3a. (B) illustrates a configuration of pRiceFOX/Ubi: Ghd7/Gate:Adh5′UTR: Hd3a HPT: a hygromycin resistance gene, Ghd7: a Ghd7 cDNA, Hd3a: a Kasalath cultivar Hd3a cDNA, P35S: a cauliflower mosaic virus 35S promoter, PUbi: a corn-derived ubiquitin promoter, Tg7: a g7 terminator, Tnos: a nos terminator, ADH5′UTR: OsADH2 5′UTR, and RB and LB: sequences at right and left borders of T-DNA.

FIG. 3 is a figure illustrating the heading time of rice transformants in which exogenously introduced Hd3a was expressed using different promoters in a background where the Ghd7 gene was constitutively expressed. The bar graph is the result of examining the heading time of the transgenic lines in the T0 generation transferred to a glass greenhouse. Numbers at the bottom of the bar graph indicate independent T0 individuals. The table at the bottom shows the presence or absence of the introduced genes in each line, and +/− indicates the presence or absence of the exogenously introduced Ghd7 cDNA or Hd3a cDNA.

FIG. 4 shows graphs illustrating the number of genes which were observed to have the expression changed by a spray treatment with two plant activators (Oryzemate and Routine) in a field. (A) illustrates the number of genes whose expression level was increased 2-fold or more (white) or decreased to ½ or less (black) by spraying the chemicals. (B) illustrates the number of genes whose expression level was increased 10-fold or more (white) or decreased to 1/10 or less (black) by spraying the chemicals.

FIG. 5 shows graphs illustrating the expression data from a microarray analysis on plant-activator inducible genes or SAR related genes.

FIG. 6 shows graphs illustrating the expression data from the microarray analysis on flowering-time control related genes.

FIG. 7A shows graphs illustrating the expression data on genes (1) to (6) selected from the microarray analysis.

FIG. 7B shows graphs illustrating the expression data on genes (7) to (12) selected from the microarray analysis.

FIG. 7C shows graphs illustrating the expression data on a gene (13) selected from the microarray analysis.

FIG. 8 shows graphs illustrating the expression data from a quantitative RT-PCR analysis on transformants obtained using promoters of the genes (2), (4), and (5). The transformants were grown in a growth chamber (long-day conditions: 14.5 hours of the light period: 9.5 hours of the dark period, temperature setting: 28° C. during the light period: 25° C. during the dark period, illumination: a metal-halide lamp of 500 μE). (A) illustrates levels of exogenously introduced Hd3a expressed. (B) illustrates levels of endogenous expressions of the candidate genes themselves utilizing the promoters.

FIG. 9 shows graphs illustrating the expression data from the quantitative RT-PCR analysis on transformants obtained by using promoters of the genes (6), (7), (9), (10), and (13). The transformants were grown in a glass greenhouse. (A) illustrates levels of exogenously introduced Hd3a expressed. (B) illustrates expression levels of the candidate genes like those in FIG. 8.

FIG. 10 shows graphs of the expression analyses (A, B, D, E, F) and flowering examination (C) on transformants obtained by using a promoter of the gene (3). The transformants were grown in the growth chamber (long-day conditions: 14.5 hours of the light period: 9.5 hours of the dark period, temperature setting: 28° C. during the light period: 25° C. during the dark period, illumination: a metal-halide lamp of 500 μE).

FIG. 11 shows graphs of the expression analyses (A, B) and flowering examination (C) on transformants obtained by using a promoter of the gene (12). The transformants were grown in the glass greenhouse. Numbers on the horizontal axis indicate independent T0 individuals. C1 to C4 indicate independent T0 individuals into which only the vector was introduced. #31 in the T0 lines is a line which was observed to have a flag leaf appeared but did not reach the heading stage. (C) illustrates days when flag leaves were observed.

FIG. 12 shows graphs of the expression analyses (A, B, C) on the transformants obtained by using the promoter of the gene (12). The transformants were grown in the glass greenhouse. Numbers on the horizontal axis indicate the independent T0 individuals. C1 to C4 indicate the independent T0 individuals into which only the vector was introduced.

FIG. 13 shows photographs of flowering-induced lines obtained by using the promoter of the gene (12). (A) is a photograph of a T0-41 line produced in Example 4. The plant flowered (produced ears) on Day 65 after the Oryzemate granule treatment. (B) is a photograph showing the vicinity of the ears of the T0-41 line. (C) is a photograph of a T0-30 line in Example 4 shown in FIG. 11C. The plant flowered on Day 45 after the Routine granule treatment. (D) is a photograph showing the vicinity of the ears of the T0-30 line. In both of the lines, individuals only flowered when subjected to the plant activator treatment.

FIG. 14 shows graphs illustrating the expression analysis using leaf samples of the transformants shown in FIG. 11 on Week 12 after the chemical treatment.

FIG. 15 shows graphs of a morphological examination on the heads of the transformants obtained by using the promoter of the gene (12). Examined were (A) the number of grains per head, (B) the number of primary rachis branches/head, (C) the average number of grains/primary rachis branch, and (D) the ear length of each head on a culm of the line in Example 4 shown in FIG. 11C.

FIG. 16 shows graphs of the morphological examination on the heads of the transformants obtained by using the promoter of the gene (12). Examined were the number of grains per head, the number of primary rachis branches/head, the average number of grains/primary rachis branch, and the ear length of all the heads harvested from the line in Example 4 shown in FIG. 11C. (A) is a scattergram of the number of primary rachis branches/head against the number of grains per head, (C) is a scattergram of the average number of grains/primary rachis branch against the number of grains per head, and (E) is a scattergram of the ear length against the number of grains per head. (B), (D), and (F) show the corresponding average values and standard deviations of (A), (C), and (E), respectively. “Cont. (untreated)” had n=27, “(12) T0 line (untreated)” had n=29, “Cont. (treated)” had n=26, and “(12) T0 line (treated)” had n=33.

FIG. 17 is a figure of the flowering induction test on progenies of the transformants obtained by using the promoter of the gene (12). (A) shows the flowering status in the T0 generation of the lines used in the progeny test. (B) shows the result of the genomic Southern blotting analysis. (C) shows the flowering status of the inbred progenies of the transformants with the introduced gene segregated. The numbers at the bottom of the graph indicate line names of the T1 generations.

FIG. 18 is a figure of the flowering induction test in a field. (A) is a graph illustrating the flowering status in a chemical test in the field. In the middle of the figure (INTRODUCED GENE), the presence or absence of the introduced gene is indicated. The black circle indicates an individual confirmed to have the introduced gene by the PCR analysis. The white circle indicates an individual expected to have the introduced gene. X indicates an individual not expected to have the introduced gene. (B) is a photograph showing the result of the genomic PCR analysis.

FIG. 19 shows graphs of the flowering induction test on a transformant T0 generation (transformation generation) obtained by using the flowering-time control DNA cassette in which the translational enhancer was introduced. The promoter of the gene (12) was introduced into the flowering-time control plasmid shown in FIG. 2B to produce the transformants, and the plant-activator-agent treatment test was conducted. The numbers at the bottom of each graph indicate line names of the T0 generation. (A) illustrates levels of exogenously introduced Hd3a expressed. (B) illustrates levels of the endogenous gene (12) expressed. (C) illustrates days until the heading after the chemical treatment. The T0 lines #14 and 20 are lines which were observed to have a flag leaf appeared but did not reach the heading stage. (C) illustrates days when flag leaves were observed.

FIG. 20 shows graphs of the flowering induction test on the transformants obtained by using the flowering-time control DNA cassette in which the translational enhancer was introduced. The promoter of the gene (12) was introduced into the flowering-time control plasmid shown in FIG. 2B to produce the transformants, and the plant-activator-agent treatment test was conducted. (A) illustrates levels of Ghd7 expressed. (B) illustrates levels of endogenous Hd3a expressed. Numbers on the horizontal axis indicate independent T0 individuals. C1 and C2 indicate independent T0 individuals into which only the vector was introduced.

FIG. 21 shows photographs of the flowering-induced lines obtained by using the translational enhancer (the transformants of Example 8 shown in FIG. 19C). (A) is a photograph of the T0-8 line. The plant flowered (produced ears) on Day 38 after the Routine granule treatment. (B) is a photograph showing the vicinity of the ears of the T0-8 line. (C) is a photograph of the T0-24 line. The plant flowered on Day 35 after the Routine granule treatment. (D) is a photograph showing the vicinity of the ears of the T0-24 line. In both of the lines, individuals only flowered when subjected to the plant activator treatment.

FIG. 22 shows graphs of the flowering induction test on transformants with genetic backgrounds of feed rice cultivars. The transformants with the genetic backgrounds of the feed rice cultivars Tachisugata and Kitaaoba were produced using the promoter of the gene (12), and the plant-activator-agent treatment test was conducted. (A) illustrates levels of exogenously introduced Hd3a expressed. (B) illustrates levels of the gene (12) expressed. (C) illustrates days until the heading after the tillers were transferred. “Tachisugata 1” and so forth or “Kitaaoba 1” and so forth at the bottom of each graph indicate independent T0 individuals obtained by transforming Tachisugata or Kitaaoba with the flowering-time control plasmid. “Tachisugata C1”, “Tachisugata C2”, and “Kitaaoba C1” indicate control individuals each having been transformed with only the vector. Kitaaoba 5 in the T0 line is a line which was observed to have a flag leaf appeared but did not reach the heading stage. (C) illustrates days when flag leaves were observed.

FIG. 23 shows graphs of the flowering induction test on the transformants with the genetic background of the feed rice cultivars. The transformants with the genetic backgrounds of the feed rice cultivars Tachisugata and Kitaaoba were produced using the promoter of the gene (12), and the plant-activator-agent treatment test was conducted. (A) illustrates levels of Ghd7 expressed. (B) illustrates levels of endogenous Hd3a expressed. (C) illustrates levels of OsMADS14 expressed.

FIG. 24 is a figure of a re-flowering induction test. Tillers of the untreated individuals of the line described in Example 8 (the T0-24 line shown in FIG. 19C) which had not flowered were divided again for the treatment/the untreatment, and the plant-activator-agent treatment test was conducted again. Photographs of the test lines are shown at the top, and a schematic drawing of the experimental method for the re-flowering induction test is shown at the bottom.

FIG. 25 is a graph illustrating the flowering status of the line described in Example 4 (the T0-30 line shown in FIG. 11C) and the two lines described in Example 8 (the T0-8 line and the T0-24 line shown in FIG. 19C) in the re-flowering induction test.

FIG. 26 shows graphs illustrating the plant-activator induction of an orthologous gene of the rice the gene (12) in corn. (A) is represented by the real axis. (B) is represented by the logarithmic axis. Each value is shown with the average value and the standard deviation from three independent samples.

FIG. 27 shows schematic drawings of vector constructs for corn.

FIG. 28 is a drawing illustrating flowering-time control plasmids used to transform corn. (A) illustrates a configuration of pKLB525/Ubi:Ghd7/Gate:Hd3a. (B) illustrates a configuration of pKLB525/Ubi:Ghd7/Gate:Adh5′UTR:Hd3a. ZmALS: a corn-derived two-point mutated ALS-inhibiting herbicide resistance gene, Ghd7: a Ghd7 cDNA, Hd3a: a Kasalath cultivar Hd3a cDNA, PZmALS: a corn-derived ALS promoter, PZmUbi: a corn-derived ubiquitin promoter, TALS: a corn-derived ALS terminator, Tnos: a nos terminator, ADH5′UTR: OsADH2 5′UTR, and RB and LB: sequences at right and left borders of T-DNA.

FIG. 29 is a schematic drawing of transformation vector constructs for corn. (A) illustrates a configuration of a vector construct in which each of the rice gene (12) promoter (SEQ ID NO: 1) and two corn gene (12)-ortholog promoters (SEQ ID NOs: 133 and 137) was to be incorporated into pKLB525/Ubi:Ghd7/Gate:Hd3a. (B) illustrates a configuration of a vector construct in which each of the rice-derived gene (12) promoter (SEQ ID NO: 1) and the two corn-derived gene (12)-ortholog promoters (SEQ ID NOs: 133 and 137) was to be incorporated into pKLB525/Ubi:Ghd7/Gate:Adh5rUTR:Hd3a.

FIG. 30A is a graph illustrating the expression data on exogenously introduced Hd3a in the chemical induction test on corn transformants (T0 individuals) obtained by using the rice gene (12) promoter (SEQ ID NO: 1) or the corn gene (12)-ortholog promoter (SEQ ID NO: 133). VC indicates a vector control, and Mi29 indicates a wild type corn line.

FIG. 30B is a graph illustrating the endogenous-expression data on the gene (12) ortholog of corn in the chemical induction test on the corn transformants (T0 individuals) obtained by using the rice gene (12) promoter (SEQ ID NO: 1) or the corn gene (12)-ortholog promoter (SEQ ID NO: 133). VC indicates the vector control, and Mi29 indicates the wild type corn line.

FIG. 31 is a schematic drawing of a rice transformation vector construct in which the corn-derived gene (12)-ortholog promoters were to be used. Illustrated is a configuration of the vector construct in which each of the corn-derived gene (12)-ortholog promoters (SEQ ID NOs: 133 and 137) was to be incorporated into pRiceFOX/Ubi:Ghd7/Gate:Hd3a (FIG. 2A).

FIG. 32A is a graph illustrating the expression data on exogenously introduced Hd3a in the chemical induction test on rice transformants (T0 individuals) obtained by using the corn gene (12)-ortholog promoters (SEQ ID NOs: 133 and 137). C1 and C2 indicate vector controls. The data on lines provided with asterisks show the analysis results each obtained using a single individual by collecting leaves before and after the treatment without performing division.

FIG. 32B is a graph illustrating the expression data on the gene (12) in the chemical induction test on the rice transformants (T0 individuals) obtained by using the corn gene (12)-ortholog promoters (SEQ ID NOs: 133 and 137). C1 and C2 indicate the vector controls. The data on lines provided with asterisks show the analysis results each obtained using a single individual by collecting leaves before and after the treatment without performing division.

FIG. 33 is a graph illustrating levels of the exogenously introduced Ghd7 gene expressed in leaf samples collected before the chemical treatment in the chemical induction test on sugarcane transformants (T0 individuals) obtained by using the rice gene (12) promoter (SEQ ID NO: 1). Numbers on the horizontal axis of the graph indicate independent T0 individuals. “12GH” and “Q165 (WT)” indicate control individuals.

FIG. 34 is a graph of the chemical induction test on the sugarcane transformants (T0 individuals) obtained by using the rice gene (12) promoter (SEQ ID NO: 1). The sugarcane transformants were treated with the chemical (treated plot) or treated with only water (untreated plot). After 16 days, leaf samples were collected. The graph illustrates levels of the exogenously introduced Hd3a gene expressed. Numbers on the horizontal axis of the graph indicate the independent T0 individuals. “12GH” and “Q165 (WT)” indicate the control individuals. Note that FIG. 34 shows the experimental result of each individual of the transformants and so forth. Four individuals were prepared by division from one individual of the transgenic plants, and these individuals were separated into two individuals as the treatment individuals, and two individuals as the untreatment individuals for the experiment.

FIG. 35A is a graph of the flowering induction test on transformants of the feed rice cultivar Kitaaoba as the background cultivar. The transformants with the Kitaaoba genetic background were produced using the promoter of the gene (12), and the flowering induction test was conducted using the plant activator agent. The graph illustrates levels of the exogenously introduced Hd3a gene expressed in leaf samples collected on Day 3 after the chemical treatment. Numbers on the horizontal axis at the bottom of the graph indicate independent T0 individuals. “C1” and “C2” indicate control individuals each having been transformed with only the vector.

FIG. 35B is a graph of the flowering induction test on the transformants of the feed rice cultivar Kitaaoba as the background cultivar. The transformants with the Kitaaoba genetic background were produced using the promoter of the gene (12), and the flowering induction test was conducted using the plant activator agent. The graph illustrates levels of the gene (12) expressed in the leaf samples collected on Day 3 after the chemical treatment. Numbers on the horizontal axis at the bottom of the graph indicate the independent T0 individuals. “C1” and “C2” indicate the control individuals each having been transformed with only the vector.

FIG. 35C is a graph of the flowering induction test on the transformants of the feed rice cultivar Kitaaoba as the background cultivar. The transformants with the Kitaaoba genetic background were produced using the promoter of the gene (12), and the flowering induction test was conducted using the plant activator agent. Illustrated are days until the heading after the tillers were transferred. Numbers on the horizontal axis at the bottom of the graph indicate the independent T0 individuals. “C1” and “C2” indicate the control individuals each having been transformed with only the vector.

FIG. 36A is a graph illustrating levels of the exogenously introduced Ghd7 gene expressed in the leaf samples collected on Day 3 after the chemical treatment in the flowering induction test on the transformants of the feed rice cultivar Kitaaoba as the background cultivar. Numbers on the horizontal axis at the bottom of the graph indicate the independent T0 individuals. “C1” and “C2” indicate the control individuals each having been transformed with only the vector.

FIG. 36B is a graph illustrating levels of the endogenous Hd3a gene expressed in the leaf samples collected on Day 3 after the chemical treatment in the flowering induction test on the transformants of the feed rice cultivar Kitaaoba as the background cultivar. Numbers on the horizontal axis at the bottom of the graph indicate the independent T0 individuals. “C1” and “C2” indicate the control individuals each having been transformed with only the vector.

FIG. 37A is a graph illustrating the result of analyzing levels of the exogenously introduced Hd3a gene expressed in leaf samples of the transformants of the Kitaaoba background cultivar shown in FIGS. 35 and 36 on Week 2 after the chemical treatment. Numbers on the horizontal axis at the bottom of the graph indicate the independent T0 individuals. “C1” and “C2” indicate the control individuals each having been transformed with only the vector.

FIG. 37B is a graph illustrating the result of analyzing levels of the gene (12) expressed in the leaf samples of the transformants of the Kitaaoba background cultivar shown in FIGS. 35 and 36 on Week 2 after the chemical treatment. Numbers on the horizontal axis at the bottom of the graph indicate the independent T0 individuals. “C1” and “C2” indicate the control individuals each having been transformed with only the vector.

FIG. 37C is a graph illustrating the result of analyzing levels of the exogenously introduced Ghd7 gene expressed in the leaf samples of the transformants of the Kitaaoba background cultivar shown in FIGS. 35 and 36 on Week 2 after the chemical treatment. Numbers on the horizontal axis at the bottom of the graph indicate the independent T0 individuals. “C1” and “C2” indicate the control individuals each having been transformed with only the vector.

FIG. 37D is a graph illustrating the result of analyzing levels of the endogenous Hd3a gene expressed in the leaf samples of the transformants of the Kitaaoba background cultivar shown in FIGS. 35 and 36 on Week 2 after the chemical treatment. Numbers on the horizontal axis at the bottom of the graph indicate the independent T0 individuals. “C1” and “C2” indicate the control individuals each having been transformed with only the vector.

FIG. 38A is a graph of the flowering induction test on transformants of the feed rice cultivar Tachisugata as the background cultivar. The transformants with the Tachisugata genetic background were produced using the promoter of the gene (12), and the flowering induction test was conducted using the plant activator agent. The graph illustrates levels of the exogenously introduced Hd3a gene expressed in leaf samples collected on Day 5 after the chemical treatment. Numbers on the horizontal axis at the bottom of the graph indicate independent T0 individuals. “C1” and “C2” indicate control individuals each having been transformed with only the vector.

FIG. 38B is a graph of the flowering induction test on the transformants of the feed rice cultivar Tachisugata as the background cultivar. The transformants with the Tachisugata genetic background were produced using the promoter of the gene (12), and the flowering induction test was conducted using the plant activator agent. The graph illustrates levels of the gene (12) expressed in the leaf samples collected on Day 5 after the chemical treatment. Numbers on the horizontal axis at the bottom of the graph indicate the independent T0 individuals. “C1” and “C2” indicate the control individuals each having been transformed with only the vector.

FIG. 38C is a graph of the flowering induction test on the transformants of the feed rice cultivar Tachisugata as the background cultivar. The transformants with the Tachisugata genetic background were produced using the promoter of the gene (12), and the flowering induction test was conducted using the plant activator agent. The graph illustrates days until the heading after the tillers were transferred. Numbers on the horizontal axis at the bottom of the graph indicate the independent T0 individuals. “C1” and “C2” indicate the control individuals each having been transformed with only the vector.

FIG. 39A is a graph illustrating levels of the exogenously introduced Ghd7 gene expressed in the leaf samples collected on Day 5 after the chemical treatment in the flowering induction test on the transformants of the feed rice cultivar Tachisugata as the background cultivar. Numbers on the horizontal axis at the bottom of the graph indicate the independent T0 individuals. “C1” and “C2” indicate the control individuals each having been transformed with only the vector.

FIG. 39B is a graph illustrating levels of the endogenous Hd3a gene expressed in the leaf samples collected on Day 5 after the chemical treatment in the flowering induction test on the transformants of the feed rice cultivar Tachisugata as the background cultivar. Numbers on the horizontal axis at the bottom of the graph indicate the independent T0 individuals. “C1” and “C2” indicate the control individuals each having been transformed with only the vector.

FIG. 40A is a graph illustrating the result of analyzing the exogenously introduced Hd3a gene expressed in leaf samples of the transformants of the Tachisugata background cultivar shown in FIGS. 38 and 39 on Week 2 after the chemical treatment. Numbers on the horizontal axis at the bottom of the graph indicate the independent T0 individuals. “C1” and “C2” indicate the control individuals each having been transformed with only the vector.

FIG. 40B is a graph illustrating the result of analyzing the gene (12) expressed in the leaf samples of the transformants of the Tachisugata background cultivar shown in FIGS. 38 and 39 on Week 2 after the chemical treatment. Numbers on the horizontal axis at the bottom of the graph indicate the independent T0 individuals. “C1” and “C2” indicate the control individuals each having been transformed with only the vector.

FIG. 40C is a graph illustrating the result of analyzing the exogenously introduced Ghd7 gene expressed in the leaf samples of the transformants of the Tachisugata background cultivar shown in FIGS. 38 and 39 on Week 2 after the chemical treatment. Numbers on the horizontal axis at the bottom of the graph indicate the independent T0 individuals. “C1” and “C2” indicate the control individuals each having been transformed with only the vector.

FIG. 40D is a graph illustrating the result of analyzing the endogenous Hd3a gene expressed in the leaf samples of the transformants of the Tachisugata background cultivar shown in FIGS. 38 and 39 on Week 2 after the chemical treatment. Numbers on the horizontal axis at the bottom of the graph indicate the independent T0 individuals. “C1” and “C2” indicate the control individuals each having been transformed with only the vector.

### DESCRIPTION OF EMBODIMENTS

The present invention provides a Poaceae plant whose flowering time is controllable by a plant activator treatment, the Poaceae plant comprising an expression construct in which an Hd3a gene is ligated downstream of a promoter to be activated by an action of a plant activator.

In the present invention, the term “plant activator” means a chemical that exhibits an effect of controlling a disease not by directly acting on the pathogen but by increasing the resistance of a plant to the disease. A plant activator does not have a direct microbial activity, and hence has advantages in safety to the environment because resistant pathogens hardly occur, and period during which the effect by one treatment lasts.

As the plant activator used in the present invention, for example, any one of probenazole and isotianil can be suitably used, but the plant activator is not limited thereto. Oryzemate (Meiji Seika Kaisha, Limited) is known as a commercial agrochemical containing probenazole as an ingredient. Moreover, Routine (Bayer CropScience AG) is known as a commercial agrochemical containing isotianil as an ingredient. In the present invention, when a Poaceae plant is to be treated with these plant activators, the plant activators may be in the form of these agrochemicals in the treatment.

Moreover, in the present invention, the “promoter sensitive to a plant activator” means a promoter activated by an action of a plant activator so that an expression of the gene ligated downstream of the promoter can be induced. The promoter is not particularly limited, but is preferably one that strongly suppresses an expression of the Hd3a gene ligated downstream thereof when not induced (state where the plant activator treatment is not performed), and that allows the Hd3a gene ligated downstream thereof to be expressed at an appropriate site and an appropriate level when induced (state where the plant activator treatment is performed). It is known that the expression of a florigen gene such as the Hd3a gene is suppressed during the vegetative growth but is dramatically induced when conditions such as day length are satisfied (Suarez et al., Nature 2001; 410 (6832): 1116-20, Izawa et al., Genes Dev. 2002; 16 (15): 2006-20, Itoh et al., Nat. Genet. 2010; 42 (7): 635-8). Thus, it is speculated that, in the process of flowering, there is a certain threshold for the expression level of a florigen gene, above which the flowering process starts. It is also known that a florigen gene is expressed specifically in a phloem of a vascular bundle in a leaf, and when the resulting florigen protein reaches a shoot apical meristem through the vascular bundle, the flower bud formation, that is, an elementary process of flowering, starts (Abe et al., Science 2005; 309 (5737): 1052-6, Tamaki et al., Science 2007; 316 (5827): 1033-6). On the other hand, there are also reports on examples where the amount of florigen when a flower bud is induced influences the form of inflorescences (Endo-Higashi and Izawa, Plant Cell Physiol. 2011), and where the existence of florigen in an amount more than necessary in rice causes very small inflorescences (Izawa et al., Genes Dev. 2002; (15): 2006-20). In addition, in the preliminary experiment by the present inventors using an ectopically-expressing OSH1 promoter (expressed in a shoot apical meristem, Sato et al., PNAS 1996; 93 (15): 8117-22), the flowering occurred earlier than a wild type line having no gene introduced therein, but a morphological abnormality was observed in the head. From the foregoing, there is a preferable range for the expression level of a florigen gene when the expression is induced.

As the promoter sensitive to the plant activator, it is preferable to use one capable of ensuring that the expression level when induced is at least 1/1000 or more (preferably 1/100 or more, further preferably 1/10 or more, most preferably equivalent or more) of that of ubiquitin, and also capable of suppressing the expression such that the expression level when not induced is at least 1/10 or less (preferably 1/100 or less, further preferably 1/1000 or less, most preferably 1/10000 or less) of that of ubiquitin. Moreover, the promoter used preferably increases the expression level when induced at least 5-fold or more (preferably 10-fold or more, further preferably 30-fold or more, most preferably 100-fold or more) as high as the expression level when not induced.

One form of the promoter which exhibits such expression characteristics and is suitably used in the present invention is a rice-derived promoter having a nucleotide sequence of SEQ ID NO: 1 (a promoter of a gene (12) described in the present Examples). This promoter has been selected as a promoter capable of ensuring preferable expression characteristics as a result of the microarray analysis of gene expressions in the field test using probenazole (Oryzemate) and isotianil (Routine) (see Example 3). In the present invention, it is possible to suitably use, other than the promoter of rice, promoters of orthologous genes in other plant species (for example, corn (Zea mays), sugarcane (Saccharum spp.), barley (Hordeum vulgare), wheat (Triticum spp.), sorghum (Sorghum bicolor)) to which the present invention is applied. Particularly, plants such as corn and sorghum have been confirmed to have orthologous genes of the gene (12) of rice. Promoters of these orthologous genes presumably have expression characteristics equivalent to those of the promoter of the (12) gene of rice. Further, it has been confirmed in Example 11 that the orthologous gene of the gene (12) in corn exhibits the inducible expression by a plant activator. From these facts, a promoter of the corn gene (SEQ ID NO: 133), which is orthologous to the gene (12) of rice, can also be suitably used in the present invention.

There is a report that an ScMYBAS1 promoter having a salicylic-acid inducible cis-sequence of sugarcane (Saccharum officinarum) and recognition sequence of a WRKY-type transcription factor involved in systemic acquired resistance (SAR) exhibits salicylic acid induction in a dicot plant (tobacco (Nicotiana tabacum)), which is evolutionally more distant from Poaceae monocot plants (Prabu and Prasad, Plant Cell Rep. 2012; 31 (4): 661-9). A plant activator is an inducer of systemic acquired resistance, and known to act on a signal transduction system via salicylic acid. From the foregoing, a salicylic-acid inducible promoter of monocot Poaceae plants functions also in dicot plants. Moreover, a SAR related gene induced by a plant activator is induced by a salicylic acid in rice and tobacco. From these, it seems that the compatibility of a promoter (or cis-sequence) capable of reacting with a plant activator is high among plant species; particularly, it is assumed that the compatibility is quite high between rice and other Poaceae plant species. From the foregoing, the present invention is suitably usable in a variety of Poaceae plants.

Further, once a promoter region of a certain gene is found out, those skilled in the art can identify shorter active fragments contained in the promoter region, or modify a nucleotide(s) of the promoter region to prepare a mutant DNA having the same activity, by utilizing general techniques. Meanwhile, a nucleotide sequence may also be mutated in nature.

Thus, as a DNA encoding the promoter used in the present invention, it is also possible to use a DNA having a nucleotide sequence of any one of SEQ ID NOs: 1, 133, and 137 in which one or more nucleotide s are substituted, deleted, added, and/or inserted, the DNA having an activity of the promoter sensitive to the plant activator. The number of nucleotides mutated is not particularly limited, as long as the resulting DNA has the activity of the promoter sensitive to the plant activator. Nevertheless, the number is generally 50 nucleotides or less, preferably 30 nucleotides or less, further preferably 10 nucleotides or less (for example, 5 nucleotides or less, 3 nucleotides or less, 2 nucleotides or less). An example of the methods well known to those skilled in the art for preparing a mutant DNA includes site-directed mutagenesis (Kramer, W. & Fritz, H. J. (1987) Oligonucleotide-directed construction of mutagenesis via gapped duplex DNA. Methods in Enzymology, 154: 350-367).

Further, by utilizing hybridization techniques (Southern, E. M., Journal of Molecular Biology, 98: 503, 1975), polymerase chain reaction (PCR) techniques (Saiki, R. K., et al. Science, 230: 1350-1354, 1985, Saiki, R. K. et al. Science, 239: 487-491, 1988), and the like, and utilizing information on the nucleotide sequence of any one of SEQ ID NOs: 1, 133, and 137, those skilled in the art can obtain DNAs (for example, promoters of orthologous genes) having a high homology with the DNA of SEQ ID NO: 1 and having the activity of the promoter sensitive to the plant activator, from the other rice cultivars, the other corn cultivars, or other plants (for example, Poaceae plants such as sugarcane, barley, wheat, and sorghum, or the like).

Thus, as the DNA encoding the promoter used in the present invention, it is also possible to use a DNA having a homology of 70% or more with the nucleotide sequence of any one of SEQ ID NOs: 1, 133, and 137, the DNA having the activity of the promoter sensitive to the plant activator. The homology is preferably 90% or more (for example, 95%, 96%, 97%, 98%, 99% or more). Note that such a homologous DNA in the present invention includes the above-described mutant DNA, as long as the homology therebetween is within the range (the same applies hereinafter).

In the present invention, the form of the “Hd3a gene” ligated downstream of the promoter sensitive to the plant activator is not particularly limited, and includes a cDNA, a genomic DNA, and a chemically synthesized DNA. These DNAs can be prepared by utilizing conventional means for those skilled in the art.

The “Hd3a gene” in the present invention typically includes a rice Kasalath cultivar DNA encoding a protein having an amino acid sequence of SEQ ID NO: 3 (for example, DNA having a nucleotide sequence of SEQ ID NO: 2), and a rice Nipponbare cultivar DNA encoding a protein having an amino acid sequence of SEQ ID NO: 5 (for example, DNA having a nucleotide sequence of SEQ ID NO: 4).

Moreover, when information on the nucleotide sequence of a particular Hd3a gene is obtained, those skilled in the art can modify the nucleotide sequence to obtain a DNA having the same activity of inducing flowering of a plant, although the amino acid sequence to be encoded is different from that of the original nucleotide sequence. Meanwhile, in nature also, the amino acid sequence of a protein to be encoded may undergo mutation by a mutation of the nucleotide sequence. Thus, the Hd3a gene of the present invention includes DNAs encoding a protein having an amino acid sequence (SEQ ID NO: 3 or 5) of an Hd3a protein of rice Kasalath or Nipponbare in which one or more amino acids are substituted, deleted, added, and/or inserted, the protein having an activity of inducing flowering of a plant. Herein, “more than one” refers to the number of amino acids modified, provided that the Hd3a protein after the modification still has the activity of inducing flowering of a plant. The number is generally 50 amino acids or less, preferably 30 amino acids or less, and further preferably 10 amino acids or less (for example, 5 amino acids or less, 3 amino acids or less, 2 amino acids).

Further, when a particular Hd3a gene is obtained, those skilled in the art can utilize information on the nucleotide sequence to obtain DNAs (for example, orthologous genes) encoding a homologous protein having the same activity of inducing flowering of a plant, from the other rice cultivars or other plants (for example, Poaceae plants such as corn, sugarcane, barley, wheat, and sorghum, or the like) by the above-described hybridization techniques and polymerase chain reaction (PCR) techniques. Thus, the Hd3a gene of the present invention also includes DNAs encoding a protein having a homology of 70% or more with the amino acid (SEQ ID NO: 3 or 5) of the Hd3a gene of rice Kasalath or Nipponbare, the protein having the activity of inducing flowering of a plant. The homology is preferably 90% or more (for example, 95%, 96%, 97%, 98%, 99% or more).

Whether or not the mutant DNA and the homologous DNA obtained as described above encode a protein having the activity of inducing flowering of a plant can be evaluated, for example, by examining whether or not introducing an expression construct, in which a test DNA is ligated downstream of the promoter having the nucleotide sequence of SEQ ID NO: 1, into a plant whose flowering has been suppressed by a constitutive expression of a Ghd7 gene to be described later allows the plant activator treatment to induce the flowering (in a case of rice, heading).

Note that, in order to isolate homologous DNAs of the promoter, the Hd3a gene, and the Ghd7 gene to be described later, generally, a hybridization reaction is carried out under stringent conditions. Examples of the stringent hybridization conditions include conditions of 6 M urea, 0.4% SDS, and 0.5×SSC; and stringent hybridization conditions equivalent thereto. When more stringent conditions, for example, conditions of 6 M urea, 0.4% SDS, and 0.1×SSC, are employed, isolation of a DNA having a higher homology can be expected.

Moreover, the sequence homology of the isolated DNA can be determined by utilizing a program of BLASTN (nucleic acid level) or BLASTX (amino acid level) (Altschul et al. J. Mol. Biol., 215:403-410, 1990). These programs are based on the algorithm BLAST by Karlin and Altschul (Proc. Natl. Acad. Sci. USA, 87:2264-2268, 1990, Proc. Natl. Acad. Sci. USA, 90:5873-5877, 1993). When a nucleotide sequence is analyzed by BLASTN, the parameter s are set to, for example, score=100, and wordlength=12. Meanwhile, when an amino acid sequence is analyzed by BLASTX, the parameters are set to, for example, score=50, and wordlength=3. Alternatively, when an amino acid sequence is analyzed by using the Gapped BLAST program, the analysis can be conducted as described in Altschul et al. (Nucleic Acids Res. 25: 3389-3402, 1997). When the BLAST and Gapped BLAST programs are used, the default parameters of each program are used. The specific procedures of these analysis methods are known. In addition, when sequences are compared, regions corresponding in both the sequences are compared with each other.

The Poaceae plant of the present invention also includes a plant further comprising an expression construct of a gene encoding a protein that suppresses an expression of an endogenous Hd3a gene but does not suppress an activity of an Hd3a protein. Generally, it is believed that all plants are genetically controlled such that the transition to the reproductive growth starts sooner or later for the procreation; otherwise, those that do not start the reproductive growth evolutionally go extinct. This nature means that when a flower bud induction is artificially controlled, a flower bud induction by an endogenous gene may serve as a factor disturbing the artificial control. In the plant in which the expression construct is introduced, the expression of the endogenous Hd3a gene is suppressed, but the expression of the exogenous Hd3a gene under the control of the heterologous promoter is not suppressed, and the activity of the expressed exogenous Hd3a protein is not suppressed, either. The present invention makes it possible to efficiently control the flowering time through gene expression switching using a combination of two types of switching: turning off the expression of the endogenous Hd3a gene by the expression construct for suppressing the flower bud formation; and turning on the expression of the exogenous Hd3a gene by the expression construct for inducing the flower bud formation.

The gene encoding the protein that suppresses the expression of the endogenous Hd3a gene but does not suppress the activity of the Hd3a protein is not particularly limited, but a Ghd7 gene can be suitably used. The flowering suppression function of Ghd7 that is activated under long-day conditions greatly contributes to the flowering time regulation of rice, a short-day plant. This flowering suppression function of Ghd7 is believed to be achieved by suppressing the expression of florigen genes Hd3a/RFT1 through suppression of the expression of a flowering promoter gene Ehd1 (rice- or monocot plant-specific flowering control gene, Japanese Unexamined Patent Application Publication No. 2003-339382, Doi et al., Genes Dev. 2004; 18 (8): 926-36) (Itoh et al., Nat. Genet. 2010; 42 (7): 635-8, Osugi et al., Plant Physiol. 2011). However, it has not been known that a Ghd7 protein does not suppress an activity of an Hd3a protein, but the present inventors have found out this fact for the first time.

The “Ghd7 gene” in the present invention is typically a DNA encoding a protein having an amino acid sequence of SEQ ID NO: 7 (for example, DNA having a nucleotide sequence of SEQ ID NO: 6). As the “Ghd7 gene” in the present invention, it is also possible to use a mutant DNA and a homologous DNA (for example, orthologous genes) as in the case of the Hd3a gene. To be more specific, the “Ghd7 gene” of the present invention also includes a DNA encoding protein having an amino acid sequence of SEQ ID NO: 7 in which one or more amino acids are substituted, deleted, added, and/or inserted, the protein having the same activities as above, and a DNA encoding a protein having a homology of 70% or more with the amino acid sequence of SEQ ID NO: 7, the protein having the same activities. As a matter of fact, it has recently been reported that there is a Ghd7 ortholog in corn, a Poaceae crop. The report states that the ortholog has a function to delay the flowering time as in the case of rice (Hung et al. (2012) Proc Natl Acad Sci USA 109: E1913-E1021).

Furthermore, in Poaceae plant sorghum, a gene having a similar sequence to that of Ghd7 has been found. It has been reported that, in the same Poaceae plants of wheat and barley also, homologous genes having a sequence similar to that of Ghd7 have a function to delay the flowering time (Trevaskis et al., Plant Physiol. 2006; 140 (4): 1397-405, Hemming et al., Plant Physiol. 2008; 147 (1): 355-66).

In order to effectively suppress the expression of the endogenous Hd3a gene, the gene is preferably ligated downstream of a constitutive expression promoter in the expression construct. For example, a corn-derived ubiquitin promoter is suitable as the constitutive expression promoter. The promoter has been known to function as a stronger constitutive expression promoter than a 35S promoter in rice (Cornejo et al., Plant Mol. Biol. 1993; 23 (3): 567-81).

The expression construct in the present invention may comprise a transcription termination factor in addition to the promoter and the gene. As the transcription termination factor, for example, a nos terminator or a 35S terminator can be used.

The Poaceae plant of the present invention can be produced, for example, by introducing the expression construct into a plant cell, and regenerating the transformed plant cell thus obtained. The “plant cell” into which the expression construct is introduced includes plant cells in various forms, for example, calli, suspended culture cells, protoplasts, leaf sections, and the like. The Poaceae plant from which the plant cell is derived is not particularly limited, and includes, besides rice, corn, sugarcane, barley, wheat, sorghum, and the like.

To introduce a vector into the plant cell, various methods known to those skilled in the art can be used, such as an Agrobacterium-mediated method, a polyethylene glycol method, an electroporation method (electroporation), and a particle gun method. A plant can be regenerated from the transformed plant cell by a method known to those skilled in the art in accordance with the type of the plant cell (see Toki et al. (1995) Plant Physiol. 100: 1503-1507).

For example, several techniques have been already established as the method for producing a transgenic rice plant, such as a method in which a gene is introduced using Agrobacterium and a plant is regenerated (Hiei et al. (1994) Plant J. 6: 271-282); a method in which a gene is introduced into protoplasts using polyethylene glycol and a plant (indica type rice cultivars are suitable) is regenerated (Datta, S. K. (1995) In Gene Transfer To Plants (Potrykus I and Spangenberg Eds.) pp. 66-74); a method in which a gene is introduced into protoplasts using electric pulse and a plant (japonica type rice cultivars are suitable) is regenerated (Toki et al. (1992) Plant Physiol. 100, 1503-1507); and a method in which a gene is directly introduced into cells by a particle gun method and a plant is regenerated (Christouet et al. (1991) Bio/technology, 9: 957-962). These are widely used in the technical field of the present invention. In the present invention, these methods can be suitably used.

Examples of the method for producing a transgenic corn plant includes the methods described in Ishida Y et al. (2007) Nat Protocols 2: 1614-1621, Hiei Y et al. (2006) Plant Cell Tiss Organ Cult 87: 233-243, and Ishida Y et al. (1996) Nat Biotechnol 14: 745-750.

Examples of the method for producing a transgenic sugarcane plant include the methods described in Arencibia, A. D. et al. (1998) Transgenic Research 7: 213-222; 1998, Bower, R. and Birch, R. G. (1992) Plant J 2: 409-416, Chen, W. H. et al. (1987) Plant Cell Rep. 6: 297-301, Elliott, A. R. et al. (1998) Aust J Plant Physiol 25: 739-743, Manickabasagam, M. et al. (2004) Plant Cell Rep 23: 134-143, and Zhangsun D. et al. (2007) Biologoa, Brarislava 62: 386-393.

As the method for producing a transgenic sorghum plant, suitably used are, for example, a method in which a gene is introduced into immature embryos or calli by an Agrobacterium method or a particle gun method and a plant is regenerated; and a method in which pollens having a gene introduced therein using ultrasound are used for pollination (J. A. Able et al., In Vitro Cell. Dev. Biol. 37: 341-348, 2001, A. M. Casas et al., Proc. Natl. Acad. Sci. USA 90: 11212-11216, 1993, V. Girijashankar et al., Plant Cell Rep 24: 513-522, 2005, J. M. JEOUNG et al., Hereditas 137: 20-28, 2002, V Girijashankar et al., Plant Cell Rep 24 (9): 513-522, 2005, Zuo-yu Zhao et al., Plant Molecular Biology 44: 789-798, 2000, S. Gurel et al., Plant Cell Rep 28 (3): 429-444, 2009, Z Y Zhao, Methods Mol Biol, 343: 233-244, 2006, A K Shrawat and H Lorz, Plant Biotechnol J, 4 (6): 575-603, 2006, D Syamala and P Devi Indian J Exp Biol, 41 (12): 1482-1486, 2003, Z Gao et al., Plant Biotechnol J, 3 (6): 591-599, 2005).

An example of the method for producing a transgenic wheat plant includes the method described in “Taiichi Ogawa, Japanese Journal of Pesticide Science, 2010, vol. 35, no. 2, pp. 160 to 164”.

Examples of the method for producing a transgenic barley plant include the methods described in Tingay et al. (Tingay S. et al. Plant J. 11: 1369-1376, 1997), Murray et al. (Murray F et al. Plant Cell Report 22: 397-402, 2004), and Travalla et al (Travalla S et al. Plant Cell Report 23: 780-789, 2005).

Once a transgenic plant having the expression cassette introduced in the genome is obtained, it is possible to obtain a progeny from the plant by sexual reproduction or asexual reproduction. Moreover, a propagation material (for example, a seed, a spike, a stub, a callus, a protoplast, and the like) is obtained from the plant or a progeny or a clone thereof, from which the plant can also be produced in mass. The present invention includes the Poaceae plant of the present invention, a progeny and a clone of the plant, and a propagation material of these.

From the foregoing, the present invention also provides a method for producing a Poaceae plant whose flowering time is controllable by a plant activator treatment, the method comprising the step of introducing into a Poaceae plant cell an expression construct in which an Hd3a gene is ligated downstream of a promoter sensitive to a plant activator, and regenerating the plant. Moreover, the present invention also provides the method further comprising a step of introducing an expression construct of a gene encoding a protein that suppresses an expression of an endogenous Hd3a gene but does not suppress an activity of an Hd3a protein. Further, the present invention also provides the method further comprising, after introducing into the Poaceae plant cell the expression construct in which the Hd3a gene is ligated downstream of the promoter sensitive to the plant activator, a step of selecting a cell in which one copy of the expression construct is inserted in a chromosome thereof. Note that the number of copies of the expression construct inserted in the chromosome can be checked by a genomic Southern blotting analysis, a PCR analysis, and the like as described later in Example 6. Specific embodiments of this method are as described above.

The Poaceae plant produced by the method of the present invention can be induced to flower at a certain timing by the plant activator treatment according to a method such as spraying. The present invention also provides a method for inducing flowering of a Poaceae plant, the method comprising the step of treating the Poaceae plant of the present invention with the plant activator. Specific embodiments of this method are as described above.

Furthermore, the present invention also provides an agent for inducing flowering of the Poaceae plant of the present invention, the agent comprising the plant activator as an active ingredient. Although specific embodiments of the plant activator are as described above, the agent of the present invention may further comprise, other than the plant activator, an adjuvant such as a carrier, an emulsifier, a dispersant, a spreader, a wetting agent, an adhesive, or a disintegrator, which are generally used in an agrochemical or the like.

Examples of the carrier include water; alcohols such as ethanol, methanol, isopropanol, butanol, ethylene glycol, and propylene glycol; ketones such as acetone, methyl ethyl ketone, and cyclohexanone; esters such as ethyl acetate; and solid carriers, for example, talc, bentonite, clay, kaolin, diatomaceous earth, white carbon, vermiculite, slaked lime, silica sand, ammonium sulfate, urea, and the like.

As the adjuvant other than the carrier, a surfactant is generally used, and examples thereof include anionic surfactants, cationic surfactants, nonionic surfactants, and amphoteric surfactants. These can be used alone or in a mixture of two or more.

The form of the agent of the present invention is not particularly limited, and examples thereof include emulsions, suspensions, powders, wettable powders, water-soluble powders, granules, pastes, aerosols, and the like.

The content of the plant activator in the agent of the present invention and the amount of the plant activator administered differ depending on the type of the plant, the type of the plant activator incorporated, the form, the use method, the use timing, and so forth. Those skilled in the art can prepare the plant activator as appropriate in accordance with these conditions.

### EXAMPLES

Hereinafter, the present invention will be more specifically described based on Examples. However, the present invention is not limited to the following Examples.

### Example 1

### Verification of Ability of Ghd7 Gene to Suppress Flower Bud Formation

Transgenic rice was produced in which a rice Ghd7 gene functioning to suppress flower bud formation was constitutively expressed by a corn-derived ubiquitin promoter. After the cultivation, the transgenic rice was examined for the flowering time (heading time).

A transformation plasmid used to produce Ghd7ox was prepared as follows. First, two oligo DNAs (BamHI-HA-F (5′-cgggatccatgtatccatacgatgttccagattatgctgtcggcgccggt tgg-3′/SEQ ID NO: 8) and StrepII-R (5′-ggcgcctcctttttcaaattgaggatgagaccaaccggcgccgacagcat a-3′/SEQ ID NO: 9)) were synthesized in such a manner that hemagglutinin (HA: YPYDVPDYA) and Strep-tagII (WSHPQFEK, IBA, http://www.iba-go.com) were linked in tandem, followed by annealing, and an HA-StrepII fragment in the form of double-stranded DNA was prepared using T4 DNA polymerase (Takara Bio Inc.). The underline indicates a BamHI sequence added. Moreover, an amino acid sequence VGAG was inserted as a linker sequence at a linking site of HA and Strep-tagII. The HA-StrepII fragment was treated with BamHI and phosphorylated with T4 Polynucleotide Kinase (Takara Bio Inc.). Thereby, a cloning fragment into a pENTR1A vector (Life Technologies Corporation) was prepared. Next, using as a template a cDNA synthesized from RNA collected at the time when Ghd7 was expressed in a rice cultivar Nipponbare, a cDNA containing a Ghd7 coding region was amplified by PCR using a primer Ghd7-F (5-ATGTCGATGGGACCAGCAGCCGGAG-3/SEQ ID NO: 10) and a primer Ghd7-EcoRI-R (5′-CGGAATTCTATCTGAACCATTGTCCAAGC-3′/SEQ ID NO: 11). The underline indicates an EcoRI sequence added. The Ghd7 cDNA was treated with EcoRI and phosphorylated with T4 Polynucleotide Kinase. The resulting DNA fragment and also the HA-StrepII fragment prepared as described above were cloned in BamHI and EcoRI sites of the pENTR1A at once, and sequenced. Thus, an entry vector pENTR1A/HA-StrepII-Ghd7 was prepared. An amino acid sequence GGA is inserted as a linker sequence between an HA-StrepII tag sequence and a Ghd7 start codon and within the same reading frame for these. Finally, the HA-StrepII-Ghd7 fragment was incorporated between a corn-derived ubiquitin promoter and a nos terminator of a destination vector pEASY-Ubipro (Matsui et al., Plant Cell Physiol 2010; 51 (10): 1731-1744) by an LR reaction (LR Clonase II, Invitrogen Corporation) to thus prepare a transformation binary plasmid pEASY/PUbi-HA-StrepII-Ghd7. This was used to transform a rice cultivar Kitaake (Ghd7 gene function-deficient cultivar). Thus, Ghd7ox was produced.

To transform the rice, an Agrobacterium-mediated gene introduction method was used. Concretely, known transformation methods for rice (Terada and Iida: Model Plant Laboratory Manual (edited by Masaki Iwabuchi, Kiyotaka Okada, and Ko Shimamoto, Springer-Verlag Tokyo) pp. 110-121, 2000, Hiei et al., Plant J. 1994; 6: 271-282, Toki et al., Plant Molecular Biology Reporter, 1997; 15 (1): 16-21) were utilized. As Agrobacterium, EHA105 was used, and the bacterial cells in which the above-described plasmid had been introduced by electroporation were used to infect calli. Moreover, in the chemical selection of the transformants, hygromycin was used at a final concentration of 50 mg/L.

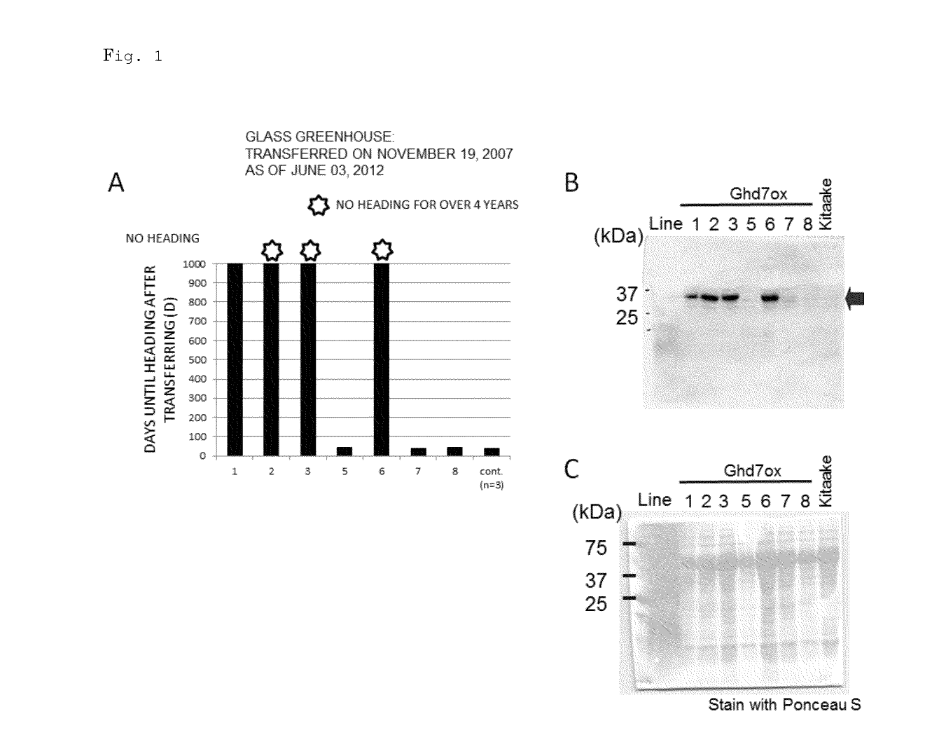

The transformation generation of Ghd7ox (T0 generation) was transferred to a glass greenhouse, and the heading time was examined. As a result, it was confirmed that lines strongly and constitutively expressing a Ghd7 protein did not flower for over 4 years (FIG. 1A). This result showed that as long as the Ghd7 gene strongly functioned, no flower bud was formed, so that no flowering occurred permanently.

Crude proteins were extracted from leaves of the Ghd7ox, and SDS-PAGE was carried out using a 10% acrylamide gel. In the crude protein extraction, 2×SDS Sample buffer (0.1 M Tris-HCl (pH 6.8), 4% SDS, 12% β-2 Mercaptoethanol, 50% Glycerol) was used. The electrophoresis was started at 20 mA cc and stopped when the electrophoresis dye BPB reached the lowest portion of the gel, and the resultant was transferred to a PVDF membrane (FLUOROTRANS W manufactured by Pall Corporation). The transferring to the membrane was performed at 15 mA/cm² for 60 minutes using TRANS-BLOT SD Semi-dry cell (Bio-Rad Laboratories, Inc.) as the transfer device. A solution obtained by adding ECL Advance Blocking Reagent (GE Healthcare) at a final concentration of 2% to a TBS-T (150 mM NaCl, 20 mM Tris-HCl (pH 7.5, 0.05% Tween 20) buffer was used as a blocking buffer. The blocking was performed over 1 hour, and then an antigen-antibody reaction was carried out. As a primary antibody, an anti-HA antibody (Anti-HA.11, Mouse-Mono (16B12), COVANS) was 5000-fold diluted and added to the blocking solution, and the antigen-antibody reaction was carried out at room temperature for 1 hour. Subsequently, the membrane was washed with a TBS-T solution for 15 minutes twice. Next, the antigen-antibody reaction was carried out with a secondary antibody. A 10000-fold diluted anti-IgG antibody (Anti-mouse IgG, peroxidase-linked species-specific whole antibody (from sheep), GE Healthcare) was added to the blocking solution, and the antigen-antibody reaction was carried out at room temperature for 1 hour. Thereafter, the membrane was washed with a TBS-T solution for 15 minutes twice. For the signal detection, ECL Plus Western Blotting Detection System (GE Healthcare) was used; an X-ray film (Hyperfilm ECL, GE Healthcare) for exposure; and Rendol (FUJIFILM Corporation) and Renfix (FUJIFILM Corporation) for development and fixation. FIG. 1B shows amounts of the Ghd7 protein accumulated in the Ghd7ox. Moreover, FIG. 1C shows the result of staining the membrane with a 1% Ponceau S solution after the above-described signal detection. It was verified that the analysis was conducted such that the amounts of the crude proteins were not significantly biased among the samples.

### Example 2

### Verification of Flowering by Expressing Exogenously-Introduced Hd3a Gene While Ghd7 Gene was Constitutively Expressed

Transgenic rices were produced in which a florigen gene Hd3a was expressed by several different promoters in a background where the flowering was strongly suppressed by the constitutive expression of Ghd7. The transgenic rices were examined for the flowering time (heading time).

A flowering-time control plasmid was prepared based on a binary vector pRiceFOX (Nakamura et al., Plant Mol. Biol. 2007; 65: 357-371). First, pRiceFOX was treated with HindIII and SalI. After each end was blunted, the vector was self-ligated. Thereby, modified pRiceFOX was prepared from which insert fragments cleaved with HindIII and SalI had been removed. Then, pHellsgate 8 (Helliwell and Waterhouse, Method 2003; 30 (4): 289-295) was treated with XhoI to cleave an AttR1-ccdB-AttR2 fragment. This fragment was inserted in an XhoI site of the modified pRiceFOX. Thereby, pRiceFOX/Gate corresponding to a Gateway system (Invitrogen Corporation) was prepared.

Next, an Hd3a cDNA of a rice Kasalath cultivar was amplified by PCR using a primer Hd3a-F-XbaI (5′-tctagaatggccggaagtgg-3′) and a primer Hd3a-R-KpnI (5′-ggtaccctagttgtagaccc-3′), cloned in pCR 8/GW/TOPO (Invitrogen Corporation), and sequenced. Moreover, in order to prepare an ADH5′UTR:Hd3a fragment containing a translational enhancer (ADH5′UTR (OsADH2 5′UTR), Sugio et al., J Biosci Bioeng. 2008; 105 (3): 300-2) inserted upstream of the Hd3a cDNA, PCR amplification was performed on an Hd3a cDNA fragment (a primer OsAdh_Hd3a_fw (5′-AAAAGAGGGGGATTAatggccggaagtggc-3′/SEQ ID NO: 12) and a primer Hd3a_kpn_rv (5′-GGAAATTCGAGCTCGGTACCctagttgtag-3′/SEQ ID NO: 13) were used for the PCR amplification) and an ADH5′UTR fragment (a primer OsAdh_enhancer_fw (5′-GAATTCCAAGCAACGAACTGCGAGTGA-3′/SEQ ID NO: 14) and a primer OsAdh_enhancer_rv (5′-TAATCCCCCTCTTTTTCAAAGAACAAG-3′/SEQ ID NO: 15) were used for the PCR amplification). Using an equimolar mixture of these as a template, PCR amplification was performed with a primer OsAdh_enhancer_fw2 (5′-CATAAGGGCCTCTAGAGAATTCCAAGCAAC-3′/SEQ ID NO: 16) and a primer Hd3a_kpn_rv. Then, the ADH5′UTR:Hd3a fragment ligated by PCR was cloned in pCR8/GW/TOPO and sequenced. The Hd3a cDNA fragment or the ADH5′UTR:Hd3a fragment was cleaved from these two types of plasmids by XbaI and KpnI treatments, and inserted in an XbaI site and a KpnI site located downstream of the AttR1-ccdB-AttR2 region of the pRiceFOX/Gate. Thus, pRiceFOX/Gate:Hd3a and pRiceFOX/Gate:ADH5′UTR:Hd3a were prepared. The underlines in the primer sequences indicate an XbaI sequence and a KpnI sequence added.

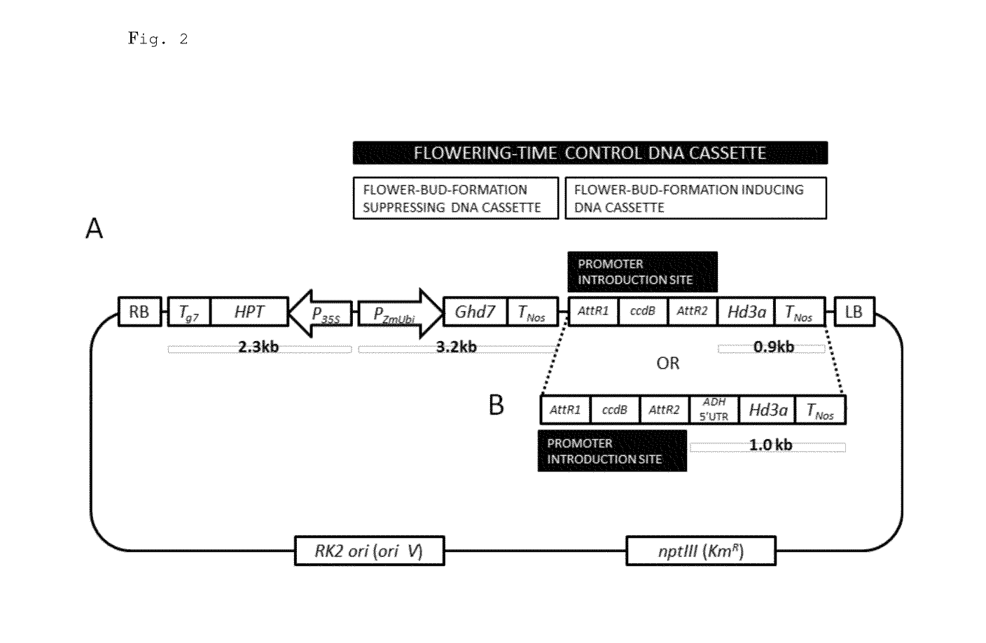

Further, using the pEASY/PUbi-HA-StrepII-Ghd7 described in Example 1 as a template, PCR amplification was performed with a primer Ubi-HindIII-F (5′-AAGCTTTGCAGCGTGACCCG-3′/SEQ ID NO: 17) and a primer NosT-HindIII-R (5′-AAGCTTgatctagtaacatag-3′/SEQ ID NO: 18). A PUbi-HA-StrepII-Ghd7 (PUbi:Ghd7) region involved in the constitutive expression of Ghd7 was sub-cloned in pCR 8/GW/TOPO. Then, this plasmid was treated with HindIII, and the resulting PUbi:Ghd7 fragment thus cleaved was inserted in a HindIII site of the pRiceFOX/Gate:Hd3a or the pRiceFOX/Gate:ADH5′UTR:Hd3a. Thus, a flowering-time control plasmid pRiceFOX/Ubi: Ghd7/Gate: Hd3a (FIG. 2A, SEQ ID NO: 19) and a flowering-time control plasmid pRiceFOX/Ubi:Ghd7/Gate:Adh5′UTR:Hd3a with the translational enhancer (FIG. 2B, SEQ ID NO: 20) were constructed.

A UBQ promoter, an Hd3a promoter, an OSH1 promoter, and a PLA1 promoter were each amplified by PCR and cloned in pCR 8/GW/TOPO (Invitrogen Corporation). Thereby, an entry vector for each promoter was constructed. Primers used to amplify each promoter region were as follows. For the UBQ promoter (2.0 kb): UbiF (5′-TGCAGCGTGACCCGGTCGTGC-3′/SEQ ID NO: 21) and UbiR (5′-AGTAACACCAAACAACAGG-3′/SEQ ID NO: 22). For the Hd3a promoter (2.0 kb): PHd3a-F1 (5′-aagaacatttacataataagcagg-3′/SEQ ID NO: 23) and PHd3a-R1 (5′-gggctgctggatcgagctgtgg-3′/SEQ ID NO: 24). For the OSH1 promoter (1.8 kb): OSH1-F (5′-ttctccaaccgtgcgtgtagg-3′/SEQ ID NO: 25) and OSH1-R (5′-gagagaagctcaagacacgca-3′/SEQ ID NO: 26). For the PLA1 promoter (2.0 kb): PLA1-F2 (5′-AAGCCACTTCCACGACAGGC-3′/SEQ ID NO: 27) and PLA1-R1 (5′-ggcggacacaaggtgtttgtgg-3′/SEQ ID NO: 28). Next, each promoter fragment was introduced into the promoter introduction site (AttR1-ccdB-AttR2) of the pRiceFOX/Ubi:Ghd7/Gate:Hd3a shown in FIG. 2 by an LR reaction (LR Clonase, Invitrogen Corporation). Thus, transformation plasmids were prepared. By the method described in Example 1, a rice cultivar Nipponbare was transformed with these plasmids obtained using the four different promoters.

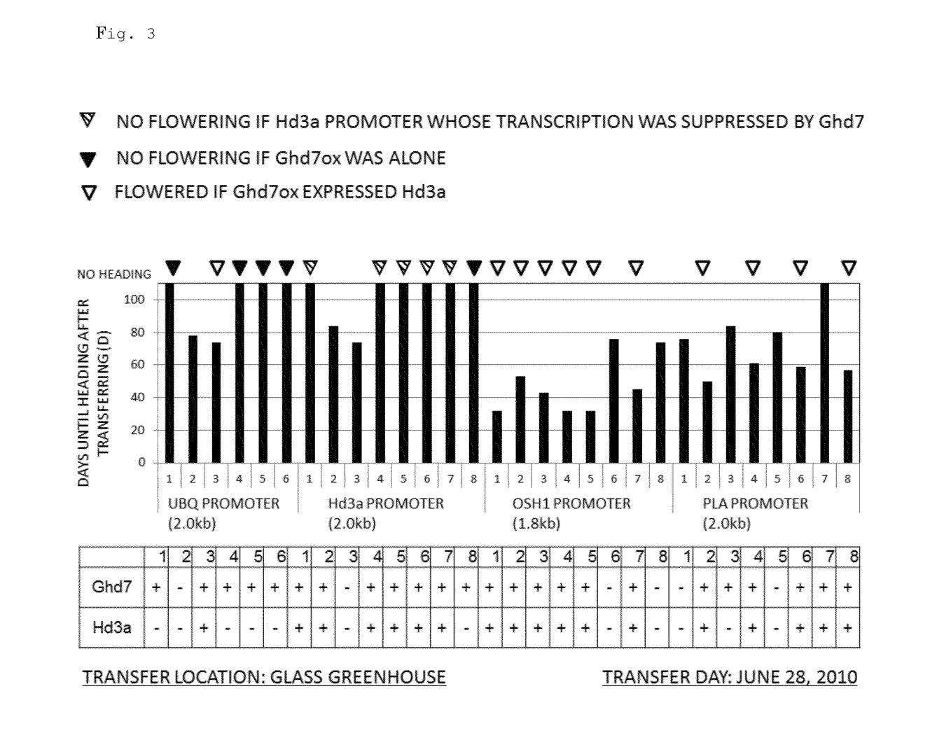

FIG. 3 shows the heading time of the rice transformants when exogenously introduced Hd3a was expressed by the different promoters in the genetic background where Ghd7 was constitutively expressed. The bar graph in FIG. 3 is a graph examining the heading time of the transgenic rice lines in the T0 generation transferred to a glass greenhouse. Moreover, the table at the bottom of FIG. 3 shows the presence or absence of the introduced genes in each line.

The presence or absence of the introduced genes was confirmed by a PCR analysis conducted using the genomic DNA from each line as a template. The genomic DNA was obtained as follows. First, leaves (approximately 1 cm) from the transformant were ground in a TPS buffer (100 mM Tris-Cl, 10 mM EDTA, 1 M KCl), and centrifuged (3000 rpm, 1 minute). The supernatant was subjected to isopropanol precipitation. Finally, the resultant was dissolved in a TE buffer, and the genomic DNA was extracted therefrom (simple extraction method). Primers used in the PCR analysis were as follows. For Ghd7: 3UBQMF2 (5′-tttagccctgccttcatacgct-3′/SEQ ID NO: 29) and 3Lhd4R1 (5′-CGTCGTTGCCGAAGAACTGG-3′/SEQ ID NO: 30). For Hd3a: Hd3a/F (Xba) (5′-tctagaatggccggaagtgg-3′/SEQ ID NO: 31) and Hd3a/Rsac (5′-gagctcctagttgtagaccc-3′/SEQ ID NO: 32). For Hpt: P35S1 (5′-TCCACTGACGTAAGGGATGA-3′/SEQ ID NO: 33) and Nos3 (5′-ATCAGCTCATCGAGAGCCT-3′/SEQ ID NO: 34).

As a result of cultivating the transgenic rices in which the Hd3a gene was expressed by the UBQ promoter, the OSH1 promoter, or the PLA1 promoter, the flowering was observed in the lines having exogenously introduced Hd3a linked to the corresponding promoter, although Ghd7 was constitutively expressed. On the other hand, in the case of using the Hd3a promoter whose transcription was down-regulated by Ghd7, no flowering occurred. These results showed that even in the case where Ghd7 was constitutively expressed at a high level, it was possible to cause flowering by expressing exogenously introduced Hd3a. Additionally, these results showed that the function of Ghd7 to suppress flower bud formation was exhibited through downregulation of Hd3a at a transcript ion level but did not inhibit the function of an Hd3a protein to promote flower bud formation.

This Example demonstrated that regarding a transformant whose flowering was suppressed as a result of constitutively expressing exogenous Ghd7 at a high level, it was also possible to control the flowering time by expressing Hd3a using an inducible promoter which was not regulated at a transcription level by Ghd7.

### Example 3

### Transcriptome Analysis on Plant Activators Sprayed in Field

As chemicals for artificially controlling flower bud formation through gene expression, two different plant activators, probenazole (Oryzemate 1 kg granule (containing 24% probenazole, Meiji Seika Kaisha, Limited)) and isotianil (Routine 1 kg granule (containing 3% isotianil, Bayer CropScience AG)), were used to conduct a spraying test on rice Nipponbare planted in a field. The changes in the transcriptome of leaf blade samples collected over time were analyzed using microarrays.

The spray treatment with the plant activators (Oryzemate granule: 1 kg/a, Routine granule: 1 kg/a) was performed in the field where a control plot (untreated plot with the chemicals) and treated plots with the chemicals (two treated plots where Oryzemate was sprayed and where Routine was sprayed) were provided in such a way that one plot was not contaminated with water from the other plots. After the chemicals were sprayed (started on 2010 Jul. 5), leaf blades were collected from each treated plot on Day 1 (2010 Jul. 6), Day 3 (2010 Jul. 8), Day 7 (2010 Jul. 12), Day 14 (2010 Jul. 19), and Day 30 (2010 Aug. 4), and frozen with liquid nitrogen. Then, total RNA was extracted us ing RNeasy Plant Mini Kit (Qiagen N.V.). To quantify the total RNA, Nanodrop (Thermo Fisher Scientific Inc.) was used. The total RNA was labeled by a two-color method (Cy3 or Cy5) according to the protocol recommended by the manufacturer, and 800 ng of labeled cRNA probe was used for hybridization to one microarray.

The microarray used was a rice oligo DNA microarray (rice 44K custom array, Agilent Technologies, Inc.). This microarray was designed based on the Rice Annotation Project (RAP, http://rapdb.dna.affrc.go.jp), and provided with 44000 DNA probes. Meanwhile, multiple probes hybridize to one gene such that the probes overlap with one another in some cases. Hence, these probes corresponded to 27201 genes as a whole. In a case where multiple probes were present for one gene, an average value of the signal intensities of the probes was set as a signal intensity of the corresponding gene. The microarray data were processed by the gspline method implemented in R and Bioconductor package (http://www.r-project.org/; Workman et al., Genome Biol. 2002; 3 (9): research0048), and the data obtained after the normalization were analyzed using Excel.

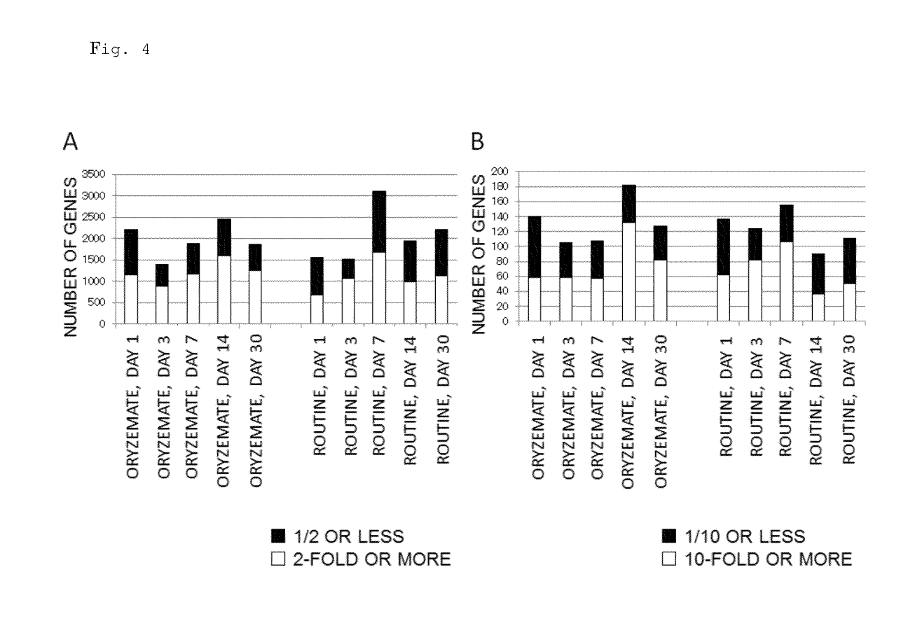

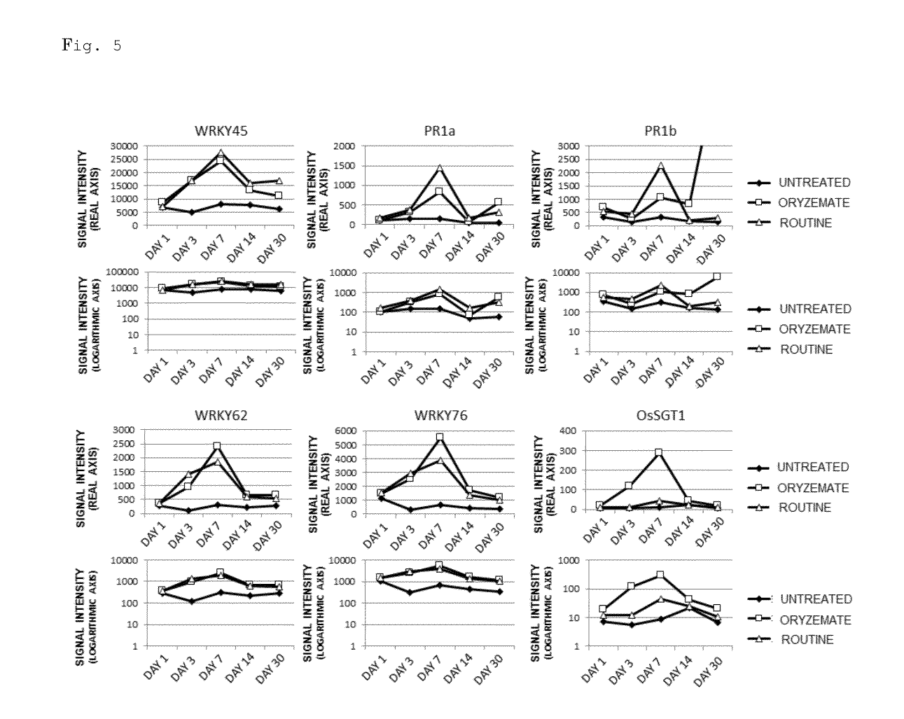

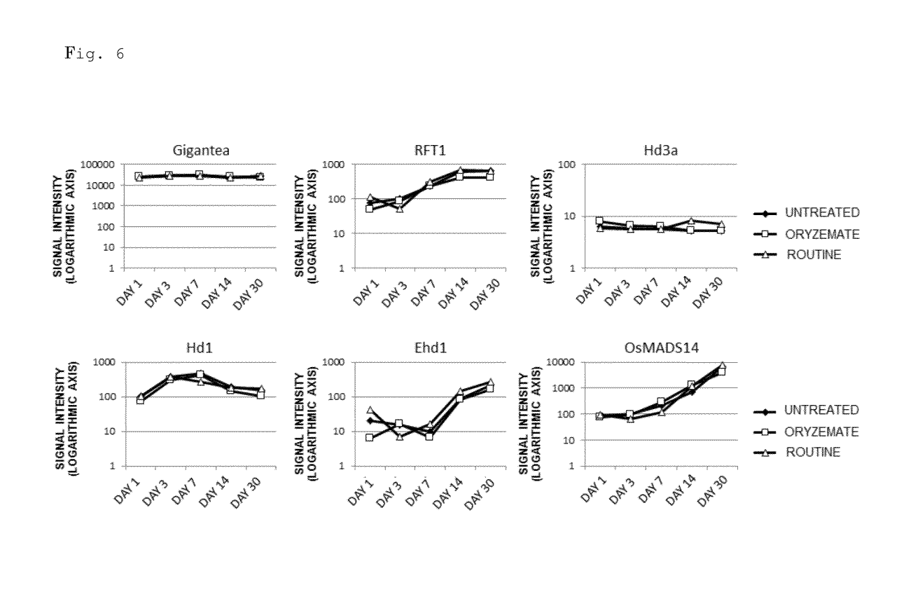

The changes over time in the transcriptome of leaf blade samples collected in the field were analyzed, and the expression level was compared between the samples in the treated plots and untreated plot with the chemicals at each collection timing. As a result, by each of the two chemicals, increases and decreases (2-fold or more and ½ or less) at the expression level were observed from approximately 2000 genes (FIG. 4a). Moreover, several tens or more of genes whose expression level was increased 10-fold or more were also found (FIG. 4b). Further, examining the microarray data on SAR related genes such as WRKY45 and genes having been reported to be induced by plant activators such as probenazole and BTH (Shimono et al., Plant Cell 2007; 19: 2064-2076, Umemura et al., Plant J. 2009; 57: 463-472) confirmed that the expressions were induced by Oryzemate granule and Routine granule (FIG. 5). On the other hand, when the expressions of flowering-time control related genes (Izawa, J Exp Bot. 2007; 58 (12): 3091-7, Izawa, Plant Cell Environ. 2012; 35 (10): 1729-41) were examined, no clear change in the expression by the plant activators was observed (FIG. 6). These results showed that gene expressions were sufficiently induced by a plant activator treatment in a field, and showed that a plant activator was applicable as a chemical for inducing an Hd3a expression in order to promote flower bud formation.

Next, the gene group observed to be induced by the plant activators in the result of the transcriptome analysis on the field test was searched for optimal inducible promoters for inducting the expression of exogenously introduced Hd3a. First, in each of the microarray data obtained by using the two chemicals, the fold change of the signal intensity in the treated plot relative to the signal intensity in the untreated plot at a data point on Day 1, Day 3, Day 7, or Day 14 after the chemical treatment was determined for each of the 27201 genes provided to the rice 44K oligo DNA microarray. The fold change values at the four data points were used to calculate Rank product (Breitling et al., FEBS Lett. 2004; 573 (1-3): 83-92) values. The smallest 60 values or less thereamong were set as an inducible gene group in the search range.

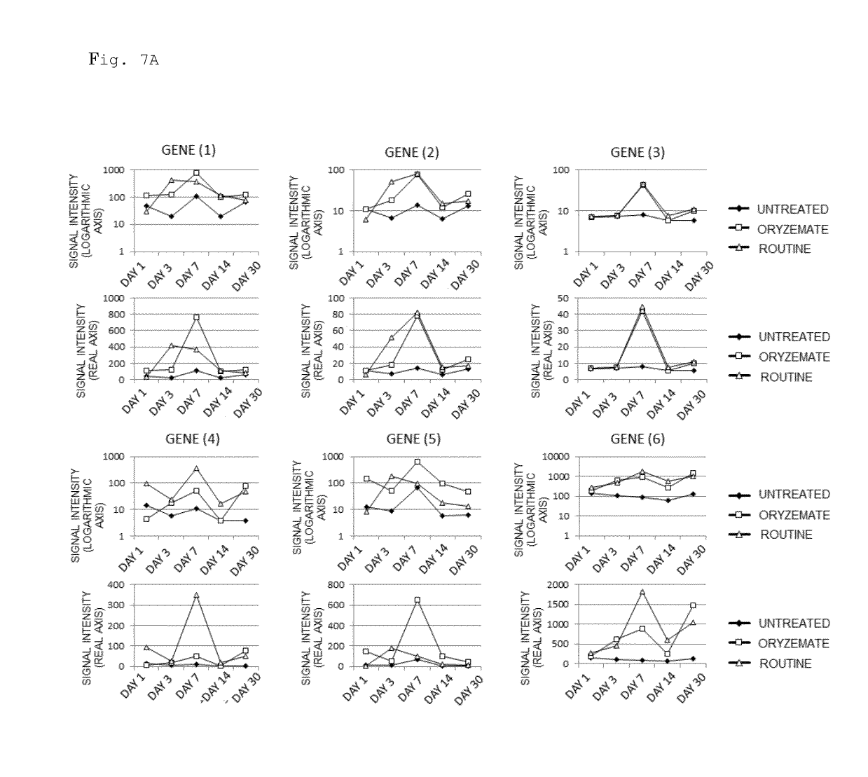

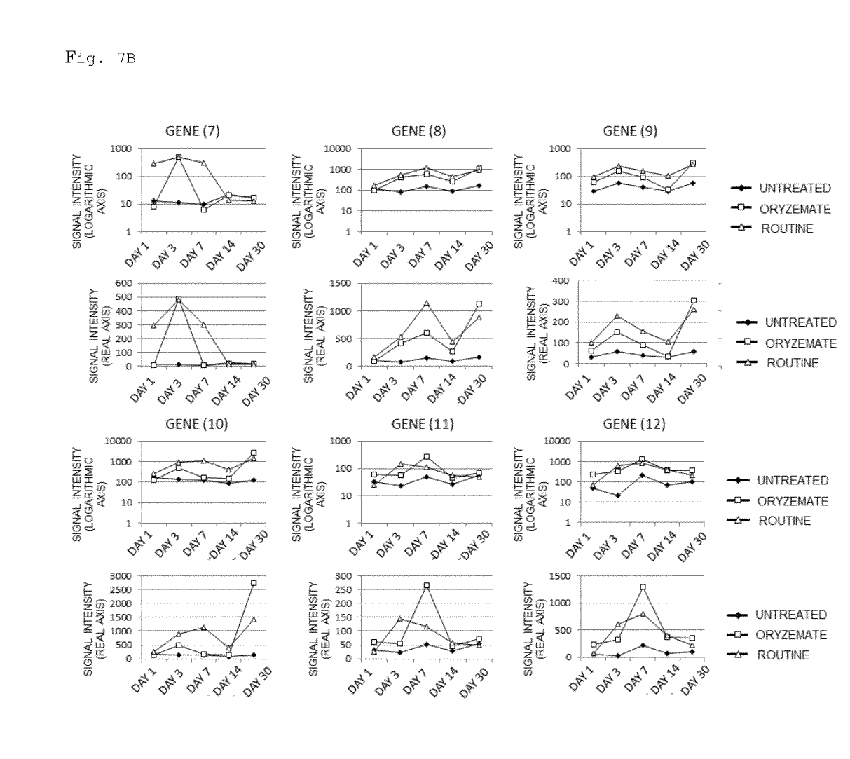

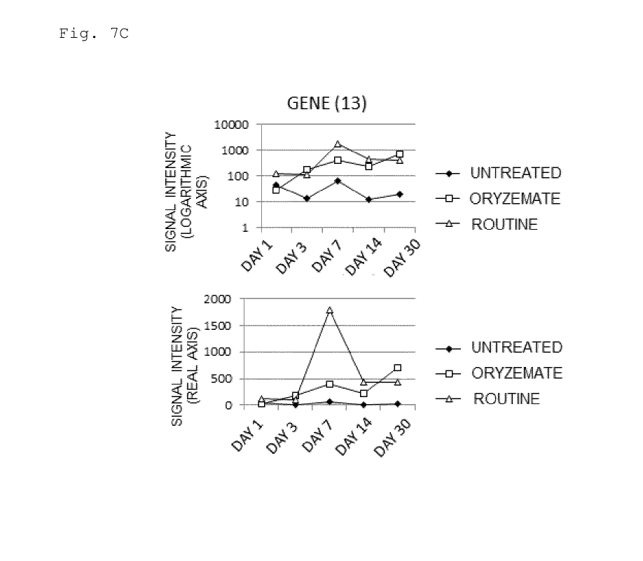

As the intrinsic expression of the Hd3a gene, 1) the gene is strongly expressed when conditions such as day length are satisfied; however, during the vegetative growth, the expression is suppressed or the gene is generally hardly expressed, and 2) the gene is specifically expressed in a phloem of a vascular bundle in a leaf but not expressed in the other organs and tissues such as root and stem. In consideration of these conditions, first, 38 genes whose expression level when not induced was low (i.e., signal intensity of 250 or less) were selected in the case of using Oryzemate, and 41 genes were selected in the case of using Routine, from the above-described gene group in the search range. Then, among the selected genes, ones that were expressed in a leaf but not expressed or expressed at a low level in the other organs and tissues (excluding ones expressed in a reproductive organ) were sorted by utilizing RiceXpro (rice transcriptome database, Sato et al., Nucleic Acids Research 2011; 39: D1141-1148). Among the genes, ten genes were found from the data obtained by using Oryzemate, and 13 genes were found from the data obtained by using Routine. Promoters of ten genes in total from these were selected as target inducible promoter candidates (Table 1). Furthermore, three genes were selected from the microarray data on leaves treated with BTH known as a plant activator (Shimono et al., Plant Cell. 2007; 19 (6): 2064-76), the genes not overlapping with the genes selected from the data obtained by using Oryzemate and Routine. Thus, promoters of 13 such genes in total were selected as inducible promoter candidates (Tables 1 and 2, FIG. 7). As to the genes selected in both of the cases of using Oryzemate and Routine, ones having the higher rank in the Rank product values therebetween were selected. Note that the nucleotide sequences of the promoters of the genes (1) to (11) and (13) are shown in SEQ ID NOs: 35 to 46. The nucleotide sequence of the promoter of the gene (12) is shown in SEQ ID NO: 1 as described above.

TABLE 1
Candidate gene promoters

Candidate genes selected from the transcriptome analysis on the Oryzemate spraying test

| | Day 1 | Day 3 | Day 7 | Day 14 | Oryzemate | Oryzemate |
|---|---|---|---|---|---|---|
| | Oryzemate | Oryzemate | Oryzemate | Oryzemate | Rank | Rank of |
| | treatment/ | treatment/ | treatment/ | treatment/ | product | rank products |
| Candidate | untreatment | untreatment | untreatment | untreatment | (Days 1, 3, | (Days 1, 3, |
| gene | (Rank) | (Rank) | (Rank) | (Rank) | 7, 14) | 7, 14) |
| gene (5) | 12.0 (44) | 5.8 (123) | 9.9 (60) | 16.8 (58) | 65.9 | 6 |
| gene (12) | 4.7 (279) | 15.2 (30) | 6.0 (130) | 5.1 (326) | 122.7 | 14 |
| gene (1) | 2.5 (692) | 6.1 (115) | 7.1 (88) | 5.2 (320) | 217.6 | 38 |

Candidate genes selected from the transcriptome analysis on the Oryzemate spraying test

| | Day 1 | Day 3 | Day 7 | Day 14 | Routine | Routine |
|---|---|---|---|---|---|---|
| | Routine | Routine | Routine | Routine | Rank | Rank of |
| | treatment/ | treatment/ | treatment/ | treatment/ | product | rank products |
| Candidate | untreatment | untreatment | untreatment | untreatment | (Days 1, 3, | (Days 1, 3, |
| gene | (Rank) | (Rank) | (Rank) | (Rank) | 7, 14) | 7, 14) |
| gene (5) | 0.7 | 20.9 (23) | 1.5 (3041) | 3.1 (322) | 869.2 | 305 |
| gene (12) | 1.4 (2127) | 28.7 (8) | 3.7 (555) | 5.5 (103) | 176.6 | 23 |
| gene (1) | 0.6 | 21.4 (19) | 3.4 (644) | 5.8 (94) | 413.9 | 87 |

Candidate genes selected from the transcriptome analysis on the Routine spraying test

| | Day 1 | Day 3 | Day 7 | Day 14 | Routine | Routine |
|---|---|---|---|---|---|---|
| | Routine | Routine | Routine | Routine | Rank | Rank of |
| | treatment/ | treatment/ | treatment/ | treatment/ | product | rank products |
| Candidate | untreatment | untreatment | untreatment | untreatment | (Days 1, 3, | (Days 1, 3, |
| gene | (Rank) | (Rank) | (Rank) | (Rank) | 7, 14) | 7, 14) |
| gene (13) | 2.8 (333) | 7.8 (117) | 28.2 (23) | 34.3 (9) | 53.3 | 1 |
| gene (7) | 22.2 (19) | 43.1 (4) | 30.5 (18) | 0.6 | 76.9 | 2 |
| gene (4) | 6.5 (93) | 4.2 (267) | 32.3 (12) | 4.3 (162) | 83.4 | 3 |
| gene (6) | 1.9 (735) | 4.4 (250) | 20.4 (30) | 9.9 (39) | 121.1 | 8 |
| gene (10) | 1.5 (1527) | 6.4 (158) | 8.9 (130) | 4.6 (148) | 261 | 36 |
| gene (8) | 1.5 (1561) | 6.8 (140) | 7.5 (179) | 4.8 (132) | 268.1 | 42 |
| gene (9) | 3.4 (220) | 3.9 (298) | 3.8 (529) | 3.6 (233) | 299.8 | 53 |

Candidate genes selected from the transcriptome analysis on the Routine spraying test

| | Day 1 | Day 3 | Day 7 | Day 14 | Oryzemate | Oryzemate |
|---|---|---|---|---|---|---|
| | Oryzemate | Oryzemate | Oryzemate | Oryzemate | Rank | Rank of |
| | treatment/ | treatment/ | treatment/ | treatment/ | product | rank products |
| Candidate | untreatment | untreatment | untreatment | untreatment | (Days 1, 3, | (Days 1, 3, |
| gene | (Rank) | (Rank) | (Rank) | (Rank) | 7, 14) | 7, 14) |
| gene (13) | 0.6 (25453) | 13.3 (40) | 6.3 (118) | 18.0 (53) | 282.5 | 50 |
| gene (7) | 0.6 | 43.9 (2) | 0.6 | 0.9 | 2166.4 | 1263 |
| gene (4) | 0.3 | 3.1 (353) | 4.6 (221) | 1.0 | 2194.6 | 1285 |
| gene (6) | 1.4 (3177) | 5.9 (121) | 9.9 (58) | 4.3 (421) | 311.3 | 55 |
| gene (10) | 0.8 | 3.5 (274) | 1.4 (3488) | 1.7 (2260) | 2663.2 | 1643 |
| gene (8) | 0.9 | 5.3 (144) | 4.0 (276) | 2.8 (828) | 903.3 | 345 |
| gene (9) | 2.1 (1060) | 2.6 (478) | 2.2 (925) | 1.2 (5942) | 1291.8 | 590 |

Candidate genes selected from the microarray data on the BTH spraying test
(Shimono et al., Plant Cell. 2007 June; 19 (6): 2064-76

| | Day 1 | Day 3 | Day 7 | Day 14 | Oryzemate | Oryzemate |
|---|---|---|---|---|---|---|
| | Oryzemate | Oryzemate | Oryzemate | Oryzemate | Rank | Rank of |
| | treatment/ | treatment/ | treatment/ | treatment/ | product | rank products |
| Candidate | untreatment | untreatment | untreatment | untreatment | (Days 1, 3, | (Days 1, 3, |
| gene | (Rank) | (Rank) | (Rank) | (Rank) | 7, 14) | 7, 14) |
| gene (2) | 1.0 | 2.7 (444) | 5.7 (151) | 1.9 (1738) | 1133.7 | 481 |
| gene (3) | 1.0 | 1.1 (6691) | 5.3 (175) | 1.0 | 3568.1 | 2374 |
| gene (11) | 1.8 (1384) | 2.4 (557) | 5.2 (178) | 1.7 (2324) | 751.5 | 273 |

Candidate genes selected from the microarray data on the BTH spraying test
(Shimono et al., Plant Cell. 2007 June; 19 (6): 2064-76

| | Day 1 | Day 3 | Day 7 | Day 14 | Routine | Routine |
|---|---|---|---|---|---|---|
| | Routine | Routine | Routine | Routine | Rank | Rank of |
| | treatment/ | treatment/ | treatment/ | treatment/ | product | rank products |
| Candidate | untreatment | untreatment | untreatment | untreatment | (Days 1, 3, | (Days 1, 3, |
| gene | (Rank) | (Rank) | (Rank) | (Rank) | 7, 14) | 7, 14) |
| gene (2) | 0.5 | 7.7 (118) | 5.9 (268) | 2.4 (582) | 833.3 | 289 |
| gene (3) | 1.1 (8607) | 1.1 (8301) | 5.6 (291) | 1.3 (3454) | 2887.1 | 1479 |
| gene (11) | 0.8 (24107) | 6.4 (162) | 2.3 (1324) | 2.1 (827) | 1438 | 607 |

TABLE 2
Candidate genes

| | Locus TD | |
|---|---|---|
| Gene | (RAP DB) | Description |
| gene (1) | Os01g0567200 | Conserved hypothetical protein. |
| gene (2) | Os03g0629800 | Conserved hypothetical protein. |
| gene (3) | Os04g0556400 | Similar to Cis-zeatin O-glucosyltraneferase 1 (EC 2.4.1.215) (cisZOG1). |
| gene (4) | Os07g0687400 | VQ domain containing protein. |
| gene (5) | Os12g0458100 | Transferase family protein. |
| gene (6) | Os04g0339000 | Cytochrome P450 family protein. |
| gene (7) | Os06g0371600 | Leucine-rich repeat, cysteine-containing containing protein. |
| gene (8) | Os06g0671300 | Cytochrome P450 family protein. |
| gene (9) | Os07g0489000 | Plant lipid transfer protein/Par allergen family protein. |
| gene (10) | Os11g0514400 | Similar to Somatic embryogenesis receptor kinase 1. |
| gene (11) | Os01g0108500 | Conserved hypothetical protein. |
| gene (12) | Os01g0108400 | Basic helix-loop-helix dimerisation region bHLH domain containing protein. |
| gene (13) | Os08g0428200 | Similar to Typical P-type R2R3 Myb protein (Fragment). |

### Example 4

### Production of Flowering-Induced Lines and Verification of Flowering Induction

Putative promoter regions of the selected 13 genes were each amplified by PCR using primers shown in Table 3 and cloned in pCR 8/GW/TOPO (Invitrogen Corporation). Thereby, an entry vector for each gene promoter was constructed.

TABLE 3
Primer sequences used to isolate gene promoters

| Gene | Primer name | Sequence (5′-3′) |
|---|---|---|
| gene (1) | Os01g0567200_fw2 | GCATTCACTCTCCCGTTCTTGATCGCTT/SEQ ID NO: 47 |
| | Os01g0567200_rv2 | CCGGCAAAAGACCAACTAGGGACAAACC/SEQ ID NO: 48 |
| gene (2) | Os03g0629800_fw2 | attgccgatccatctacatgagtcaa/SEQ ID NO: 49 |
| | Os03g0629800_rv2 | GGTCTTTGGATCTCGCACCTCCACCGC/SEQ ID NO: 50 |
| gene (3) | Os04g0556400_fw | TTATGTCAGCAATATAAGCATTTCTGA/SEQ ID NO: 51 |
| | Os04g0556400_rv | AGGCTCGATGACTGTGCTCAACC/SEQ ID NO: 52 |
| gene (3) 3′UTR | Os04g0556400_3′UTR_fw | GGTACCCTGATTCTTGCCTGGCCCATG/SEQ ID NO: 53 |
| | Os04g0556400_3′UTR_rv | GGTACCGGTCCACAAATGATGTCCAATTC/SEQ ID NO: 54 |
| gene (4) | Os07g0687400_fw2 | AATGAGTAGCACGAGGACTCACCCCTG/SEQ ID NO: 55 |
| | Os07g0687400_rv2 | TCTAGAGCTTTTTGTGAGCGTGGTGTG/SEQ ID NO: 56 |
| gene (5) | Os12g0458100_fw3 | ccaatatccacaagaaacagaggacaa/SEQ ID NO: 57 |
| | Os12g0458100_rv3 | GATTCTACGTACGTTTGTATGGATGGA/SEQ ID NO: 58 |
| gene (6) | Os04g0339000_fw | caaatttcatgtggatggtcctgatcac/SEQ ID NO: 59 |
| | Os04g0339000_rv | GTCCGTACGATGTGCTGTACGCACTAG/SEQ ID NO: 60 |
| gene (7) | Os04g0371600_fw | ACAGTATACACTGACttaggtggtgtt/SEQ ID NO: 61 |
| | Os04g0371600_rv | CAATGTTGTAGAGCTGCTTGACACAAG/SEQ ID NO: 62 |
| gene (8) | Os06g0671300_fw | GTCACCACTAGGTAGATCGATCATCCCT/SEQ ID NO: 63 |
| | Os06g0671300_rv | GATGGCGCGCAGCGTCAGGTCGGTAAG/SEQ ID NO: 64 |
| gene (9) | Os07g0489000_fw | TATAGCTTGTGTTGCGCACCTCGAAAG/SEQ ID NO: 65 |
| | Os07g0489000_rv | CGTGTGAGATGGATGGAGATCGTATGAC/SEQ ID NO: 66 |
| gene (10) | Os11g0514400_fw | TCATTCACCATGTGCTATGGAGACAAC/SEQ ID NO: 67 |
| | Os11g0514400_rv | TGCTGCAAGACCTGAGTAGTTCTTGG/SEQ ID NO: 68 |
| gene (11) | Os01g0108500_fw | AGGATTTTGTGATGGGCTTGGCCCAAC/SEQ ID NO: 69 |
| | Os01g0108500_rv | GCTCGTCGGCAAAAGACCAATTAGGGA/SEQ ID NO: 70 |
| gene (12) | Os01g0108400_fw | TACAAAGGAGTCCACATCAACCCTCCAG/SEQ ID NO: 71 |
| | Os01g0108400_rv | GACGATGGCTAACTGGTCGTCTCAGCC/SEQ ID NO: 72 |
| gene (13) | Os08g0428200_fw | ACTCTGAATAACACTGAAACATTCCATTG/SEQ ID NO: 73 |
| | Os08g0428200_rv2 | TGATGATCTCCTACCTTAAGCTGCTGA/SEQ ID NO: 74 |

Then, each gene promoter was incorporated into the promoter introduction site of the flower-bud-formation inducing DNA cassette in the flowering-time control plasmid (pRiceFOX/Ubi:Ghd7/Gate:Hd3a, FIG. 2a) by the LR reaction. Thus, transformation binary plasmids were prepared. As to the gene (3), other than the promoter region, a 3′UTR region (SEQ ID NO: 75) was inserted in a kpnI site located immediately downstream of the Hd3a cDNA to prepare a transformation binary plasmid. These were each used to transform a rice cultivar Nipponbare. The transformation was carried out by the method described in Example 1. As to the genes (6) and (8), since these are paralogous genes (overlapping genes), no transformant was produced using the promoter of the gene (8).

Two replicate individuals were prepared by dividing tillers of each line of the transformants produced using the gene promoters. One of the individuals was transferred to a treated plot with a plant activator, while the other individual was transferred to an untreated plot with the plant activator. Then, a chemical spraying test was conducted. Concretely, the individuals for the treatment/untreatment were planted in different pots with soil, each immersed in a flooded container for the treatment/untreatment together with the pot, grown under the flooded condition, and subjected to the chemical spraying (the chemical was sprayed to the pot).

When the tillers were transferred, the genomic PCR analysis was conducted. The lines confirmed to have the introduced gene without missing were thus transferred. The genomic DNA was extracted according to the simple method described in Example 2. Primers used in the PCR analysis were as follows. For Ghd7: 3UBQMF2 (5′-tttagccctgccttcatacgct-3′/SEQ ID NO: 76) and 3Lhd4R1 (5′-CGTCGTTGCCGAAGAACTGG-3′/SEQ ID NO: 77). For Hd3a: Hd3a/F (XbaI)) (5′-tctagaatggccggaagtgg-3′/SEQ ID NO: 78) and Hd3a/R (sacI) (5′-gagctcctagttgtagaccc-3′/SEQ ID NO: 79). For Hpt: P35S1 (5′-TCCACTGACGTAAGGGATGA-3′/SEQ ID NO: 80) and Nos3 (5′-ATCAGCTCATCGAGAGCCT-3′/SEQ ID NO: 81). To confirm the introduced gene Hd3a in the transformants obtained by using the promoters of the genes (3) and (12), gene (3)_colony_fw (5′-ttgtggatgcccTAACAGCTTGG-3′/SEQ ID NO: 82) and gene (12)_seqfw16 (5′-GCTATTAGCTTGCTTTGG-3′/SEQ ID NO: 83) were used as forward primers in place of Hd3a/F (XbaI).

The chemical spray treatment was performed after the divided plant was grown for 2 weeks to 4 weeks in a glass greenhouse or a growth chamber (long-day conditions: 14.5 hours of the light period: 9.5 hours of the dark period, temperature setting: 28° C. during the light period: 25° C. during the dark period, illumination: a metal-halide lamp of 500 μE). Regarding the transformants obtained by using the promoters of the genes (1) and (11), the flag leaf/heading was observed at the transfer stage or the chemical spray treatment stage, and hence the subsequent analysis was not conducted. The transformants obtained by using the promoters of the genes (2) to (7), (9), and (10) were chemically treated with Routine 1 kg granule or Oryzemate 1 kg granule in an amount of 1.0 g/individual. Moreover, the transformants obtained by using the promoters of the genes (12) and (13) were chemically treated with Routine 1 kg granule or Oryzemate 1 kg granule in an amount of 0.5 g/individual every 5 days three times in total. After the chemical treatment, leaf blades were collected from the plants of each line in the untreated plot and the treated plot, and the induction of the gene expression by the chemicals was examined by a quantitative RT-PCR analysis.

To extract total RNA from the collected leaf blades, TRIZOL Reagent (Invitrogen Corporation) was used. Moreover, to synthesize the cDNA from 2 μg of the total RNA, Oligo d(T)₁₂₋₁₈ primer (Invitrogen Corporation) was used for the synthesis with SuperscriptII Reverse Transcriptase (Invitrogen Corporation) according to the manual. In the real-time PCR, ABI 7900 Real-Time PCR System (Applied Biosystems Inc.) was used, and the quantitative RT-PCR analysis was conducted by the SYBR Green method (as the reagent, Power SYBR Green PCR Master Mix (Applied Biosystems Inc.) was used) and the Taq Man probe method (as the reagent, qPCR Mastermix (Eurogentec) was used). Table 3 shows the sequences of the primers used in the quantitative RT-PCR analysis and the Taq Man probes.

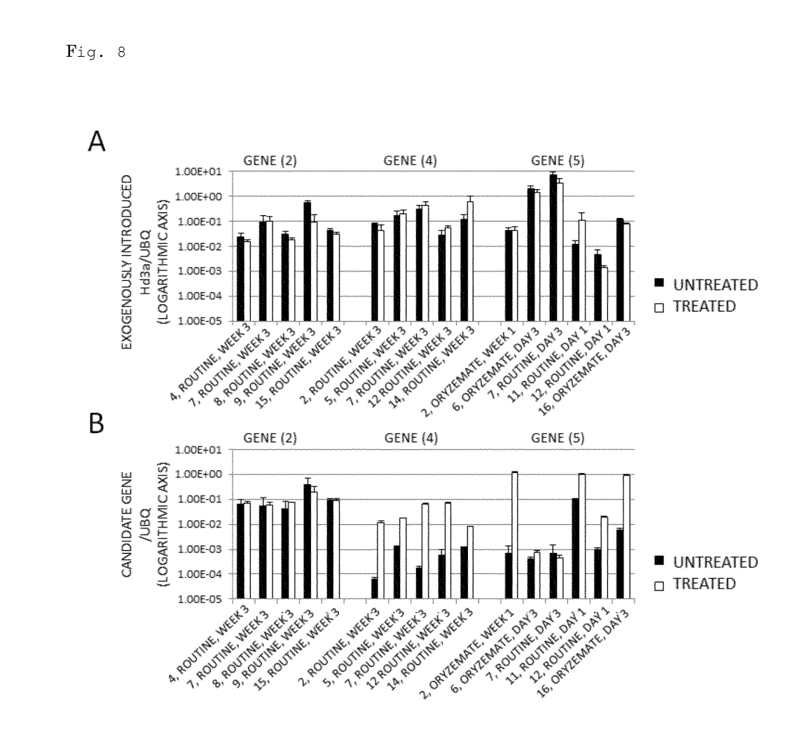

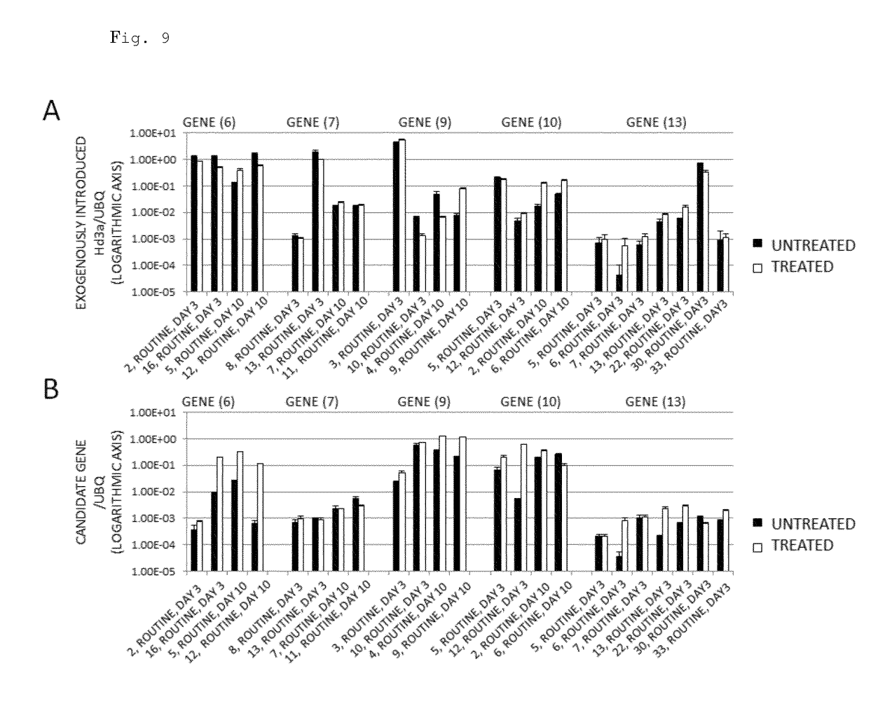

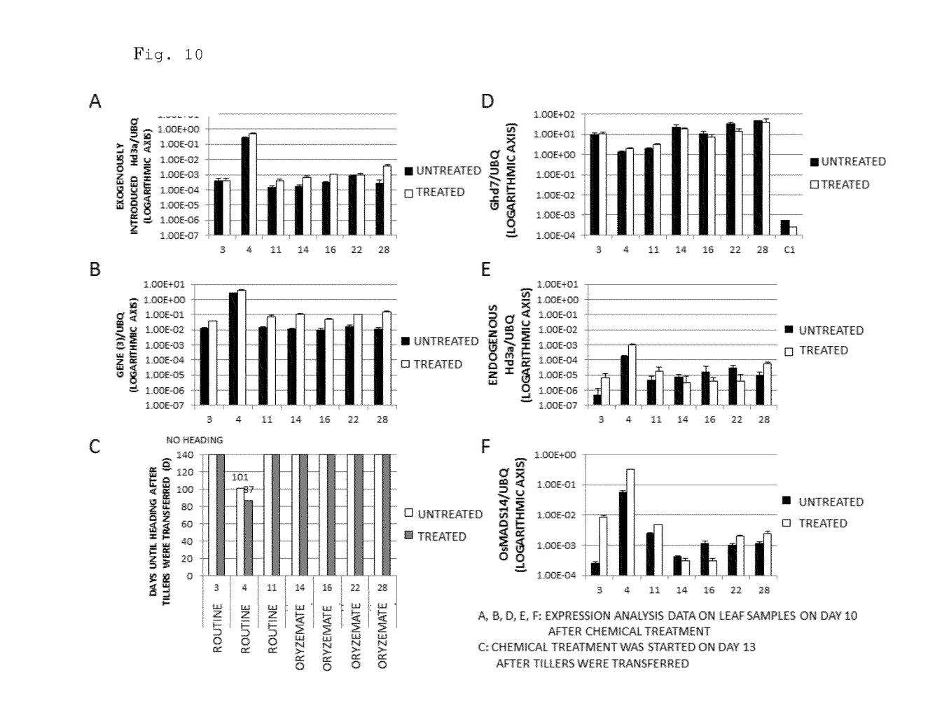

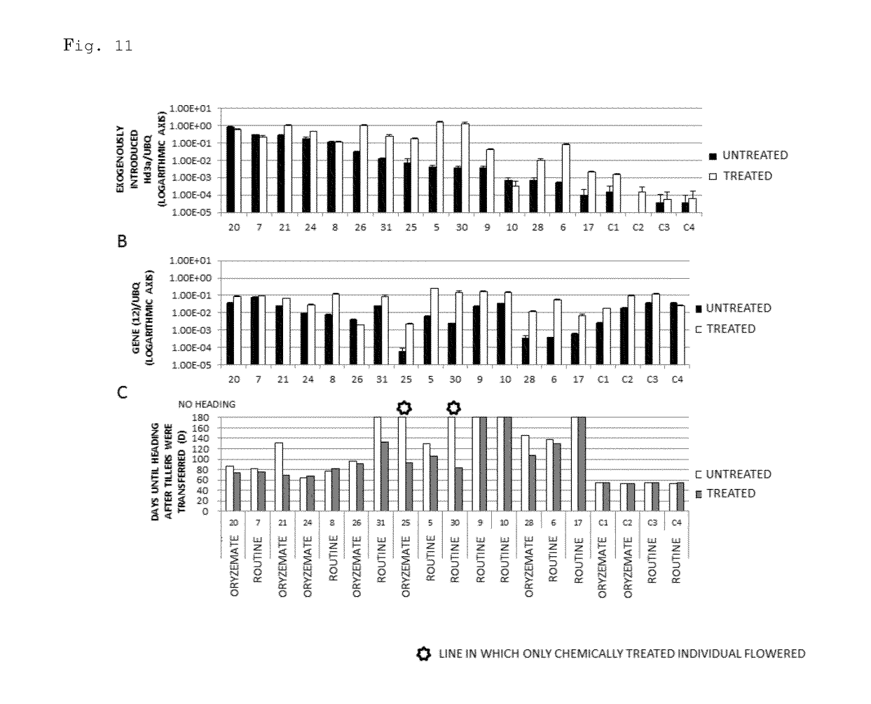

The transgenic lines obtained by using the variety of gene promoters were examined for the endogenous expressions of the genes corresponding to the gene promoters used. As a result, significant increases at the expression level of, particularly, the genes (3), (4), (5), (6), and (12) were observed in the individuals in the treated plot in comparison with the individuals in the untreated plot. Lines exhibiting 10-fold or more of such increases were also confirmed (FIGS. 8, 9, 10B, 11B). Moreover, examined were the expressions of Hd3a (exogenously introduced Hd3a) introduced in such a manner that Hd3a was ligated to the corresponding gene promoters. The result confirmed that there was a difference in the presence or absence of florigen expression inductions by the chemical treatment among lines of the transformants obtained by using the promoters of the genes (3) and (12), and that some lines were similar to each other in the presence or absence of induced expressions of the candidate genes themselves by the chemicals utilizing the promoters (FIGS. 10AB, 11AB). Particularly, in the case of using the promoter of the gene (3), the difference in the inductions was only an order of magnitude among the lines confirmed to be induced. Meanwhile, several lines (T0 individuals #5, 6, and 30 in FIG. 11A) of the transformants obtained by using the promoter of the gene (12) exhibited 100-fold or more of increases at the expression level when exogenously introduced Hd3a was induced.

The difference in the basic expression level observed in the lines of all the produced transformants when exogenously introduced Hd3a was not induced (the expression level of exogenously introduced Hd3a as seen in the untreated individuals) was presumably due to the positional effect because the introduced genes were randomly inserted into the chromosomes in the gene introduction by the Agrobacterium method. Additionally, the difference was presumably influenced by the difference in the number of copies of the introduced gene inserted in the chromosomes, too.

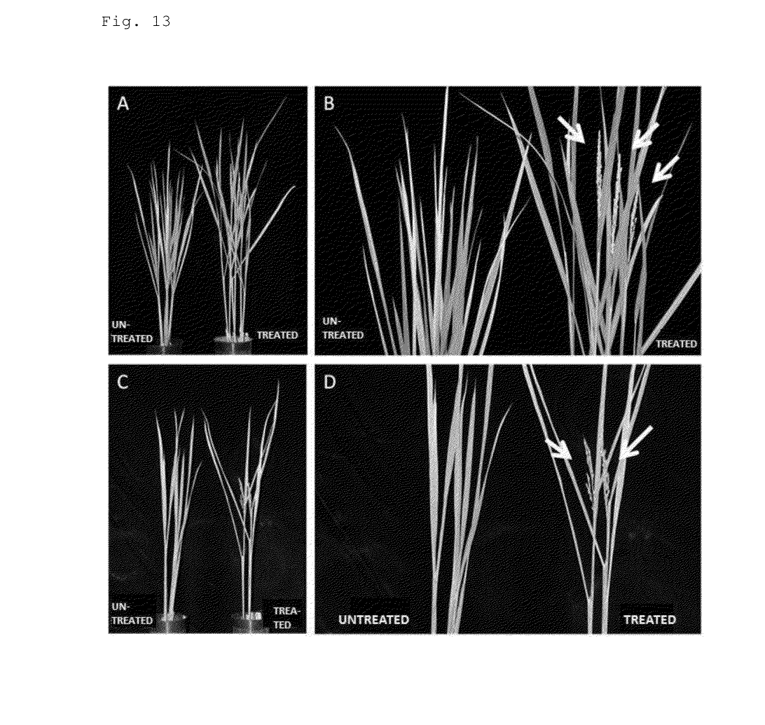

Next, the flowering (heading) status was examined. As a result, most of the lines produced this time as the transformants obtained by using the gene (3) promoter did not flower regardless of the treatment/untreatment with the chemicals. In one line, the heading was observed 14 days earlier in a chemically treated individual (FIG. 10C); nevertheless, the flowering was observed also in the untreated individual. On the other hand, regarding the transformants obtained by using the gene (12) promoter, it was observed that treated individuals flowered earlier than untreated individuals in many lines. It was observed that some lines exhibited the difference by one month or more, and also that only treated individuals flowered in several lines (four or more lines including #25 and #30 in FIG. 11C) (FIG. 13).

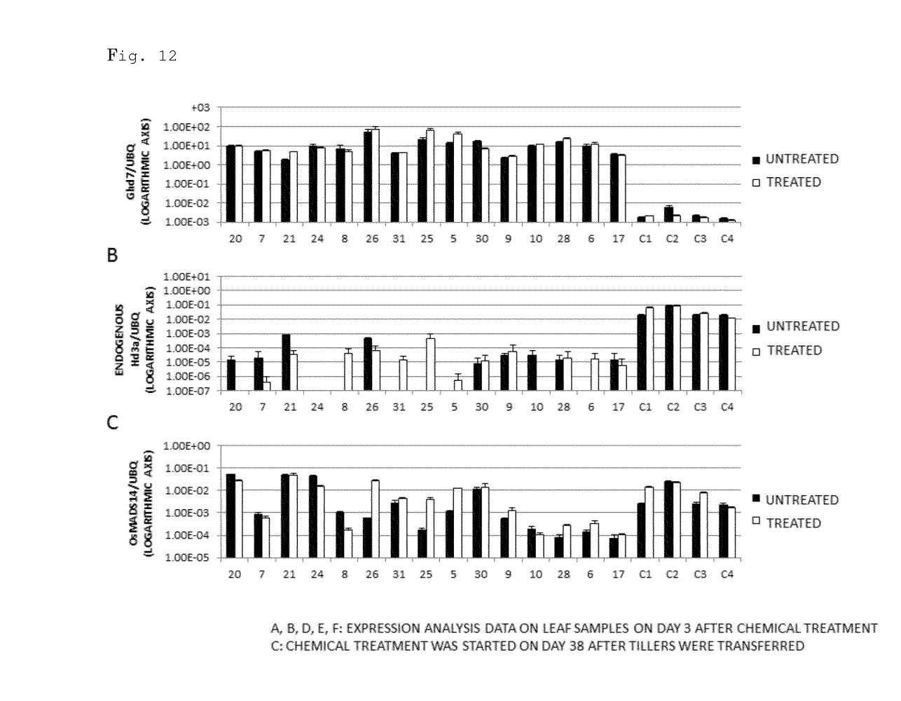

Meanwhile, in the present transformants, Ghd7 was co-introduced together with the corn-derived ubiquitin promoter which was ligated thereto and capable of constitutive expression at a high level. As a result of the expression analysis on the transformants obtained by using the promoters of the genes (3) and (12), the Ghd7 expression was exhibited at a high level as intended, in accordance with which it was also confirmed that the expression of endogenous Hd3a was suppressed to a low level (FIGS. 10DE, 12AB). Further, when examined by the genetic analysis, the expression of OsMADS14 believed to function downstream of Hd3a/RFT1 basically corresponded to the expression variation of exogenously introduced Hd3a (FIGS. 10F, 12C). Although some lines were observed to exhibit variations not completely consistent to such a behavior, this was presumably due to the feedback control of the transcription of OsMADS14, and the like, which were not been completely understood at present. OsMADS14 and OsMADS15, which are homologous genes of AP1/FUL involved in flower bud differentiation, are activated by Hd3a/RFT1 in a shoot apical meristem, and function downstream thereof; meanwhile, there is also a report that the transcription is activated upstream of Hd3a/RFT1 in leaves. It is also considered that OsMADS14 and OsMADS15 activate the transcription of Hd3a/RFT1, and vice versa (Komiya et al., Development. 2008; 135, 767-774, Kobayashi et al., Plant Cell. 2012; 24 (5): 1848-59). From the foregoing, the transcription control of OsMADS14 has not been fully understood yet. Meanwhile, there is also a report that Hd3a/RFT1 ortholog FT of Arabidopsis thaliana has a negative feedback control to suppress its own transcription (Liu et al., PLoS Biol. 2012; 10 (4): e1001313). Accordingly, there may be a possibility that the expression inconsistency of some lines observed in this Example was due to a negative feedback control by OsMADS14 itself at a transcription level.

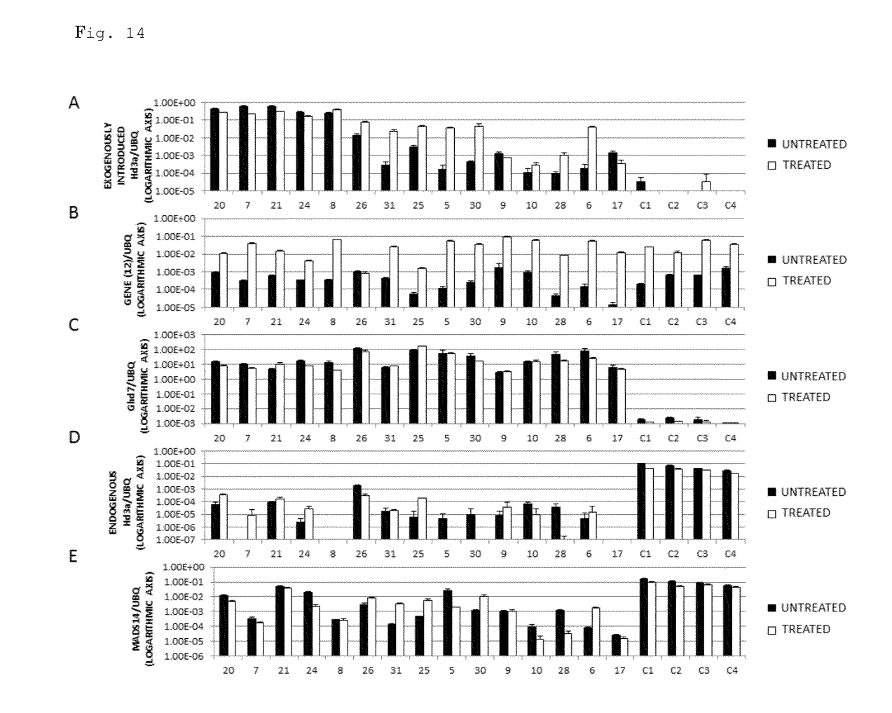

Additionally, an expression analysis was conducted again on the transformants obtained by using the gene (12) promoter, in many lines from which the flowering inductions were observed (the analysis used leaf samples thereof on Week 2 after the chemical treatment). As a result, reproducible expression patterns were confirmed in the lines (FIG. 14).

In this Example, it was possible to produce lines exhibiting 100-fold or more of increases at the expression level of exogenously introduced Hd3a when the expression was induced in comparison with when not induced, and it was also possible to produce transgenic lines whose flower bud differentiation was controllable by a chemical such that the transgenic lines were not merely observed to be flowering inducible, but also actually flowered earlier by one month or more when the plants were planted in the treated plot, and did not flower when untreated.

### Example 5

### Morphological Examination of Head

A morphological examination was conducted on the head of the transgenic line obtained by using the promoter of the gene (12) described in Example 4.

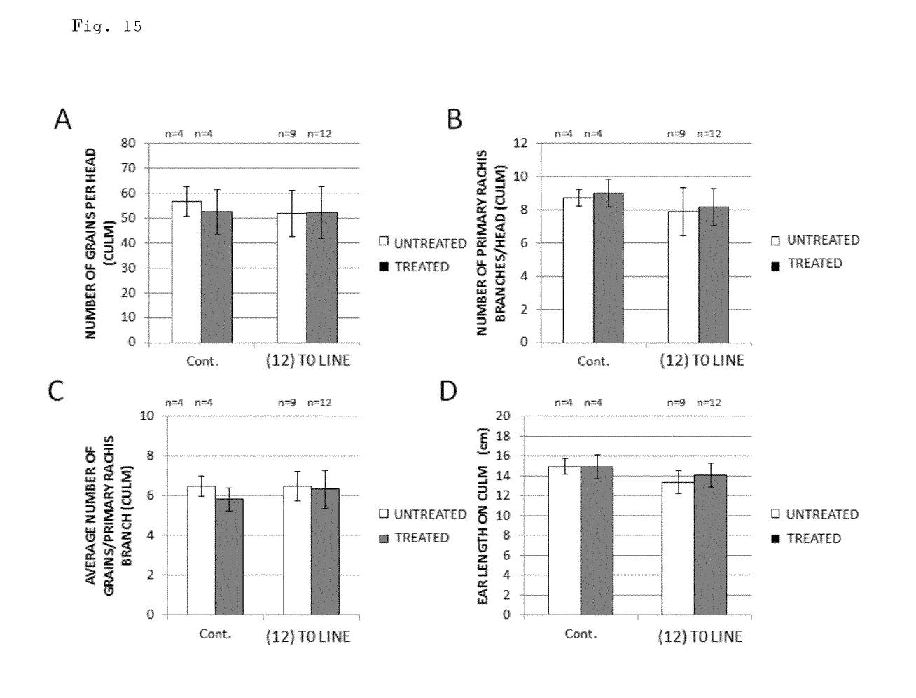

FIG. 15 shows the result of measuring the number of grains per head, the number of primary rachis branches, an average number of grains per primary rachis branch (average number of grains/primary rachis branch), and ear length of each head on a culm of the transformants obtained by using the gene (12) promoter ((12) T0 line). In comparison with a control line (Cont.: a line not having both of the Ghd7 and Hd3a genes introduced therein), no clear difference was observed in any of the number of grains per head, the number of primary rachis branches, the average number of grains/primary rachis branch, and the ear length of the transformants obtained by using the gene (12) promoter. No clear morphological abnormality was observed in the head (FIG. 15).

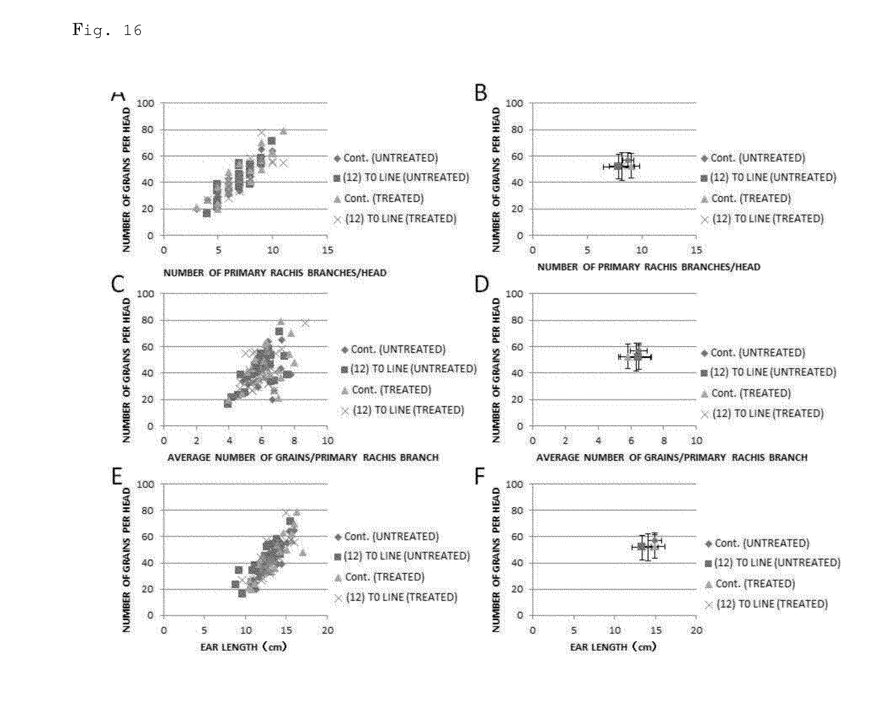

Moreover, all the matured ears of the individuals after the heading were collected (collected on Day 40 after the heading of the untreated individuals of the #28 line of Example 4 in FIG. 11C), and the same examination was conducted. As a result, no clear morphological abnormality was observed in the head (FIG. 16).

### Example 6

### Flowering Induction Test on Progenies

Progenies (T1 generation) of the transformants obtained by using the promoter of the gene (12) were subjected to a flowering induction test by a plant activator treatment.

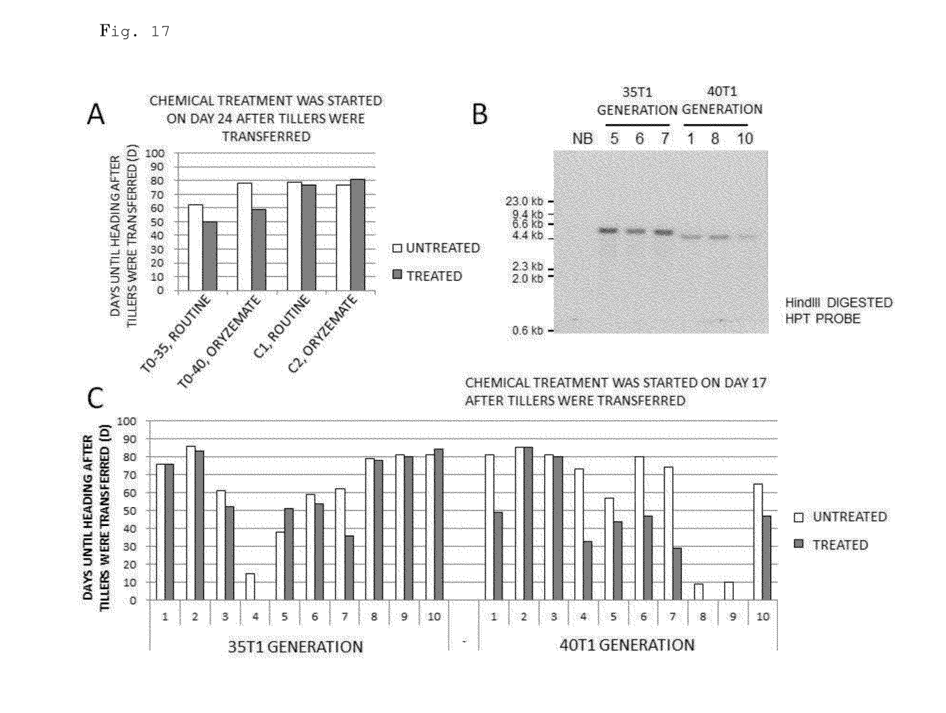

T1 segregation generations of T0-35 and T0-40 (FIG. 17A) in the transformation generation (T0 generation) from which the flowering inductions by the plant activator had been observed were seeded. Tillers of the plants on Day 40 after the seeding were divided for the treatment/untreatment with the chemicals and then transferred. On Day 17 after transferred and grown, individuals on a treated plot were subjected to a spray treatment with Routine 1 kg granule (Bayer CropScience AG) (0.5 g/individual, treated again in the same amount 5 days later) (FIG. 17C). A T1 individual #4 in the T0-35 line and T1 individuals #8 and #9 in the T0-40 line were not divided because flag leaf/internode elongation was observed on Day 40 after the seeding. The plants were grown in a growth chamber (long-day conditions: 14.5 hours of the light period: 9.5 hours of the dark period, temperature setting: 28° C. during the light period: 25° C. during the dark period, illumination: a metal-halide lamp of 500 μE).

When the tillers were divided, the genomic PCR analysis was conducted to confirm the introduced gene. The introduced gene was not detected in five individuals (T1-1, T1-2, T1-8, T1-9, T1-10) in the T1 generation of the T0-35 line and two individuals (T1-2, T1-3) in the T1 generation of the T0-40 line, and the segregation was observed in the T1 population of the two lines. Moreover, three individuals from each of the T1 generations of the T0-35 line and the T0-40 line were subjected to a genomic Southern blotting analysis. As a result, although the sizes were different between the individuals derived from the T0-35 line and the individuals derived from the T0-40 line, single bands were respectively detected from all the T1 individuals, revealing that each of the parental lines had a single copy of the introduced gene (FIG. 17B). Thus, since the introduced gene is to be inherited to the next generation at a ratio of 1 (homo):2 (hetero):1 (no introduced gene) according to Mendel's laws, T1-4 of the T0-35 line and T1-8 and T1-9 of the T0-40 line observed to have flag leaves when the tillers were divided were expected to be lines each having the introduced gene in a homozygous state.

The genomic DNA used in the genomic PCR analysis was extracted according to the simple method described in Example 2. The introduced gene was confirmed by amplifying the hygromycin resistance gene (Hpt) by PCR using a primer P35S1 (5′-TCCACTGACGTAAGGGATGA-3′/SEQ ID NO: 84) and a primer Nos3 (5′-ATCAGCTCATCGAGAGCCT-3′/SEQ ID NO: 85).

The total genomic DNA used in the genomic Southern blotting analysis was extracted from leaves of the plant by the CTAB method. While being frozen with liquid nitrogen, the leaves were ground into a powder form using a pestle and a mortar. The resultant was then incubated (55° C., 60 minutes) in 5 ml of preheated 2×CTAB buffer (2% CTAB, 1.4 M NaCl, 100 mM Tris-HCl (pH 8.0), 20 mM EDTA; 75 l of 2-mercaptoethanol was added when used). Subsequently, an equal amount of a CIA solution (chloroform:isoamyl alcohol=24:1) was added thereto, stirred with a rotator for 30 minutes, and centrifuged (8000 rpm, 20 minutes, room temperature). The supernatant was subjected to isopropanol precipitation. After washing with 70% ethanol, the resultant was dissolved in 400 μl of a TE buffer (10 mM Tris-HCl, 1 mM EDTA (pH 8.0)), and subjected to an RNase treatment (1 μl of RNase G.S (10 mg/ml RNase, Wako) was added and incubated at 37° C. for 60 minutes). A phenol-chloroform treatment was performed, and the supernatant was subjected to ethanol precipitation. Thereafter, the resultant was dissolved in 50 μl of a TE buffer. Thus, the total genomic DNA was extracted.

The DNA was blotted according to the conventional method described in Molecular cloning: A Laboratory Manual 3rd Edition (ed. Sambrook J. and Russell D. W., Cold Spring Harbor Laboratory Press, Cold Spring Harbor, N. Y., 2001). First, the total genomic DNAs (3 μg) of Nipponbare and the transformant to be analyzed were each treated with HindIII, and separated using 0.8% agarose gel by electrophoresis (voltage of 50 V, 120 minutes). After the electrophoresis, the gel was subjected to treatments of depurination (shaken in a 0.25 M solution of hydrochloric acid for 15 minutes), alkali denaturation (shaken in an alkali denaturation solution (1.5 M NaCl, 0.5 M NaOH) for 45 minutes), and neutralization (shaken in a neutralization solution (1.0 MTris-HCl (pH 7.4), 1.5 MNaCl) for 45 minutes). Thereafter, the resultant was blotted (for 16 hours or more) on a nylon membrane (Nylon Membranes positively charged, Roche) using a 20×SSC solution (3 M NaCl, 300 mM sodium citrate), after a blotting stage was set according to the conventional method. To cross link and fix the DAN to the nylon membrane after the blotting, a UV crosslinker was used. The DNA probe preparation, DNA hybridization, and signal detection were carried out based on DIG System (Roche) according to the manual. The detected signals were exposed to an X-ray film (GE Healthcare), and the image was inputted into a PC with a scanner for the analysis.

As a result of the flowering examination on the T1 generations, it was observed that three individuals (T1-3, T1-6, T1-7) in the T0-35 line and five individuals (T1-1, T1-4, T1-5, T1-6, T1-7, T1-10) in the T0-40 line flowered significantly earlier when treated than when untreated. This revealed that the flowering was induced by a chemical in the progenies (FIG. 17C).

This Example demonstrated that the introduced trait was stably inherited to the progeny.

### Example 7

### Flowering Induction Test in Field

The transformants obtained by using the gene (12) promoter from which the flowering induction by the chemical treatment in the experimental environment had been confirmed were subjected to the flowering induction test in the open air (cultivated in a field in South Korea).

To an untreated plot and a treated plot where a chemical was sprayed which were provided in such a way that one plot was not contaminated with the plant activator agent in the other plot, ten individuals of each of a control line (Nipponbare) and the T1 segregation generation of the line (the T0-40 line described in Example 5) having one copy of the introduced gene in a hemizygous state were transferred (seeded on 2012 May 5, the seedlings were transplanted on 2012 Jun. 5). On Week 3 (2012 Jul. 26) after the transferring, the plants in the treated plot were subjected to the spray treatment with probenazole 6% granule (Bayer CropScience AG) (45 g/m2 at one time, treated every 5 days, three times including the initial time).

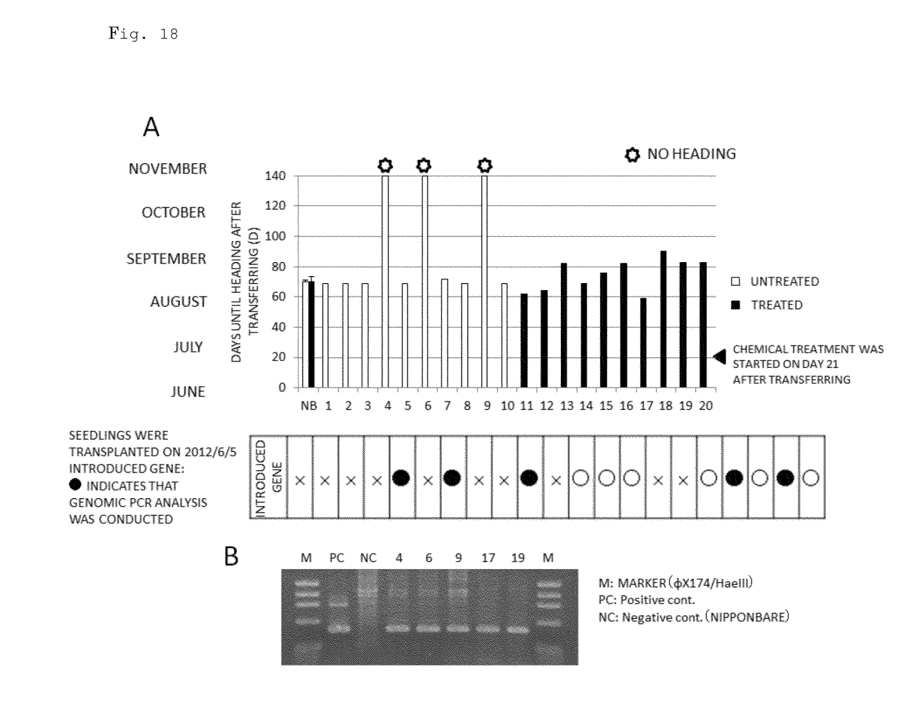

As a result of examining the flowering status after the chemical spray treatment in the field, all the individuals in the control line Nipponbare flowered at the same timing (from the middle of August to the last half of the month) regardless of the treatment/untreatment with the chemical. On the other hand, all the transformant individuals in the treated plot flowered, but three individuals in the untreated plot did not flower even in November (on 2012 Nov. 1) (FIG. 18A).

The genomic DNA was extracted from leaves of each of the three individuals (#4, #6, #9) which did not flower in the untreated plot and two individual (#17, #19) which flowered in the treated plot. The result of the PCR analysis confirmed that all of these individuals had the introduced gene (FIG. 18B). These results showed that the flowering of the transgenic line obtained by using the gene (12) promoter was inducible by the plant activator treatment in the open air, too.

The genomic DNA was extracted from the leaves using FTA Plant Kit (Whatman) according to the manual. In the PCR analysis, HPT_probe_F (5′-GTCCGTCAGGACATTGTTGGAGCCGAAA-3′/SEQ ID NO: 86) and HPT_probe_R (5′-GCGTGGATATGTCCTGCGGGTAAATAGCTG-3′/SEQ ID NO: 87) were used as the primers.

This Example demonstrated that the present invention was actually utilizable not only in an experimental environment but also in an open air environment.

### Example 8

### Example of Using Flowering-Time Control DNA Cassette in Which Translational Enhancer was Introduced

Transformants (Nipponbare background) were produced using the flowering-time control plasmid pRiceFOX/Ubi:Ghd7/Gate:Adh5′UTR:Hd3a with the translational enhancer (FIG. 2B) for the above-described promoter of the gene (12) suitable for the flowering induction, and subjected to the flowering induction test.

Replicate individuals for the treatment and the untreatment with the plant activator were prepared by dividing tillers of each line of the transformants, and transferred to and grown in a growth chamber (long-day conditions: 14.5 hours of the light period: 9.5 hours of the dark period, temperature setting: 28° C. during the light period: 25° C. during the dark period, illumination: a metal-halide lamp of 500 ρE). The chemical spray treatment was started on the plants for the treatment on Day 25 after the plants were transferred and grown. As the chemical, Routine 1 kg granule (Bayer CropScience AG) was used, and the plants were treated every 5 days with the chemical in an amount of 0.5 g/individual at one time, hence treated three times including the initial time.

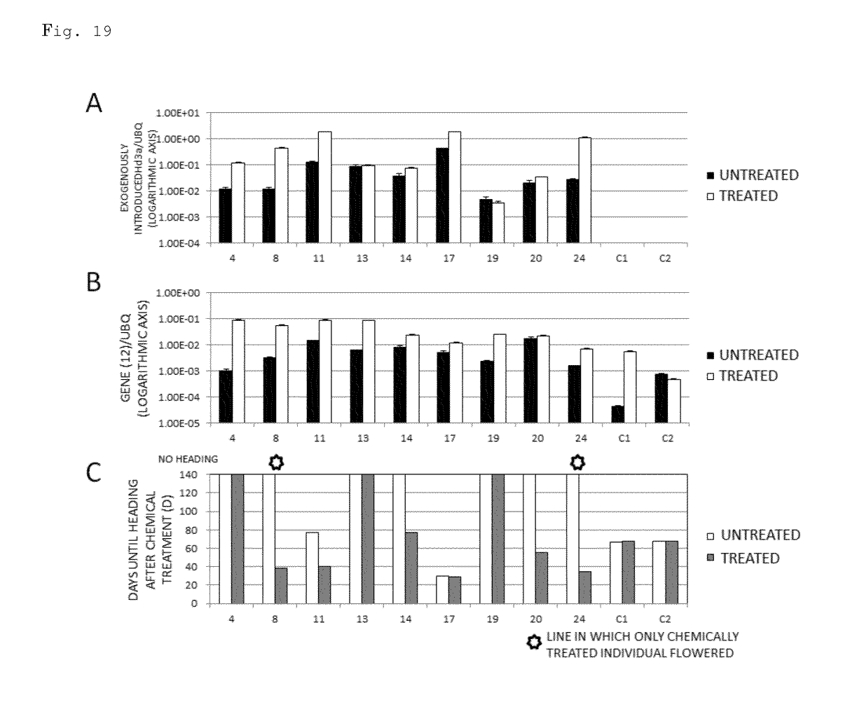

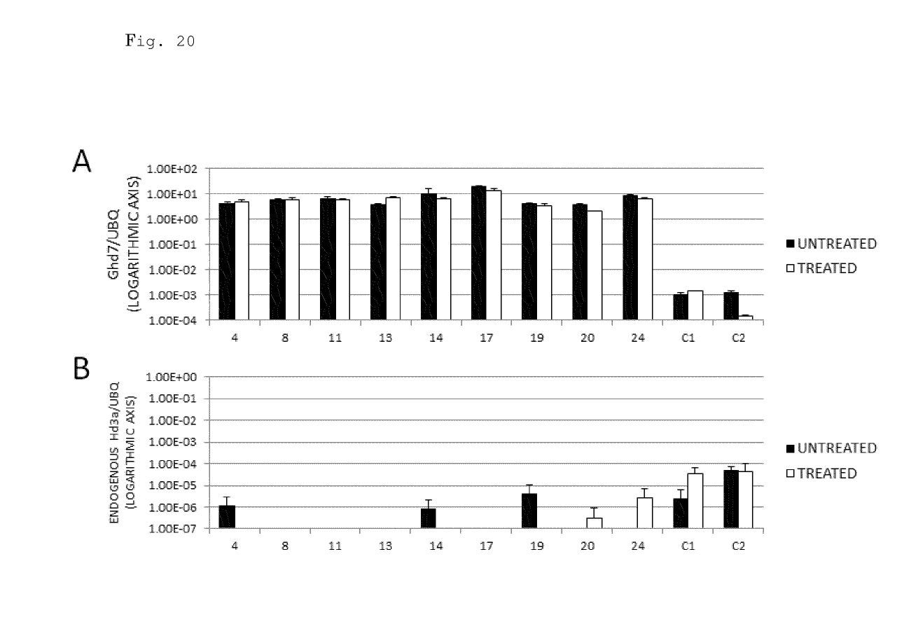

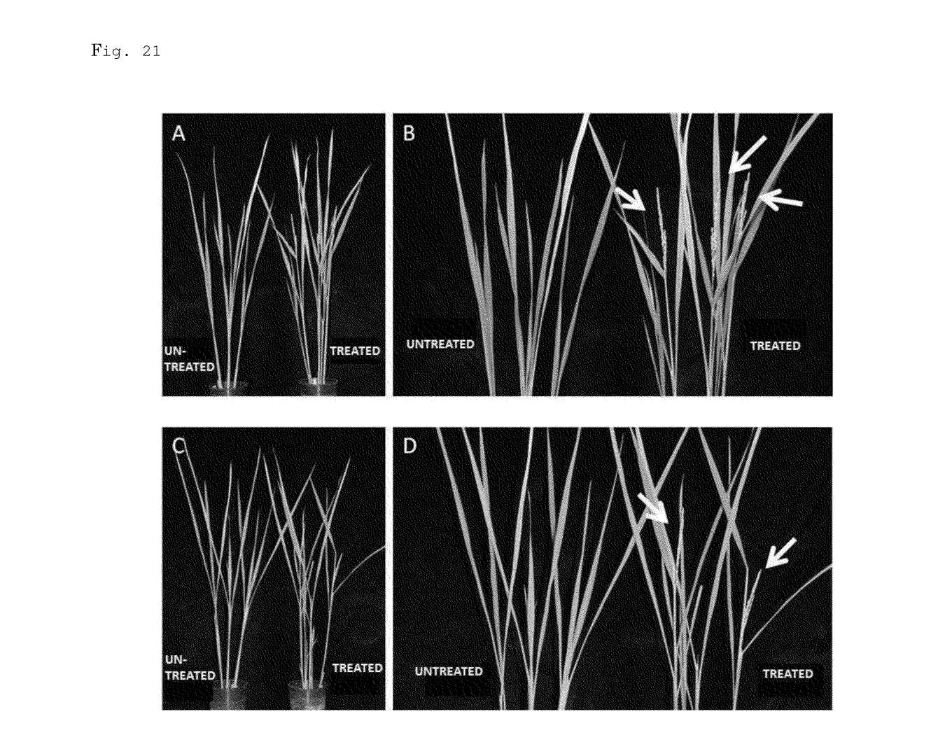

As a result of the flowering induction test, it was observed that the chemically treated individuals flowered earlier than the untreated individuals in multiple lines (FIG. 19C). Particularly, the chemically treated individuals in the #8 and #24 lines produced the ears on Day 38 and Day 35, respectively, but no heading was observed from the untreated individuals in both of the lines (FIGS. 19C, 21). Moreover, on Day 3 after the chemical treatment, leaves were collected to examine the expression by the quantitative RT-PCR analysis. As a result, the expression induction of the gene (12) and also the expression induction of exogenously introduced Hd3a were confirmed in the lines from which the significant flowering induction had been observed (FIG. 19AB). In addition, it was also confirmed that the Ghd7 gene was constitutively expressed at a high level and that the expression of the endogenous Hd3a gene was suppressed at the same time (FIG. 20AB).

In this test also, multiple lines were obtained which flowered only by a chemical treatment.

The RNA extraction, the cDNA synthesis, and the real-time PCR accompanying the quantitative RT-PCR analysis were carried out according to the methods described in Example 4. Additionally, the quantitative RT-PCR analysis on each gene was conducted using primers and probes shown in Table 4.

TABLE 4
Primer sequences and Taq man probe sequences used in the quantitative RT-PCR

| Gene | Primer/probe name | Sequence (5′-3′) |
|---|---|---|
| gene (1) | Os01g0567200_real_fw | TGCCACCATTCGAGTTCTTCA/SEQ ID NO: 88 |
| | Os01g0567200_real_rv | CCGAACAACAAACCTTGCATG/SEQ ID NO: 89 |
| gene (2) | Os03g0629800_real_fw | CAGACCTCCGTTTTTGTGCAG/SEQ ID NO: 90 |
| | Os03g0629800_real_rv | GCGATAATGCCGTGACGAAT/SEQ ID NO: 91 |
| gene (3) | Os04g0556400_real_fw | GGCGGATAATCCGAATTTCAC/SEQ ID NO: 92 |
| | Os04g0556400_real_rv | TGGATAAGGTGGACGTGGATG/SEQ ID NO: 93 |
| gene (4) | Os07g0687400_real_fw2 | GGTAGACAACACTATTACTCC/SEQ ID NO: 94 |
| | Os07g0687400_real_rv2 | CCTGCAGTTTCAACTAGAC/SEQ ID NO: 95 |
| gene (5) | Os12g0458100_real_fw2 | CACAATTGAACTCGTCCGGA/SEQ ID NO: 96 |
| | Os12g0458100_real_rv2 | TGTACGGTTTTTCACGCCAC/SEQ ID NO: 97 |
| gene (6) | Os04g0339000_real_fv | AAGCTGCCCAATGGAATGTTG/SEQ ID NO: 98 |
| | Os04g0339000_real_rv | TGGAAGCAATGTGAGTGACCG/SEQ ID NO: 99 |
| gene (7) | Os04g0371600_real_fw3 | G2ACTTTTTTCCCAATTCCCC/SEQ ID NO: 100 |
| | Os04g0371600_real_rv3 | TGAAAGCACACGGAGACCTTG/SEQ ID NO: 101 |
| gene (8) | Os06g0671300_real_fw | TTGCACATGCCACACTCGA/SEQ ID NO: 102 |
| | Os06g0671300_real_rv | CCCGAATTCCTCTTCCATGTC/SEQ ID NO: 103 |
| gene (9) | Os07g0489000_real_fw | TCCAGCCCCGATCACAATAGT/SEQ ID NO: 104 |
| | Os07g0489000_real_rv | CGGTACGTAATTTGGCATCGC/SEQ ID NO: 105 |
| gene (10) | Os11g0514400_real_fv3 | TCACTGCCCTTCTTGCTTTTG/SEQ ID NO: 106 |
| | Os11g0514400_real_rv3 | CGCCAGCACATTGTTGATGT/SEQ ID NO: 107 |
| gene (11) | Os01g0108500_real_fw | TTTCCCACCAGCTCATTCCA/SEQ ID NO: 108 |
| | Os01g0108500_real_rv | TCACCGGACTCAGCAAGAGAA/SEQ ID NO: 109 |
| gene (12) | Os01g0108400_real_fw2 | TGCTCCATGTCCAAGATGCA/SEQ ID NO: 110 |
| | Os01g0108400_real_rv2 | GCAGCGCGATGATGTGATACT/SEQ ID NO: 111 |
| gene (13) | Os08g0428200_real_fw2 | AGATTGCGTCTCATTTGCCTG/SEQ ID NO: 112 |
| | Os08g0428200_real_rv2 | CCGTGTTCTTTCTCTCTGCGT/SEQ ID NO: 113 |
| exogenously | OKD_U | gcaagaggtgatgtgctacg/SEQ ID NO: 114 |
| introduced | | |
| Hd3a (Kasalath | | |
| cultivar Hd3a) | | |
| | OKD_KAS_L2 | TCGAGCTCGGTACCCTCGTT/SEQ ID NO: 115 |
| exogenously | OKD_U | gcaagaggtgatgtgctacg/SEQ ID NO: 116 |
| introduced | | |
| Hd3a into the | | |
| line obtained | | |
| by using the | | |
| gene (3) promoter | | |
| | (3) OKD_KAS_L2 | AGAATCAGGGTACCCTCGTT/SEQ ID NO: 117 |
| endogenous Hd3a | OKD_U | gcaagaggtgatgtgctacg/SEQ ID NO: 118 |
| | OKD_NIP_L | gggatcatcgttagctcggg/SEQ ID NO: 119 |
| Ghd7 | forward primer | GTACGCGTCCAGAAAAGCT/SEQ ID NO: 120 |
| | reverse primer | TTGGCGAAGCGACCTCTC/SEQ ID NO: 121 |
| | Ghd7 Taq man probe | TGCCGAGATGAGGCCCCGA/SEQ ID NO: 122 |
| OsMADS14 | forward primer | CCACCAAGGGCAAGCTCTAC/SEQ ID NO: 123 |
| | reverse primer | AGCGCTCATAACGTTCAAGGA/SEQ ID NO: 124 |
| | OsMADS14 Tag man | AGTACGCCACCGACTCATGTATGGACAAA/ |
| | probe | SEQ ID NO: 125 |
| UBQ | forward primer | GAGCCTCTGTTCGTCAAGTA/SEQ ID NO: 126 |
| | reverse primer | ACTCGATGGTCCATTAAACC/SEQ ID NO: 127 |
| | UBQ Taq man probe | TTGTGGTGCTGATGTCTACTTGTGTC/ |
| | | SEQ ID NO: 128 |
| corn gene (12) | GRMZM2G169947_real_F | CCCTCATCCCGAAAGAACACTA/SEQ ID NO: 129 |
| ortholog | | |
| (GRMZM2G169947) | | |
| | GRMZM2G169947_real_R | TCCACCCTCTCCTTGAGTCTCT/SEQ ID NO: 130 |
| corn UBQ (Planta. | ZmUbi1_F | gtttaagctgccgatgtgcctg/SEQ ID NO: 131 |
| 2008 May; 227 | | |
| (6): 1377-88) | | |
| | ZmUbi1_R | gacacgactcatgacacgaacagc/SEQ ID NO: 132 |

### Example 9

### Example of Applying Flowering-Time Control DNA Cassette to Feed Rice Cultivars

Next, the application of the present invention to rice cultivars other than Nipponbare was tested. The flowering-time control plasmid (pRiceFOX/Ubi:Ghd7/Gate:Hd3a, FIG. 2a) in which the promoter of the gene (12) was introduced was used to transform feed rice cultivars Tachisugata and Kitaaoba, and the transformants thus produced were subjected to the flowering induction test.

Replicate individuals for the treatment and the untreatment with the plant activator were prepared by dividing tillers of each line of the transformants, and grown in a growth chamber (long-day conditions: 14.5 hours of the light period: 9.5 hours of the dark period, temperature setting: 28° C. during the light period: 25° C. during the dark period, illumination: a metal-halide lamp of 500 μE). The chemical treatment was performed on individuals for the treatment on Day 66. As the chemical, Routine 1 kg granule (Bayer CropScience AG) was used, and the plants were treated every 5 days with the chemical in an amount of 0.5 g/individual at one time, hence treated three times in total.

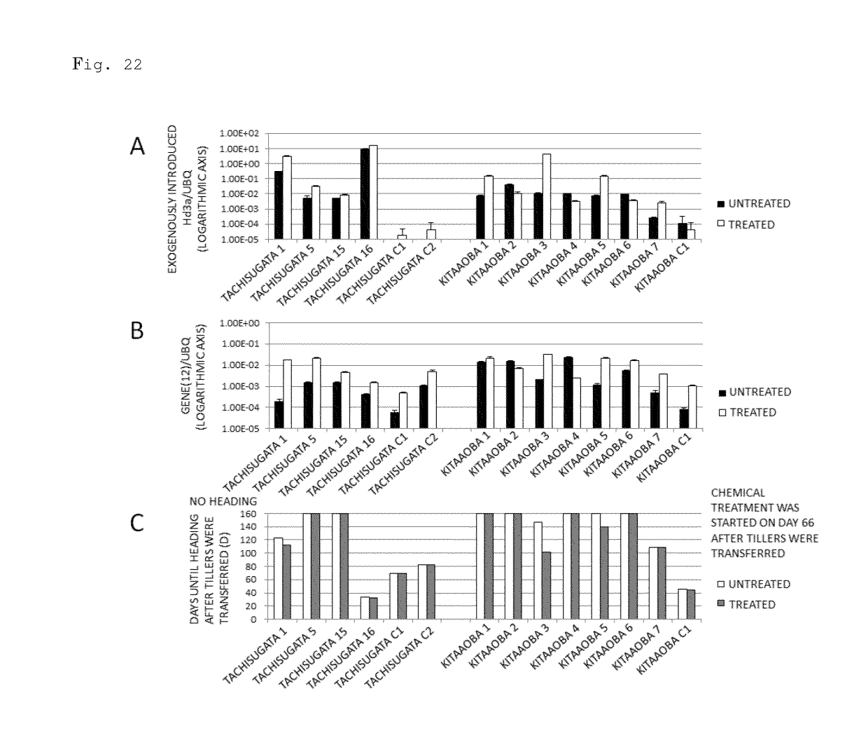

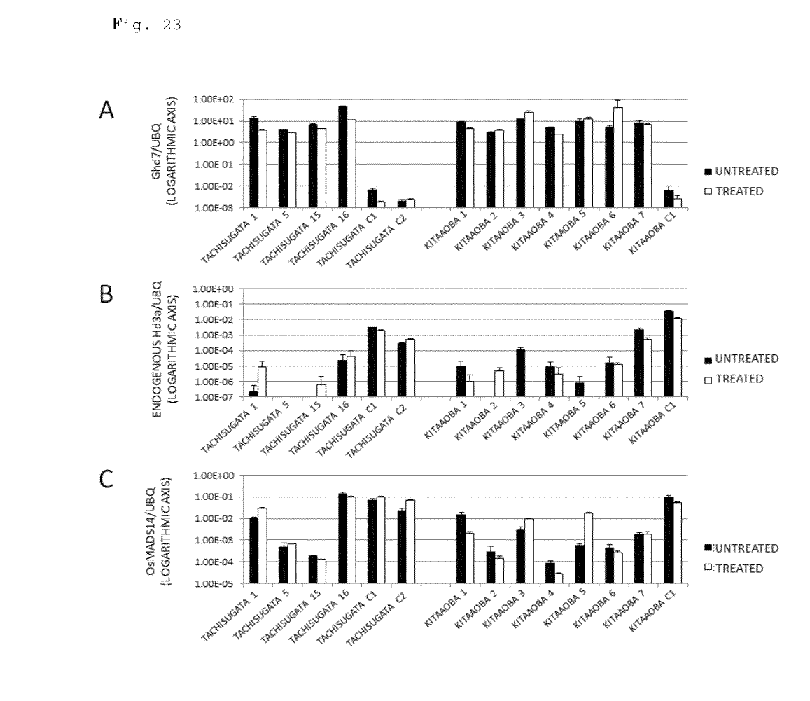

The expression in leaves (leaf blades on Day 3 after the chemical treatment) was analyzed by the quantitative RT-PCR analysis. As a result, the expression induction of the gene (12) and also the expression induction of exogenously introduced Hd3a were confirmed in multiple lines of the transformants of each of the Tachisugata- and Kitaaoba-background cultivars (FIG. 22AB). Depending on the lines, ten-fold to several hundred-fold or more inductions of exogenously introduced Hd3a were observed. Moreover, the comparison between control lines not having both of the introduced genes Ghd7 and Hd3a (Tachisugata C1, Tachisugata C2, Kitaaoba C1) and the transformant lines of the corresponding background cultivars confirmed that the Ghd7 gene was constitutively expressed at a high level while the expression of endogenous Hd3a was suppressed in the transformants of both of the Tachisugata- and Kitaaoba-background cultivars (FIG. 23ABC). Next, the flowering status was examined. Asa result, it was observed that the treated individuals flowered earlier than the untreated individuals in some lines; Tachisugata 1 flowered earlier by 10 days or more, and Kitaaoba 3 flowered earlier by one month or more. The flowering induction was confirmed in the transformants of both of the background cultivars, too (FIG. 22C). In both of the two lines (Tachisugata 1 and Kitaaoba 3), the expression induction of OsMADS14 believed to genetically function downstream of Hd3a was also confirmed (FIG. 23C).

This Example demonstrated that the present invention was applicable regardless of the cultivar in the case of rice.

### Example 10

### Re-Flowering Induction Test on Flowering-Induced Lines

Examples so far have stated that multiple lines were produced whose flowering is inducible by a chemical treatment, but the plants would not flower unless treated. In this Example, tillers of the untreated individuals of the T0-30 line described in Example 4 (FIG. 11C) as well as the T0-8 line and the T0-24 line described in Example 8 (FIG. 19C) in a non-flowering state were divided again for the treatment/untreatment with the chemical, and subjected to the flowering induction test again.

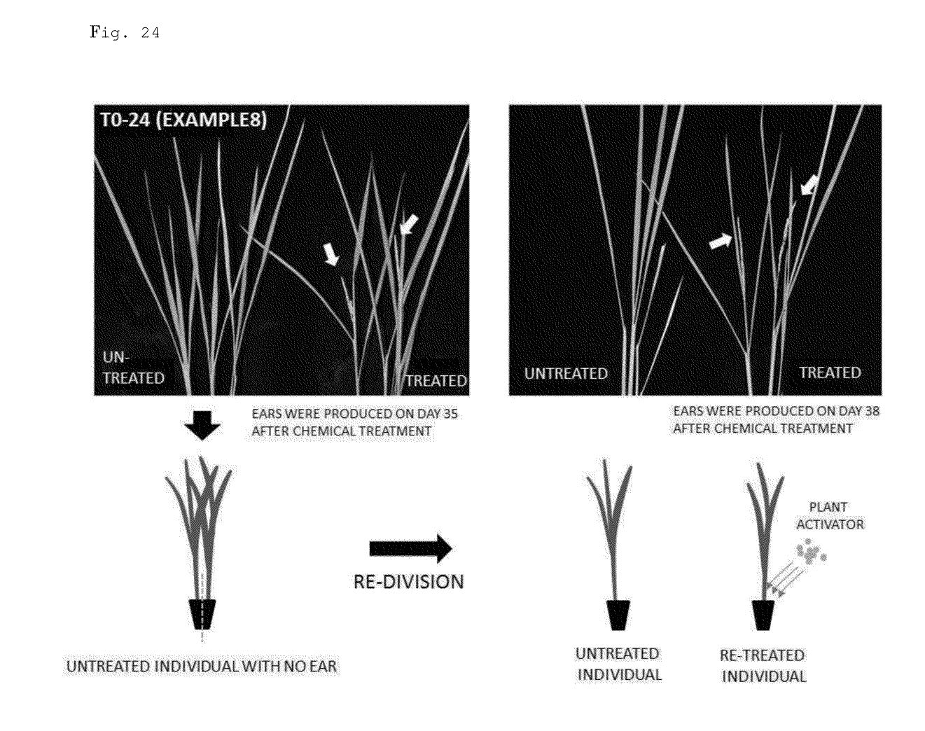

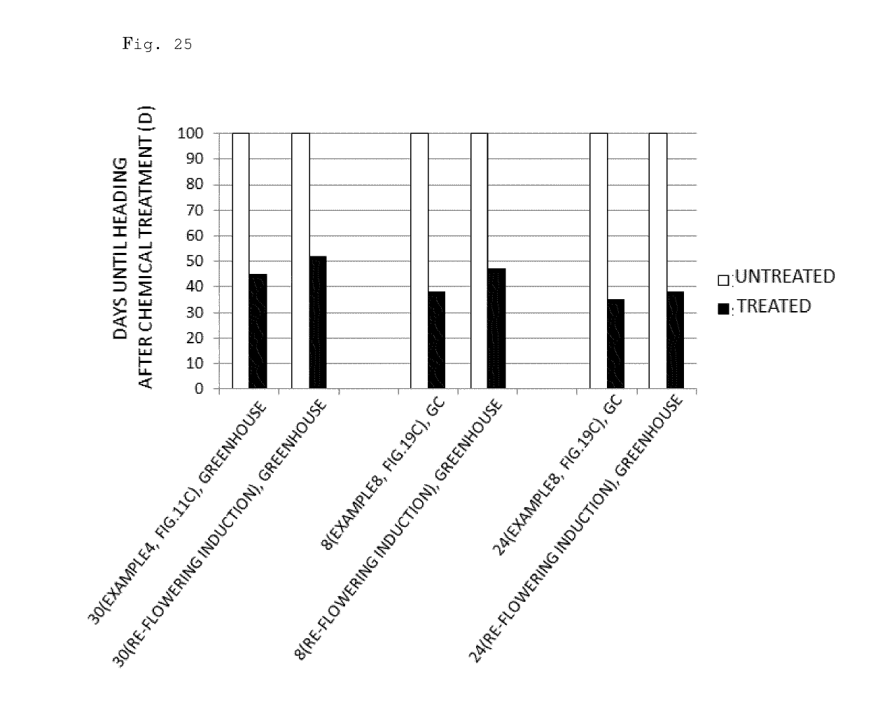

The tillers of each line of the untreated individuals after the first flowering induction test were divided and grown in a glass greenhouse (greenhouse) or a growth chamber (GC) (long-day conditions: 14.5 hours of the light period: 9.5 hours of the dark period, temperature setting: 28° C. during the light period: 25° C. during the dark period, illumination: a metal-halide lamp of 500 μE) for approximately 2 weeks to 4 weeks. Then, the chemical treatment was performed (Routine 1 kg granule, the plants were treated every 5 days with the chemical in an amount of 0.5 g/individual at one time, hence treated three times in total) to examine the flowering. As a result, the flowering was observed again in the chemically treated individuals in any of the tested lines (FIGS. 24, 25).

This Example demonstrated that the flowering of the lines produced according to the present invention was stably/reproducibly inducible.

### Example 11

### Expression Induction Test on Gene (12) Ortholog in Corn

As a result of the BLAST search in the genome sequence information database of corn (Zea Mays) (Maize GDB; http://www.maizegdb.org/) using the amino acid sequence of the rice gene (12) as a query, an orthologous gene (GRMZM2G169947; http://www.maizegdb.org/cgi-bin/displaygenemodelrecord.cgi?id=GRMZM2G169947) of the gene (12) was found. Whether the expression of the gene (12) ortholog in corn was induced by the plant activator treatment was tested.

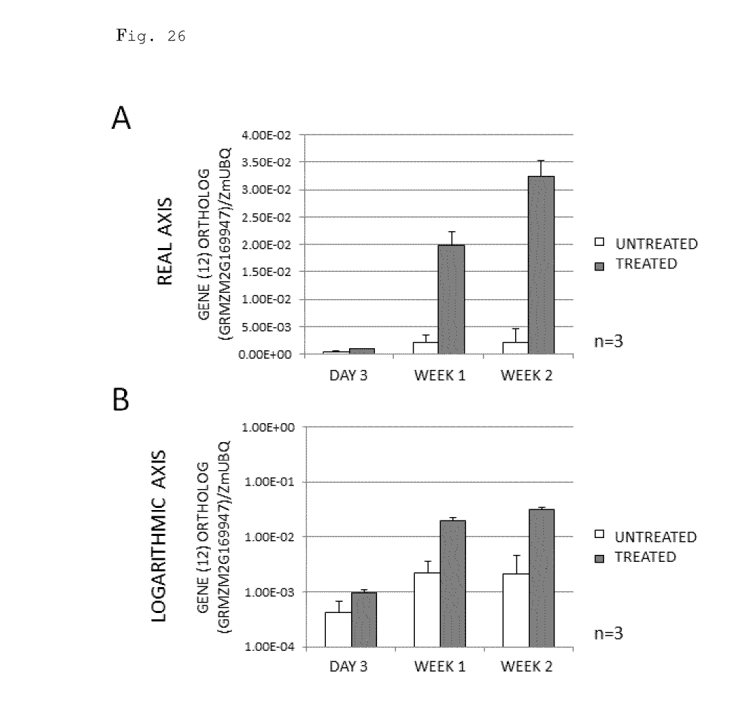

Corn (cultivar: Mi29) was grown in a growth chamber (long-day conditions: 14.5 hours of the light period: 9.5 hours of the dark period, temperature setting: 28° C. during the light period: 25° C. during the dark period, illumination: a metal-halide lamp of 500 μE). The plants on Week 2 after the seeding were subjected to the spray treatment with Routine 1 kg granule (Bayer CropScience AG) (0.4 g/individual). On Day 3, Week 1, and Week 2 after the chemical treatment, leaves were collected from each plant for the quantitative RT-PCR analysis. The quantitative RT-PCR analysis was conducted according to the method described in Example 4. Table 4 shows the sequence information on the primers. As a result of the quantitative RT-PCR analysis, a significant increase (10-fold or more) at the expression level of the gene (12) ortholog in corn was also observed in the chemically treated samples, and the induction by the plant activator was confirmed (FIG. 26).

This Example demonstrated that the present invention was applicable to a Poaceae crop corn, and that the flowering induction DNA cassette of the present invention (FIG. 2) was applicable to corn.

### Example 12

### Production of Transgenic Corn Plants

(1) Preparation of Corn Immature Embryos

In a greenhouse, one individual of corn plants was grown in one pot. The day-time temperature was maintained at 30 to 35° C., and the night-time temperature was maintained at 20 to 25° C. The light conditions were: the quantity of light was 60,000 1× or more, and the light period was 12 hours or more. Female ears containing immature embryos at a normal developmental stage were collected between Days 8 and 15 after the pollination. A husk was peeled from each female ear, and approximately the half of an upper portion of a grain was cut with a scalpel blade. The scalpel blade was inserted into the remaining grain, and the immature embryo was taken out on a tip of the scalpel. An immature embryo having a size of 1.0 to 1.2 mm is suitable for the transformation. The embryos were immersed in 2 ml of an LS-inf medium at room temperature, the medium having been put in a 2-ml tube. Thus, approximately 200 embryos were collected. The embryos are preferably collected within 1 hour. The tube with the embryos put therein was stirred at 2, 700 r.p.m. at room temperature for 5 seconds. Then, the LS-inf medium was removed. Two ml of a fresh LS-inf medium was added, followed by stirring in the same manner.

(2) Pretreatment and Centrifugation

The tube with the immature embryos put therein was incubated as a whole in a thermostatic chamber at 46° C. for 3 minutes. The tube with the immature embryos put therein was cooled as a whole on ice for 1 minute. The LS-inf medium was removed, and 2 ml of a fresh LS-inf medium was added. the tube with the immature embryos put therein was centrifuged at 20,000 g at 4° C. for 10 minutes.

(3) Preparation of Agrobacterium

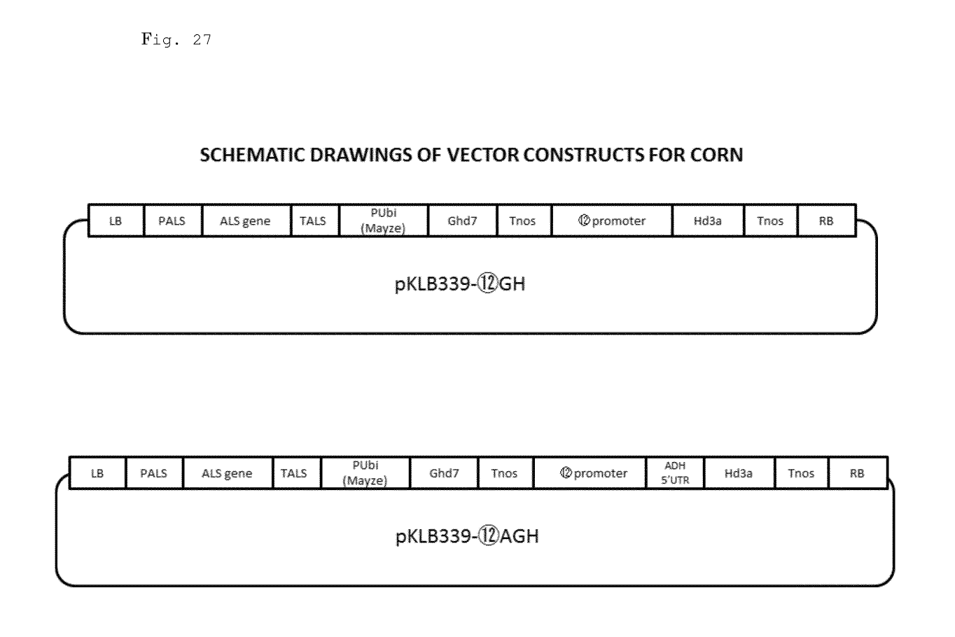

On a YP medium supplemented with necessary selection chemicals, Agrobacterium (LBA4404) was cultured in the dark at 28° C. for 2 days. The bacteria were collected with a loop and suspended in 1 ml of an LS-inf-AS medium at a cell density of 1×10⁹ cfu/ml (0D=1.0 at 660 nm). Note that FIG. 27 shows a schematic drawing of vector constructs for corn introduced into Agrobacterium. To increase the efficiency of introducing the exogenous gene into the plant genome, it is preferable to further introduce a helper plasmid (Japanese Patent No. 4534034) into Agrobacterium.

(4) Inoculation and Co-Culturing

After the centrifugation, the medium was removed from the tube, and 1 ml of the Agrobacterium suspension was added thereto. The tube was suspended at 2700 r.p.m. for 30 seconds. The resultant was left standing at room temperature for 5 minutes. The suspension of the immature embryos and Agrobacterium was transferred to an empty Petri dish (60×15 mm). From the suspension, 0.7 ml of the liquid portion was removed and discarded. The immature embryos were transferred to an LS-AS solid medium in such a manner that the scutella faced upward, and the Petri dish was sealed with a paraffin film. Approximately 100 of the immature embryos were left standing on one Petri dish. The co-culturing was performed in the dark at 25° C. for around 14 days.

(5) Selection of Transformed Calli

The immature embryos were transferred to an LSD1.5A medium, and the Petri dish was sealed with a paraffin film. Approximately 25 of the embryos were placed on one Petri dish. The culturing was performed in the dark at 25° C. for 10 days (first selection). The immature embryos were transferred to an LSD1.5B medium, and the Petri dish was sealed with a surgical tape. Approximately 25 of embryos were placed on one Petri dish. The culturing was performed in the dark at 25° C. for 10 days (second selection).

(6) Re-Differentiation of Transgenic Plants

Further grown type I calli were transferred to an LSZ medium, and the Petri dish was sealed with a paraffin film. Approximately 25 of the calli were placed on one Petri dish. The dish was irradiated with continuous light of 5,0001× at 25° C. for 14 days or more. Re-differentiated shoots were transferred to a tube containing an LSF medium, and the tube was closed with a polypropylene cap. The tube was irradiated with continuous light of 5,000 1× at 25° C. for 14 days or more. Each plant was transferred to a pot with appropriate soil. The transgenic plants were grown in the above-described greenhouse for 3 to 4 months.

Note that the compositions of reagent stocks for culturing and media used in this Example were as follows.

<Compositions of Reagent Stocks for Culturing>

| | |
|---|---|
| [10 × LS major salts] | |
| KNO₃ | 19.0 g |
| NH₄NO₃ | 16.5 g |
| CaCl₂•2H₂O | 4.4 g |
| MgSO₄•7H₂O | 3.7 g |
| KH₂PO₄ | 1.7 g/1000 ml |
| [100 × FeEDTA] | |
| FeSO₄•7H₂O | 2.78 g |
| Na₂EDTA | 3.73 g/1000 ml |
| [100 × LS minor salts] | |
| MnSO₄•5H₂O | 2.23 g |
| ZnSO₄ | 1.06 g |
| H₃BO₄ | 620 mg |
| KI | 83 mg |
| Na₂MoO₄•2H2O | 25 mg |
| CuSO₄•5H₂O | 2.5 mg |
| CoCl₂•6H₂O | 2.5 mg/1000 ml |
| [100 × modified LS vitamins] | |
| myoinositol | 10 g |
| thiamine hydrochloride | 100 mg |
| pyridoxine hydrochloride | 50 mg |
| nicotinic acid | 50 mg/1000 ml |
| 100 mg/L 2,4-D | |
| 100 mg/L zeatin | |
| 100 mg/L IBA | |
| 100 mg/L 6BA | |
| 100 mM acetosyringone | |
| 100 mM X-gluc | |
| 50 mM Na₂HPO₄ | |
| 50 mM NaH₂PO₄ | |

<Medium Compositions>

| | |
|---|---|
| [YP plate (for Agrobacterium)] | |
| yeast extract | 5 g |
| peptone | 10 g |
| NaCl | 5 g/1000 ml |
| pH | 6.8 |
| agar | 15 g |
| pour into a Petri dish after | |
| autoclaving | |
| [LS-inf medium (for preparation | |
| of immature embryos)] | |
| 10 × LS major salts | 100 ml |
| 100 × FeEDTA | 10 ml |
| 100 × LS minor salts | 10 ml |
| 100 × modified LS vitamins | 10 ml |
| 100 mg/L 2,4-D | 15 ml |
| sucrose | 68.46 g |
| glucose | 36.04 g |
| casamino acid | 1.0 g/1000 ml |
| pH | 5.2 |
| sterilized with a 0.22-μM | |
| cellulose-acetate filter | |
| [LS-inf-AS medium | |
| (for infection)] | |
| LS-inf medium | 1 ml |
| 100 mM acetosyringone | 1 μl |
| [LS-AS medium | |
| (for co-culturing)] | |
| 10 × LS major salts | 100 ml |
| 100 × FeEDTA | 10 ml |
| 100 × LS minor salts | 10 ml |
| 100 × modified LS vitamins | 10 ml |
| 100 mg/L 2,4-D | 15 ml |
| 100 mM CuSO₄ | 0.05 ml |
| sucrose | 20 g |
| glucose | 10 g |
| proline | 0.7 g |
| MES | 0.5 g/1000 ml |
| pH | 5.8 |
| agarose | 8 g |
| autoclave | |
| 100 mM acetosyringone | 1 ml |
| 100 mM AgNO3 | 0.05 ml |
| pour into a Petri dish | |
| [LSD 1.5 A medium] | |
| (for first selection of | |
| transformed cells) | |
| 10 × LS major salts | 100 ml |
| 100 × FeEDTA | 10 ml |
| 100 × LS minor salts | 10 ml |
| 100 × modified LS vitamins | 10 ml |
| MES | 0.5 g/1000 ml |
| pH | 5.8 |
| agar | 8 g |
| autoclave | |
| 250 g/L carbenicillin | 1 ml |
| 250 g/L cefotaxime | 0.4 ml |
| 100 mM AgNO₃ | 0.1 ml |
| 20 g/L phosphinothricin | 0.25 ml or |
| (bar selection) | |
| 50 g/L Hygromycin | 0.3 ml |
| (hpt selection) | |
| pour into a Petri dish | |
| [LSD 1.5 B medium] | |
| (for second and third selections | |
| of transformed cells) | |
| 10 × LS major salts | 100 ml |
| 100 × FeEDTA | 10 ml |
| 100 × LS minor salts | 10 ml |
| 100 × modified LS vitamins | 10 ml |
| MES | 0.5 g/1000 ml |
| pH | 5.8 |
| agar | 8 g |
| autoclave | |
| 250 g/L carbenicillin | 1 ml |
| 250 g/L cefotaxime | 0.4 ml |
| 100 mM AgNO₃ | 0.1 ml |
| 20 g/L phosphinothricin | 0.5 ml or |
| (bar selection) | |
| 50 g/L Hygromycin | 0.6 ml |
| (hpt selection) | |
| pour into a Petri dish | |
| [LSF medium (for root | |
| development)] | |
| 10 × LS major salts | 100 ml |
| 100 × FeEDTA | 10 ml |
| 100 × LS minor salts | 10 ml |
| 100 × modified LS vitamins | 10 ml |
| 100 mg/L IBA | 2 ml |
| sucrose | 15 g |
| MES | 0.5 g/1000 ml |
| pH | 5.8 |
| gellan gum | 3 g |
| pour into a tube, then autoclave | |
| [LSZ medium] | |
| (for re-differentiation of | |
| transformed cells) | |
| 10 × LS major salts | 100 ml |
| 100 × FeEDTA | 10 ml |
| 100 × LS minor salts | 10 ml |
| 100 × modified LS vitamins | 10 ml |
| 100 mg/L zeatin | 50 ml |
| 100 mM CuSO₄ | 0.1 ml |
| sucrose | 20 g |
| MES | 0.5 g/1000 ml |
| pH | 5.8 |
| agar | 8 g |
| autoclave | |
| 250 g/L carbenicillin | 1 ml |
| 250 g/L cefotaxime | 0.4 ml |
| 20 g/L phosphinothricin | 0.25 ml or |
| (bar selection) | |
| 50 g/L Hygromycin | 0.6 ml |
| (hpt selection) | |
| pour into a Petri dish | |
| [ELA medium] | |
| 10 × LS major salts | 100 ml |
| 100 × FeEDTA | 10 ml |
| 100 × LS minor salts | 10 ml |
| 100 mg/L 6BA | 5 ml |
| MES | 0.5 g/1000 ml |
| pH | 5.8 |
| agar | 8 g |
| autoclave | |
| Basta | 0.1 ml |
| (bar selection) | |
| 50 g/L Hygromycin | 2 ml |
| (hpt selection) | |
| pour into a Petri dish. | |

### Example 13

### Production of Transgenic Corn Plants

(1) Isolation of Promoter Region of Gene (12) Ortholog in Corn

In order to isolate a promoter region of the gene (12) ortholog in corn described in Example 11, PCR amplification was performed using the genomic DNA of corn (cultivar: Mi29) as a template, and a combination of a primer GRMZM2G169947_pro_Fw (5′-CGGGATCATTGTCGGCCCTTTAACCCCATT-3′/SEQ ID NO: 134) and a primer GRMZM2G169947_pro_Rv (5′-CGATCTCTCTCTCTCTCTCTCTCCACACAGCCCTCTCTGTCTCTAGATAC-3′/SEQ ID NO: 135), or a primer GRMZM2G169947_pro6.5_Fw (5′-CAGAAGGTTGTAACCAAGCAACTCTACTAG-3′/SEQ ID NO: 136) and a primer GRMZM2G169947_pro_Rv (5′-CGATCTCTCTCTCTCTCTCTCTCCACACAGCCCTCTCTGTCTCTAGATAC-3′/SEQ ID NO: 135). Two types of the promoter fragments (SEQ ID NOs: 133 and 137) having different sizes were each cloned in pCR 8/GW/TOPO (Invitrogen Corporation).

(2) Preparation of Transformation Vector Plasmid

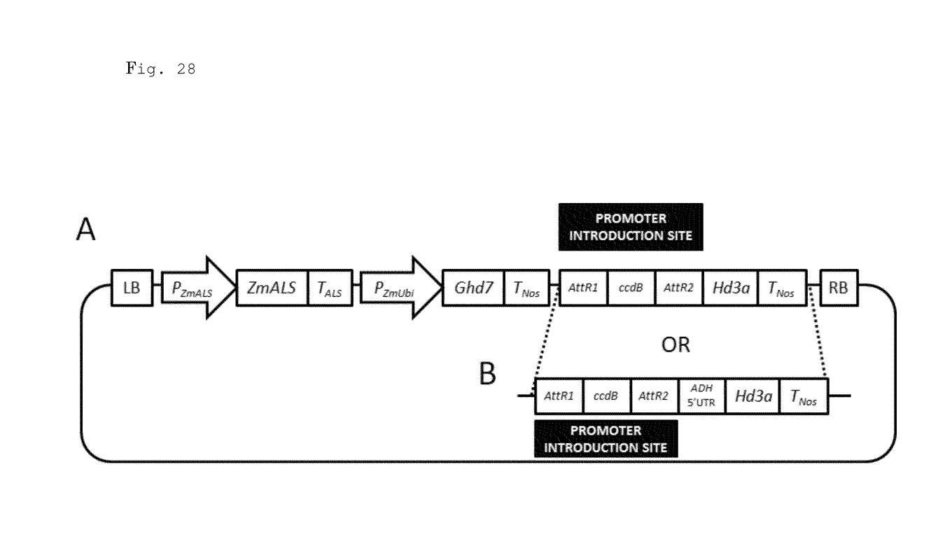

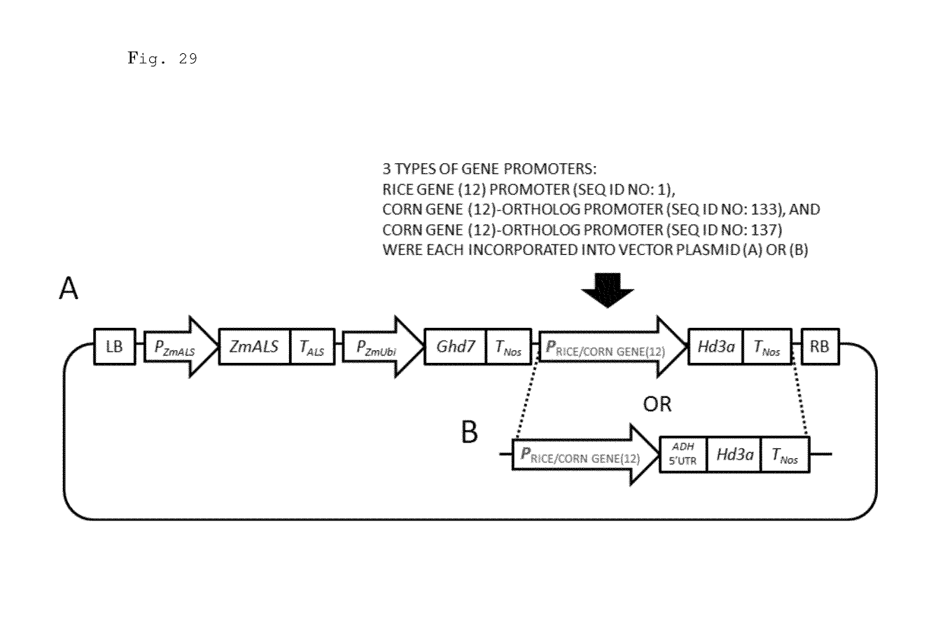

A vector plasmid used for corn transformants was prepared as follows. First, using each of the two flowering-time control plasmids pRiceFOX/Ubi:Ghd7/Gate:Hd3a (FIG. 2A, SEQ ID NO: 19) and pRiceFOX/Ubi:Ghd7/Gate:Adh5′UTR:Hd3a (FIG. 2B, SEQ ID NO: 20) as a template, PCR amplification was performed with a primer KLB525_UbiGhd7_fw_inf (5′-TACCGAGCTCGAATTCTGCAGCGTGACCCGGTCGTG-3′/SEQ ID NO: 138) and a primer KLB525_Tnos_rv2_inf (5′-AGTTTAAACTGAATTCCCGATCTAGTAACA-3′/SEQ ID NO: 139). Next, a site of a pKLB525 vector (Kumiai Chemical Industry Co., Ltd.) was treated with a restriction enzyme EcoRI. The two types of the fragments amplified by PCR above were cloned in the site using In-Fusion HD Cloning Kit (Takara). Thereby, binary vector plasmids pKLB525/Ubi:Ghd7/Gate:Hd3a (A in FIG. 28, SEQ ID NO: 140) and pKLB525/Ubi: Ghd7/Gate: Adh5′UTR: Hd3a (B in FIG. 28, SEQ ID NO: 141) were prepared which served as the nucleotides before the incorporation into the promoter. Then, each of the two corn-derived gene (12)-ortholog promoters (SEQ ID NOs: 133 and 137) and the rice-derived gene (12) promoter (SEQ ID NO: 1) was incorporated into promoter introduction sites of pKLB525/Ubi:Ghd7/Gate:Hd3a and pKLB525/Ubi:Ghd7/Gate:Adh5′UTR:Hd3a by the LR reaction. Thus, six types of transformation vector plasmids were prepared (FIG. 29).

(3) Production of Corn Transformants

According to the screening having been conducted in advance, a dent corn inbred line Mi29 was used as a starting material which was grown in Japan (Kyushu Okinawa Agricultural Research Center), suitable for plant tissue culture and also excellent in the ability as a parent of an F1 cultivar.

Mi29 was transformed by infecting immature embryos with Agrobacterium according to the method of Ishida et al. (Nature Protocol 2 (7): 1614-1621, 2007) except for chemicals used for selection. Unless otherwise stated, the culturing was performed using a sterilized Petri dish 9 cm in diameter basically with a MS solid medium. Immature embryos of Mi29 7 to 10 days after the fertilization were isolated and immersed in Agrobacterium (LBA4404 strain) having the transformation vector plasmid and a helper vector for increasing the transformation efficiency. Then, a high temperature treatment at 46° C. for 3 minutes and a centrifugation treatment at 4° C. at 20000 G for 10 minutes were performed. The resultant was cultured in a MS medium supplemented with 0.1 mM acetosyringone (LS-AS medium) at 25° C. in the dark for approximately 1 week. Several days later, calli were formed from the immature embryos, and then transferred to a medium supplemented with 0.5 μM bispyribac-sodium salt, 250 mg/l of carbenicillin and 100 mg/l of cefotaxime, followed by culturing at 25° C. in the dark for 2 weeks. The calli survived by exhibiting resistance to the bispyribac-sodium salt were further cultured in the same medium for 2 weeks. Subsequently, the calli were cultured in a MS medium supplemented with 5 mg/l of zeatin (LSZ medium) at 28° C. under continuous illumination for 2 weeks to 1 month to promote shoot formation. The calli having shoots formed were transferred to a MS medium supplemented with 0.2 mg/l of IBA (LSF medium) in a test bottle to promote root development. After approximately 2 weeks to 1 month, recombinants having sufficient root development in the test bottle were transferred to a plastic pot 9 cm in diameter filled with Kureha gardening soil.

From the plants in the test bottle or after the potting, leaves were cut to approximately 25 to 50 mg, and the DNA was extracted by the simple method. The gene introduced in the transformant was confirmed using this DNA as a template, and the following primers. For the Hd3a gene: GRMZM2G169947_pro_colonyfw2 (5′-CTGTGGACTGTAGATCTCCATATGTA-3′/SEQ ID NO: 142) and Hd3a/R (sacI) (5′-gagctcctagttgtagaccc-3′/SEQ ID NO: 79). For the Ghd7 gene: 3UBQMF2 (5′-tttagccctgccttcatacgct-3′/SEQ ID NO: 76) and 3Lhd4R1 (5′-CGTCGTTGCCGAAGAACTGG-3′/SEQ ID NO: 77).

### Example 14

### Expression Induction Test by Plant Activator Treatment on Corn Transformants

The potted transformants described in Example 13 were grown in a self-contained greenhouse under natural light supplemented with light of a fluorescent lamp for plant growth for 2 hours in every morning and evening. After approximately 2 weeks, the transformants were further transferred to deep Wagner pots.

Seven transformant individuals having the introduced gene obtained by using the rice gene (12) promoter (SEQ ID NO: 1) and the corn gene (12)-ortholog promoter (SEQ ID NO: 133) were used for the chemical induction test. A silicone plug was put in a discharge hole at the bottom of the Wagner pot to flood the pot, and 0.5 g of Routine 1 kg granule (Bayer CropScience AG) was applied to the liquid surface approximately 1 cm from the soil surface, followed by stirring to suspend and dissolve the chemical. After 24 hours, the silicone plug was pulled to discharge the water. Moreover, 5 hours before the chemical induction, leaf blades were sampled in advance as samples untreated with the chemical. Further, leaf blade samples were collected from the same individuals again one week after the chemical treatment, and subjected to RNA extraction.

The RNA extraction from the leaf blade samples, the cDNA synthesis, and the quantitative RT-PCR analysis were carried out by the methods described in Example 4. In addition, Table 4 shows the primer sequences used in the quantitative RT-PCR analysis.

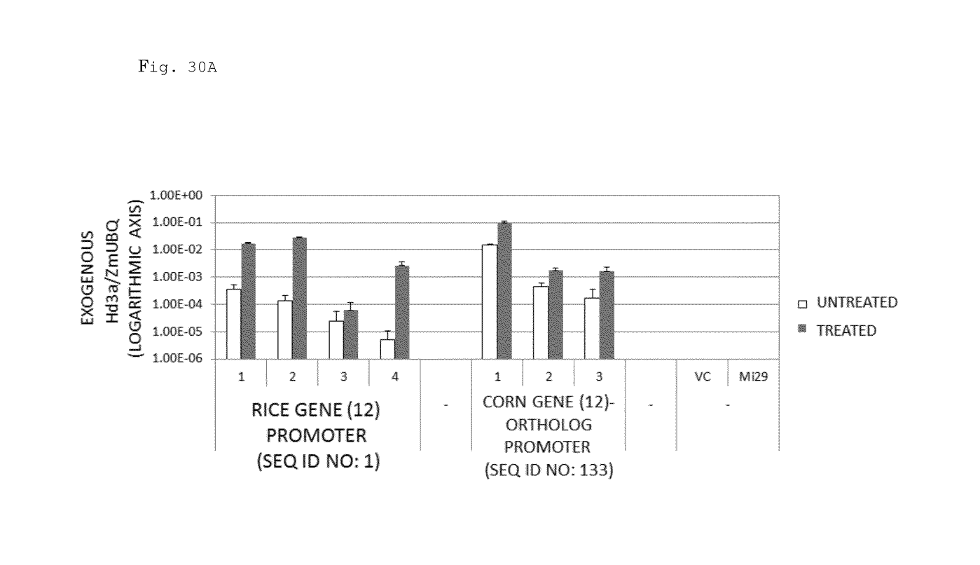

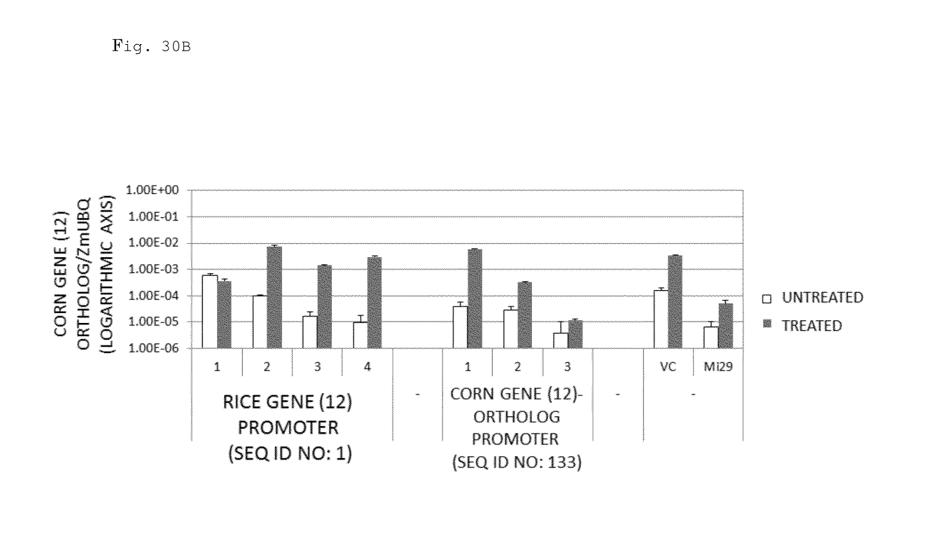

As a result of the expression analysis in the corn transformants, whichever the rice gene (12) promoter (SEQ ID NO: 1) and the corn gene (12)-ortholog promoter (SEQ ID NO: 133) were used, higher values at the expression level of exogenously introduced Hd3a in the leaves were exhibited in the treatment than in the untreatment (several ten-fold to several hundred-fold or more inductions were observed in lines having a large difference therebetween). The transcription inductions of the promoters by the chemical were confirmed also in corn (FIG. 30A). Moreover, in examining the endogenous expression of the corn gene (12) ortholog in each line, the expression level in the leaves was higher in the treatment than in the untreatment as described above. The effect of the chemical treatment was confirmed (FIG. 30B).

This Example demonstrated that the activity of the corn gene (12)-ortholog promoter exhibited a plant-activator induction, and that the rice gene (12) promoter functioned also in a Poaceae crop corn as in Examples for rice. Thus, it was demonstrated that the present invention was applicable to a Poaceae crop cultivar by using a promoter of a homologous gene of the gene (12).

### Example 15

### Expression Induction Test by Plant Activator Treatment on Rice Transformants Obtained by Using Corn Gene (12)-Ortholog Promoter

(1) Preparation of Transformation Binary Vectors

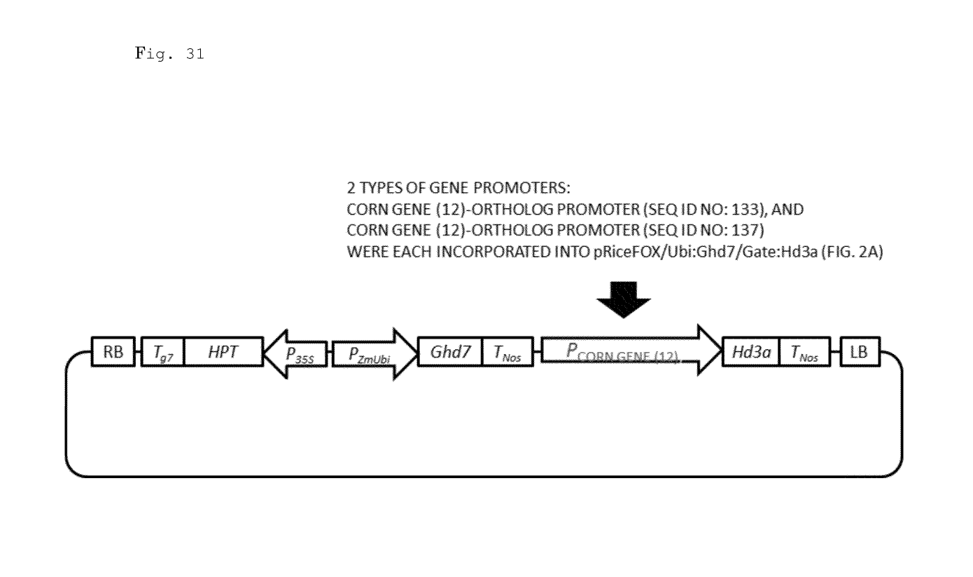

Each promoter fragment of the corn-derived gene (12)-ortholog promoters (SEQ ID NOs: 133 and 137) having different sizes was incorporated into the promoter introduction site of the flowering-time control plasmid (pRiceFOX/Ubi:Ghd7/Gate:Hd3a, FIG. 2A, SEQ ID NO: 19) described in Example 2 by the LR reaction. Thus, two types of transformation binary vectors were prepared (FIG. 31).

(2) Rice Transformation

To transform rice (cultivar: Nipponbare) with the two types of transformation vectors, the aforementioned method described in Example 1 was used. The genes introduced in the produced rice transformants were confirmed by the genomic PCR analysis using the following primers. For the Hd3a gene: GRMZM2G169947_pro_colonyfw2 (5′-CTGTGGACTGTAGATCTCCATATGTA-3′/SEQ ID NO: 142) and Hd3a/R (sacI) (5′-gagctcctagttgtagaccc-3′/SEQ ID NO: 79). For the Ghd7 gene: 3UBQMF2 (5′-tttagccctgccttcatacgct-3′/SEQ ID NO: 76) and 3Lhd4R1 (5′-CGTCGTTGCCGAAGAACTGG-3′/SEQ ID NO: 77). For the HPT gene: P35S1 (5′-TCCACTGACGTAAGGGATGA-3′/SEQ ID NO: 80) and Nos3 (5′-ATCAGCTCATCGAGAGCCT-3′/SEQ ID NO: 81).

(3) Expression Induction Analysis by Plant Activator Treatment

To handle the transformants in one line separately for the treatment/untreatment with the plant activator, tillers of each line were divided into two, transferred to a glass greenhouse, and grown under the flooded condition. Then, on Day 34 after the growth, the individuals for the treatment were subjected to the chemical spray treatment, and the induction test was started. As the chemical, Routine 1 kg granule (Bayer CropScience AG) was used, and the individuals were treated with the chemical in an amount of 0.5 g per individual. Leaf blades were collected from the untreated individuals and the treated individuals of each line 5 days after the treatment was started. The induction of the gene expression by the chemical was examined by the quantitative RT-PCR analysis. Meanwhile, for an undividable line which produced only one tiller, adopted was an analysis method in which a leaf was collected before the chemical treatment, and a leaf was collected from the same individual again after the treatment. The RNA extraction from the leaf blade samples, the cDNA synthesis, and the quantitative RT-PCR analysis were carried out by the methods described in Example 4. In addition, Table 4 shows the sequences of the primers and the Taq Man probes used in the quantitative RT-PCR analysis.

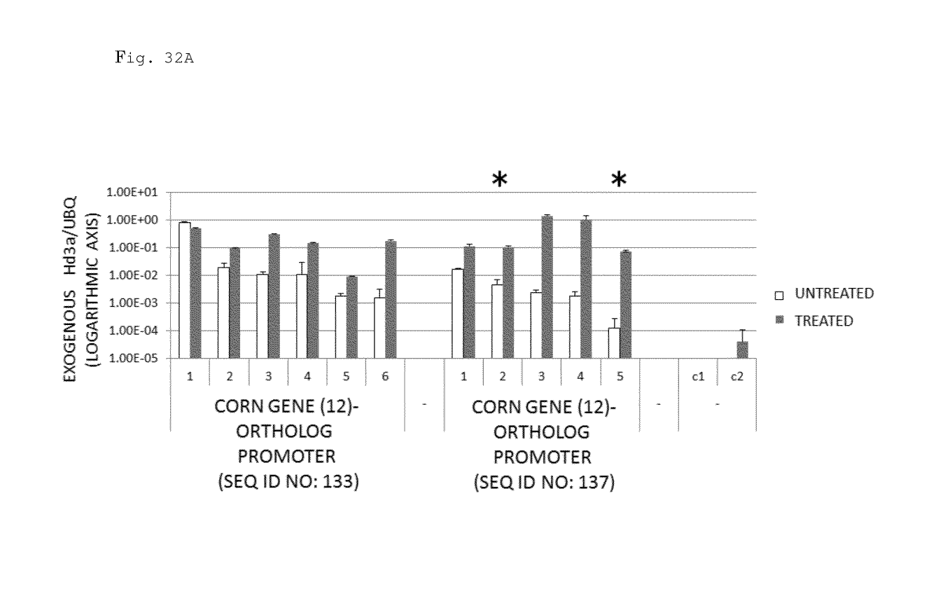

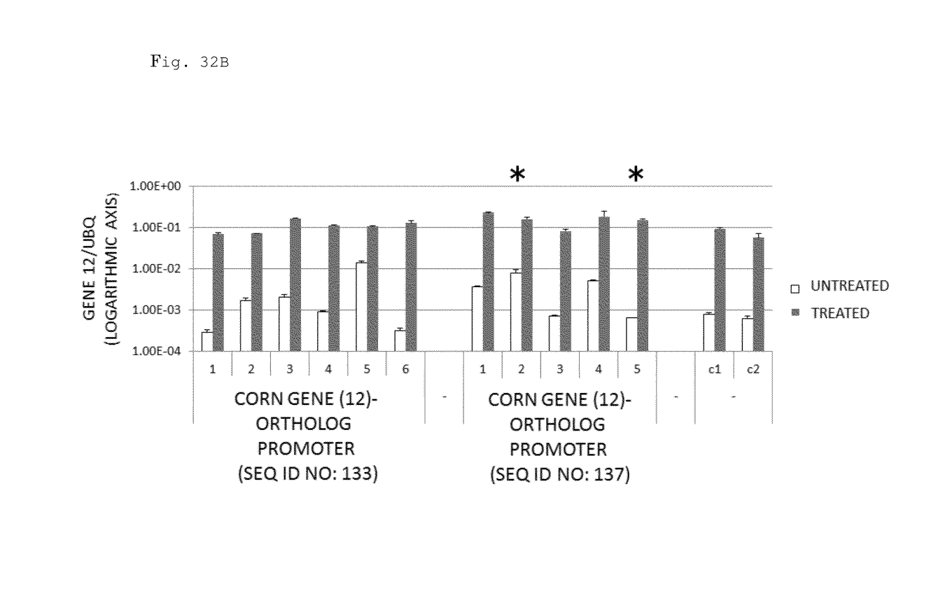

As a result of the expression analysis, whichever the two corn gene (12)-ortholog promoters (SEQ ID NOs: 133 and 137) having different sizes were used, the expression of exogenously introduced Hd3a was detected at a higher level in the treated leaves than the untreated leaves (several ten-fold to several hundred-fold or more inductions were observed in lines having a large difference therebetween). The induced expression by the chemical was confirmed (FIG. 32A). Moreover, in the analysis on the endogenous expression of the rice gene (12) as a positive control of the chemical induction also, a higher level of the induced expression was confirmed in the treated leaves (FIG. 32B).

This Example demonstrated that the promoter of the corn-derived gene (12) ortholog exhibited the plant-activator induction also in rice, and that ones derived not only rice but also corn were usable in the present invention. Thus, it was demonstrated that the present invention was applicable to a Poaceae crop cultivar by using a promoter of a homologous gene of the gene (12).

### Example 16

### Production of Transgenic Sugarcane Plants

(1) Production of Transformants

(a) Transformation Vector

As a transformation vector, the transformation plasmid comprising the gene (12) promoter described in Example 4 was used.

(b) Gene Introduction Method Using Agrobacterium

From a curly leaf at the head part of a sugarcane cultivar Saccharum spp. Q165 grown in a greenhouse for approximately around 3 months, 1-cm white curly leaf pieces were aseptically isolated, placed on a callus induction N6D medium, and subcultured at 28° C. under a dark condition for approximately 4 months. Thereby, yellow callus masses were obtained. In this event, the tissue pieces were transferred to a fresh medium once every 3 weeks. The obtained calli were trans formed by the Agrobacterium method with the vector described in Section (a). In the transformation, an Agrobacterium EHA105 strain was utilized. The calli treated with Agrobacterium were placed on an N6D medium and co-cultured at 28° C. under a dark condition for 3 days. Then, Agrobacterium was eliminated with sterile water and a carbenicillin solution, and the resultant was placed on a selection N6D medium supplemented with 50 mg/l of hygromycin and 500 mg/l of carbenicillin. The callus selection was performed at 28° C. under a dark condition for 1 to 2 months. During this period, the calli were transferred to a fresh medium once every 2 weeks. The obtained hygromycin resistant calli were placed on a re-differentiation medium N6RE supplemented with 50 mg/l of hygromycin and 500 mg/l of carbenicillin, and cultured under conditions of 28° C. and 16L/8D (16 hours of the light period, 8 hours of the dark period) for approximately 1 month. In this event, the tissue pieces were transferred to a fresh medium once every 2 weeks. The obtained re-differentiated plants were transferred to a hormone-free MS medium supplemented with 50 mg/l of hygromycin and 500 mg/l, and cultured under conditions of 28° C., 16L/8D (16 hours of the light period, 8 hours of the dark period), and 50 μmol/m²/s for approximately 1 month. In this event, the tissue pieces were transferred to a fresh medium once every 2 weeks. The re-differentiated individuals were used as recombinants for the experiment.

Note that the compositions of the media were as follows.

[N6D Medium]

N6 medium: 3.95 g/l, sucrose: 30 g/l, plant medium agar: 0.9 g/l, N6 vitamins (*): 1 ml/l, micro+α(**): 1 ml/1, 2, 4-D: 5 mg/l, pH: 5.8

[N6RE Medium]

N6 medium: 3.95 g/l, sucrose: 30 g/l, plant medium agar: 0.9 g/l, N6 vitamins(*): 1 ml/l, micro+α(**): 1 ml/l, BAP: 1 mg/l, casamino acid: 500 mg/l, pH: 5.8

[MS Medium]

N6 medium: 3.95 g/l, sucrose: 30 g/l, gellan gum: 2 g/l, N6 vitamins (*): 1 ml/l, micro+α(**): 1 ml/l, pH: 5.8

[ (*) N6 Vitamins Stock Solution]

glycine: 2 mg/l, thiamine hydrochloride: 1 mg/l, pyridoxine hydrochloride: 0.5 mg/l, nicotinic acid: 0.5 mg/l, myo-inositol: 100 mg/l

[ (**) Micro+α Stock Solution]

CuSO₄.5H₂O: 0.025 mg/ml, CoCl₂.6H₂O: 0.025 mg/ml, Na₂MoO₂.2H₂O: 0.25 mg/ml
(2) Oryzemate Treatment Test

(a) Growth Conditions

The plants such as 12tH recombinants were grown in a growth chamber (Koitotron, Koito Electric Industries, Ltd.) set at a temperature of 28° C., 16L/8D (16 hours of the light period, 8 hours of the dark period), 210 μmol/m2/s, and a humidity of 55%. The pots were supplied with water from the top.

(b) Oryzemate Treatment Method

When 12 days elapsed after the potting, an Oryzemate treatment was performed on the plants. In a treated plot with Oryzemate, Oryzemate granule (probenazole 8%, manufactured by Meiji Seika Pharma Co., Ltd.) suspended at 9 g/L was sprayed in an amount of 100 ml per individual on Day 1 (first time), Day 4 (second time), and Day 10 (third time) from the top of the pot. In an untreated plot with Oryzemate, the pot was sprayed from the top with water in the same amount as that in the treated plot.

(c) Sampling

On Day 4 (second time) and Day 10 (third time) of the Oryzemate treatment, mature leaves were obtained from the plants before the Oryzemate treatment.

(3) Expression Analysis

(a) RNA Extraction, Reverse Transcription

Using RNeasy Plant Mini Kit (manufactured by QIAGEN), the total RNAs of the plant mature leaves obtained above were extracted and purified. The template cDNAs were synthesized using PrimeScript RT reagent Kit (manufactured by Takara Bio Inc.).

(b) Expression Analysis

The RNA expression analysis was conducted by the real-time PCR using ABI 7500 Real Time PCR System (manufactured by Applied Biosystems Inc.).

The amplification products of actin and the Hd3a gene were quantified using the SYBR Green method (SYBR premix Ex Taq manufactured by Takara Bio Inc.) (primers for actin: T06F CA000593_F, T06R CA000593_R, primers for Hd3a: AgqOsH3-F, AgqOsH3-R).

The amplification product of the Ghd7 gene was quantified using the TaqMan method (Premix Ex Taq manufactured by Takara Bio Inc.) (primers for Ghd7: OsBrqtG7-F, OsBrqtG7-R, TaqMan probe: OsBrqtG7-P).

### Example 17

### Production of Transgenic Sugarcane Plants

(1-1) Materials
Sugarcane head part (the uppermost node portion, cultivar: Q165)
12AGH vector

Note that the 12AGH vector is the transformation vector obtained by introducing the rice gene (12) promoter (SEQ ID NO: 1) into the promoter introduction site of the flowering-time control plasmid (pRiceFOX/Ubi:Ghd7/Gate:Adh5′UTR:Hd3a, FIG. 2B).

(1-2) Callus Preparation

First, in a clean bench, the exodermis of the head part was peeled, and the resulting surface was sterilized with 70% ethanol. Then, the epidermis was peeled to have a diameter as thick as approximately 8 mm, and the resulting surface was again sterilized with 70% ethanol. The epidermis was further peeled to have a diameter as thick as approximately 5 mm. Subsequently, the upper portion near the growing point was cut to sections of approximately 5 mm. Seven such sections were placed on a callus induction medium in one Petri dish, and cultured at 28° C. under dark for 3 to 4 months. During this period, subculturing was performed every one month (callus induction medium: 1 L of N6, sucrose: 30 g, plant agar: 9 g, MS vitamins: 1 ml, thiamine hydrochloride: 1 mg, micro+α: 1 ml, 2-4, D: 5 mg, coconut water: 50 ml, Petri dish: 50 ml, pH: 5.8).

(1-3) Preparation of Agrobacterium for infection

The 12AGH vector was introduced into Agrobacterium EHA105 by electroporation, and the resultant was cultured in an LB agar medium supplemented with hygromycin (50 mg/L). The obtained single colony was cultured in an LB liquid medium supplemented with hygromycin (50 mg/L), and suspended in an 80% glycerol solution. This served as a stock solution.

(1-4) Preparation of Bacterial Solution for Infection and Co-Culture Medium

The stock solution was cultured overnight with an LB liquid medium supplemented with hygromycin (50 mg/L). The bacteria were collected and suspended in an N6 liquid medium. To the suspended Agrobacterium solution, 20 mg/L of acetosyringone was added, and the resultant was diluted with an N6 liquid medium. Thus, a bacterial solution for infection was prepared. A co-culture medium used was prepared by placing three sheets of filter paper (φ9 mm) into a deep Petri dish, which was then wetted with 5 mL of a co-culture liquid medium (co-culture liquid medium: 1 L of N6, sucrose: 30 g, MS vitamins: 1 ml, thiamine hydrochloride: 1 mg, micro+α: 1 ml, 2-4, D: 5 mg, acetosyringone: 20 mg, Petri dish: 9 ml, pH: 5.8).

(1-5) Infection and Co-Culturing

The callus prepared in (1-2) was immersed in the bacterial solution for infection prepared in (1-4) for the infection. After the immersion for approximately 10 minutes, the bacterial solution for infection was sucked well for the removal. The resultant was placed on a co-culture medium and cultured at 28° C. under dark for 4 days (co-culture medium: 1 L of N6, sucrose: 30 g, plant agar: 9 g, MS vitamins: 1 ml, thiamine hydrochloride: 1 mg, micro+α: 1 ml, 2-4, D: 5 mg, acetosyringone: 20 mg, Petri dish: 9 ml, pH: 5.8).

(1-6) Selection Culturing

After the 4-day co-culturing, the resultant was placed on a selection medium (selection medium: 1 L of N6, sucrose: 30 g, plant agar: 9 g, MS vitamins: 1 ml, thiamine hydrochloride: 1 mg, micro+α: 1 ml, 2-4, D:5 mg, hygromycin: 50 ml, carbenicillin: 500 mgl, Petri dish: 50 ml, pH: 5.8). The culturing was performed at 28° C. under dark for 2 to 3 months, followed by subculturing every one month.

(1-7) Re-Differentiation Culturing

The callus, yellowish and somewhat hard callus, on the selection medium was divided to pieces larger than 4 mm and transferred onto a re-differentiation medium (re-differentiation medium: 1 L of N6, sucrose: 30 g, plant agar: 9 g, MS vitamins: 1 ml, thiamine hydrochloride: 1 mg, micro+α: 1 ml, BAP: 1 mg, casamino acid: 500 mg, Petri dish: 50 ml, pH: 5.8). The culturing was performed under conditions of 28° C., 16 hours of the day length, and 50 μmol/m2/s, for 1 month.

(1-8) Culturing for Root Development

After a shoot grew to 2 to 3 cm or more during the re-differentiation induction, the shoot was transferred to a root development medium (root development medium: 1 L of N6, gellan gum: 2 g, MS vitamins: 1 ml, micro+α: 1 ml, 100 mL/pot, pH: 5.8). The culturing was performed under conditions of 28° C., 16 hours of the day length, and 50 μmol/m2/s, for 1 month.

(1-9) Acclimatization

The individual rooted to a total length of 5 cm in the root development medium was acclimatized. The plant was taken out from the culture pot, and the medium attached to the roots was removed. Then, the plant was transferred to a cell tray with vermiculite. After water was supplied thereto, the cell tray was put into a container so as to prevent water stress, all of which was covered with a plastic. The space between the plastic and the container was increased every one week, so that the acclimatization was completed in 1 month (growth chamber conditions: 28° C., 16 hours of the day length, 250 μmol/m2/s, a humidity of 60%).

(1-10) Gene Introduction Confirmation by PCR

The gene introductions were confirmed by PCR using the following primers for introducing the target genes. Those from which the introductions were confirmed were used as gene-introduced lines.

### Primers Used:

HPT: Nos3 (SEQ ID NO: 81, P35S1 (SEQ ID NO: 80) Ubi:Ghd7: 3UBQMF2 (SEQ ID NO: 76), 3Lhd4R1 (SEQ ID NO: 77) (12) promoter Hd3a: (12) Fw (SEQ ID NO: 83), Hd3a/R (Sac) (SEQ ID NO: 79)

(1-11) Result of Recombinant Production

By the above-described method, 40 individuals of the re-differentiated lines were obtained. Nevertheless, when the HPT gene introduction was confirmed by the PCR analysis using the primers for HPT as described in (1-10), it was confirmed that there were 18 lines in which this gene was introduced (hpt-introduced lines).

These 18 hpt-introduced lines were analyzed by PCR using the Ubi:Ghd7 primers, the (12) promoter Hd3a primers, and the Hd3a cDNA primers. As a result, it was confirmed that lines in which each gene was introduced were obtained as follows.

Lines from which the introductions of (12) promoter Hd3a, Hd3a cDNA, and Ubi:Ghd7 were confirmed: ten lines (Nos. 3, 5, 7, 10, 11, 12, 13, 14, 16, 18)

Line from which the introductions of (12) promoter Hd3a and Hd3a cDNA were confirmed: one line (No. 17).

### Example 18

### Expression Induction Test by Plant Activator Treatment on Sugarcane Transformants Obtained by Using Rice-Derived Gene (12) Promoter

(2-1) Materials
12AGH recombinants
Control lines (controls)

(2-2) Preparation of Induction Test Lines

As to the 12AGH lines, two individuals from each line having been confirmed that the genes were introduced were planted in one pot. Two such pots were used for each of a treated plot and an untreated plot.

As to the control lines (controls), a sugarcane axillary bud (stem) of each line was cut to 5 cm in length and planted in a cell tray with vermiculite to secure the plants 3 weeks before potting. In the same manner as above, two individuals were planted in one pot, and two such pots were used for each of the treated plot and the untreated plot (control lines: 12GH, Q165 (wild type)). Note that 12GH is a one-event line of sugarcane transformants produced by using the transformation vector obtained by introducing the promoter of the rice gene (12) (SEQ ID NO: 1) into the promoter introduction site of the flowering-time control plasmid (pRiceFOX/Ubi:Ghd7/Gate:Hd3a, FIG. 2A).

(2-3) Potting of Induction Test Lines

After the 12AGH-introduced lines and the control lines grew to approximately 20 cm, two individuals were planted in one pot, and two such pots were transferred (Bonsoru No. 2, Sumitomo Chemical Co., Ltd. in a plant pot R18 (diameter: 18 cm), GUNZE Ltd.). The plants were grown under conditions of 28° C., 16 hours of the day length, 250 μmol/m2/s, and a humidity of 60%.

(2-4) Induction Treatment and Sample Acquisition

After 2 weeks or more elapsed from the potting, the chemical treatment was started. Moreover, the day when the treatment was started was set as Day 1, and the Oryzemate treatment was performed on the treated plot on Days 1, 6, and 10. Note that, in the treatment, Oryzemate granule (probenazole 8%, manufactured by Meiji Seika Pharma Co., Ltd.) suspended at 9 g/L was sprayed in an amount of 100 ml per individual from the top of the pot. In the untreated plot, the pot was sprayed from the top with water in the same amount as that in the treated plot. Further, in both of the treated plot and the untreated plot, samples were obtained on Days 1, 6, 10, and 16, and 50 mg of the sample were obtained from two leaves at the tip of each plant immediately before each treatment with or without Oryzemate. The sample was frozen with liquid nitrogen and stored at −80° C.

(2-5) RNA Acquisition

The frozen sample was ground into a powder form using liquid nitrogen and a mortar. From the ground sample, the RNA was obtained using Rneasy Plant Mini Kit (QIAGEN). Regarding the DNase I treatment, the treatment was performed on a column using RNase-Free DNase Set (QIAGEN). The concentration was measured using NanoDrop 2000 (Thermo Scientific) and Bioanalyzer 2100 (RNA6000 kit, Agilent), and the amount used for the reverse transcription was determined.

(2-6) Reverse Transcription

The reverse transcription was performed using PrimeScripr RT reagent Kit (Takara).

(2-7) Ghd7 Expression Analysis

The Ghd7 expression analysis was conducted on the sample before the induction treatment. Each sample was analyzed in three replications using Premix Ex Taq (Takara) under the following conditions.

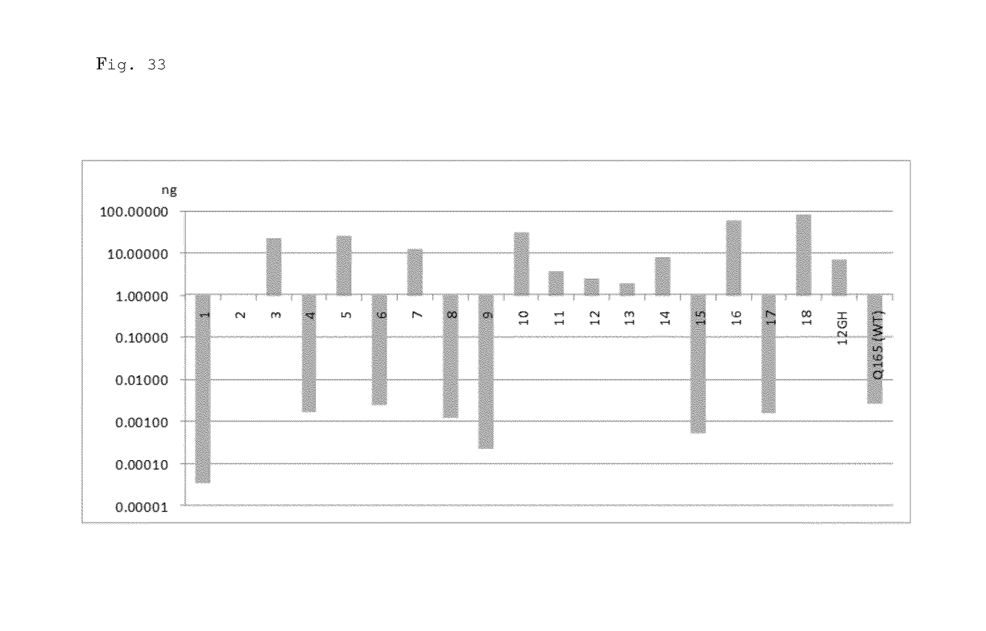

As samples for standard curve, 12GH-vector diluted lines (10⁷ to 10²) were used. OsBrqtG7-F and OsBrqtG7-R were used as a primer set, and OsBrqtG7-P was used as the Taq Man probe. Moreover, a reaction mixture containing these was heated at 95° C. for 30 seconds, and then reaction cycles each consisting of 95° C. for 5 seconds and 60° C. for seconds were repeated 4 times. FIG. 33 shows the obtained result.

(2-8) Result of Ghd7 expression analysis

The Ghd7 expression analysis as described in (2-7) was conducted on the 18 lines from which the introduction of the HPT gene was confirmed in (1-11). As a result, a high level of the Ghd7 gene expression was observed in the hpt-introduced lines Nos. 3, 5, 7, 10, 16, and 18 as shown in FIG. 33.

(2-9) Hd3a Expression Analysis

The Hd3a expression analysis was conducted on the samples subjected to the induction treatment. Each sample in both of the treated plot and the untreated plot was analyzed in three replications using SYBR premix Ex Taq (Takara) under the following conditions. The level of the Hd3a gene expressed in each sample was analyzed. Note that the lines were analyzed in the order of having a high level of the Ghd7 expression.

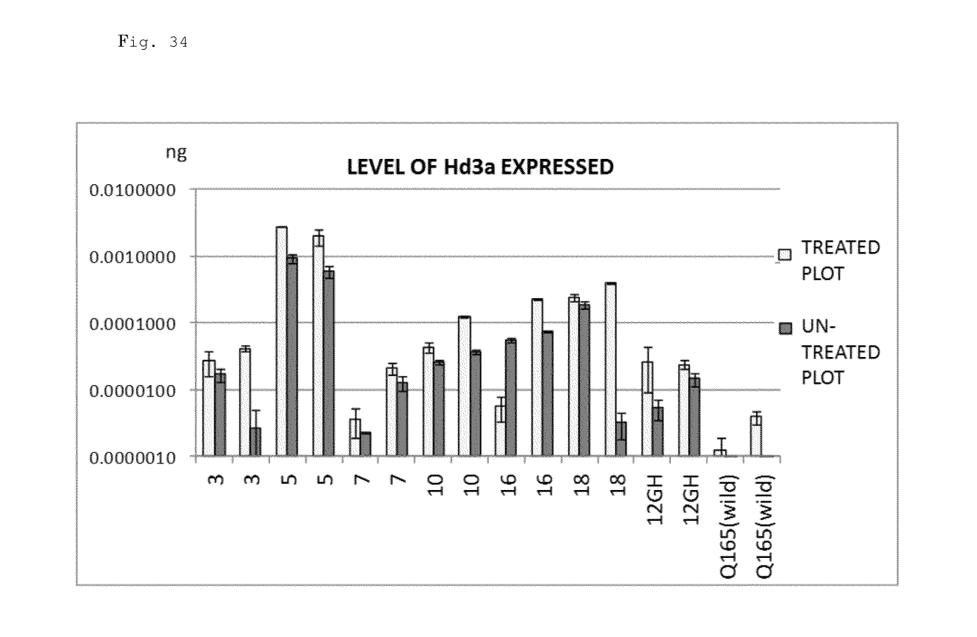

The 12GH-vector diluted lines (10⁷ to 10²) were used as samples for Hd3a standard curve, and AgqOsH3-F and AgqOsH3-R were used as an Hd3a primer set. Moreover, sugarcane Q165gDNA, actin-amplified sample diluted lines (10⁷ to 10²) were used as samples for actin standard curve, and ScActinT06F and ScActinT06R were used as an actin primer set. Further, a reaction mixture containing these was heated at 95° C. for 30 seconds, and then reaction cycles each consisting of 95° C. for 5 seconds and 60° C. for 34 seconds were repeated 40 times. Subsequently, the resultant was further subjected to 95° C. for 15 seconds and 60° C. for 1 minute, followed by heating at 95° C. for 15 seconds. FIG. 34 shows the obtained result.

(2-10) Result of Hd3a Expression Analysis

The lines (Nos. 3, 5, 7, 10, 16, and 18) having a high level of the Ghd7 expression revealed in (2-8) were examined as described in (2-9) for the expression level of the Hd3a gene exogenously introduced in such a manner that Hd3a was ligated to the rice gene (12) promoter. As a result, the expression of the Hd3a gene in sugarcane was detected as shown in FIG. 34. Moreover, higher expression values of the exogenous Hd3a gene were detected in the treatment than in the untreatment. The chemical induction by the plant activator treatment was confirmed also in sugarcane.

### Example 19

### Example 2 of Applying Flowering-Time Control DNA Cassette to Feed Rice Cultivar Kitaaoba

To transform the feed rice cultivar Kitaaoba, the flowering-time control plasmid (pRiceFOX//Ubi:Ghd7/Gate:Adh5′UTR:Hd3a, FIG. 2B) was used in which the rice gene (12) promoter (SEQ ID NO: 1) was introduced. The transformants (T0 generation) thus produced were subjected to the flowering induction test by the plant activator treatment. Kitaaoba was transformed according to the method described in Example 1.

Replicate individuals for the treatment and the untreatment with the plant activator were prepared by dividing tillers of each line of the transformants, and grown in a growth chamber (long-day conditions: 14.5 hours of the light period: 9.5 hours of the dark period, temperature setting: 28° C. during the light period: 25° C. during the dark period, illumination: a metal-halide lamp of 500 μE). On Day 37 thereafter, the plant activator spray treatment was started on the individuals for the treatment. As the chemical, Routine 1 kg granule (Bayer CropScience AG) was used, and the plants were treated every 5 days with the chemical in an amount of 0.5 g/individual at one time, hence treated three times in total. On Day 3 and Week 2 after the chemical treatment was started, leaf blades were collected from each line and subjected to the quantitative RT-PCR analysis.

The RNA extraction from the leaf blade samples, the cDNA synthesis, and the quantitative RT-PCR analysis were carried out by the methods described in Example 4. In addition, Table 4 shows the sequences of the primers and the Taq Man probes used in the quantitative RT-PCR analysis.

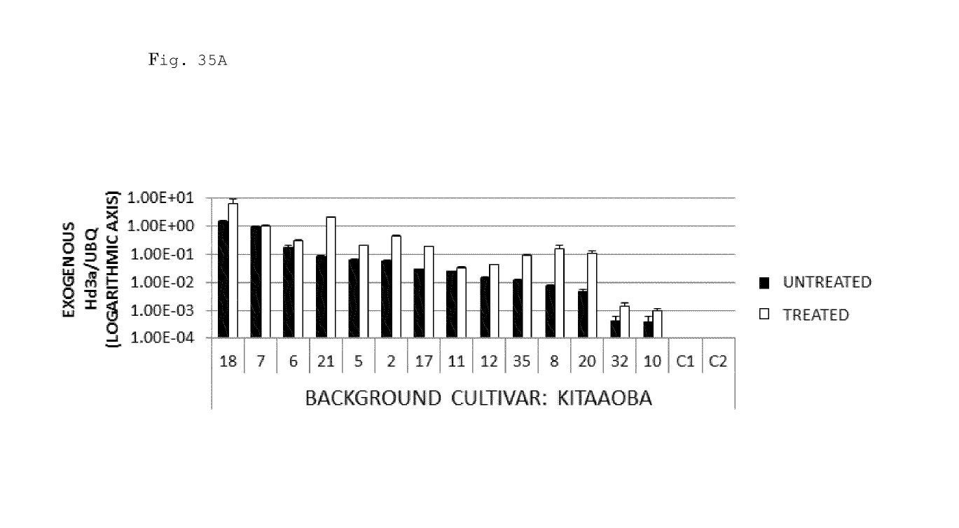

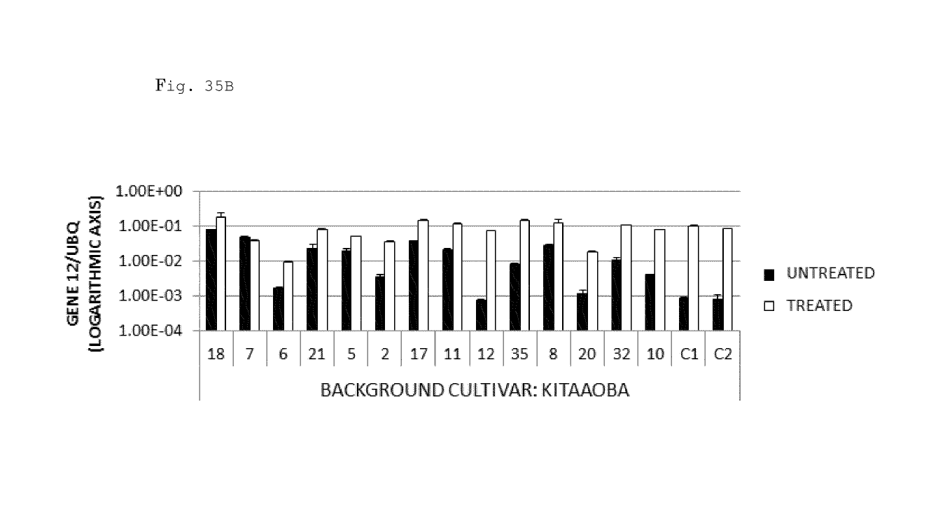

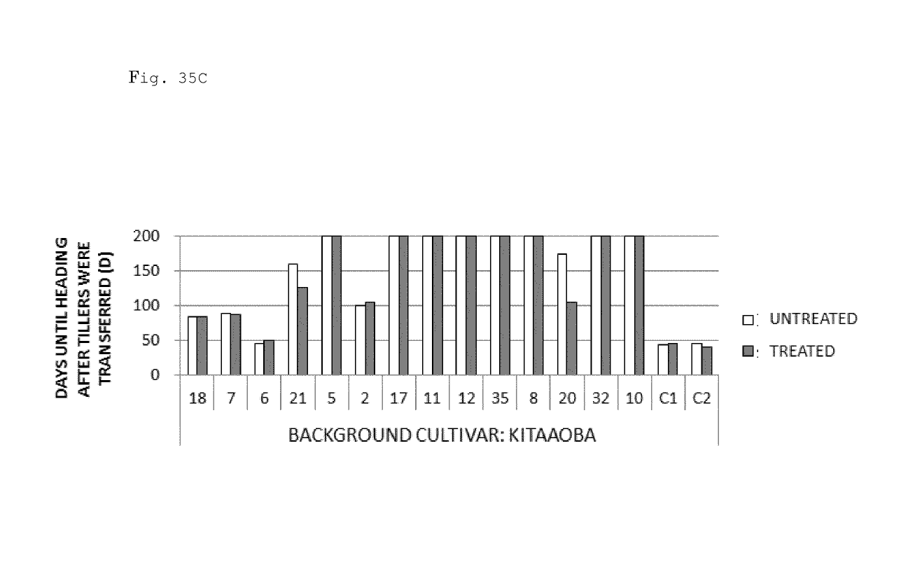

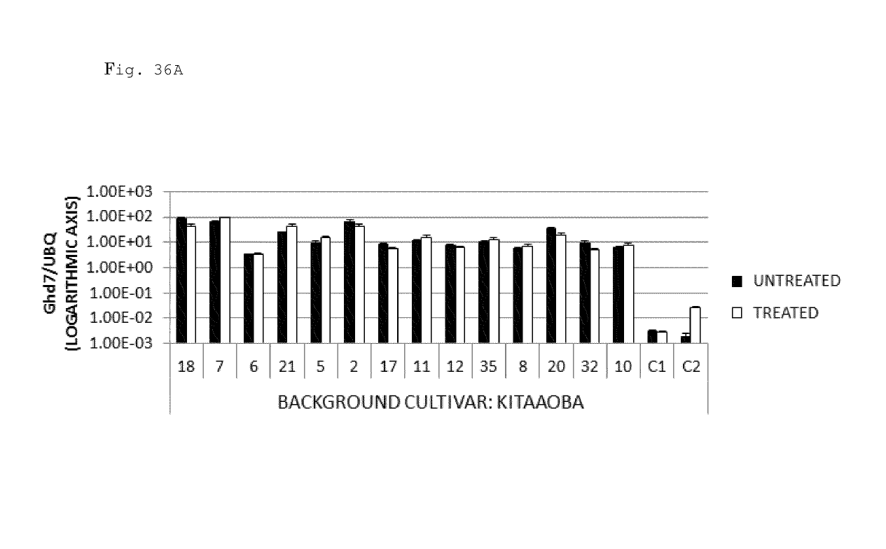

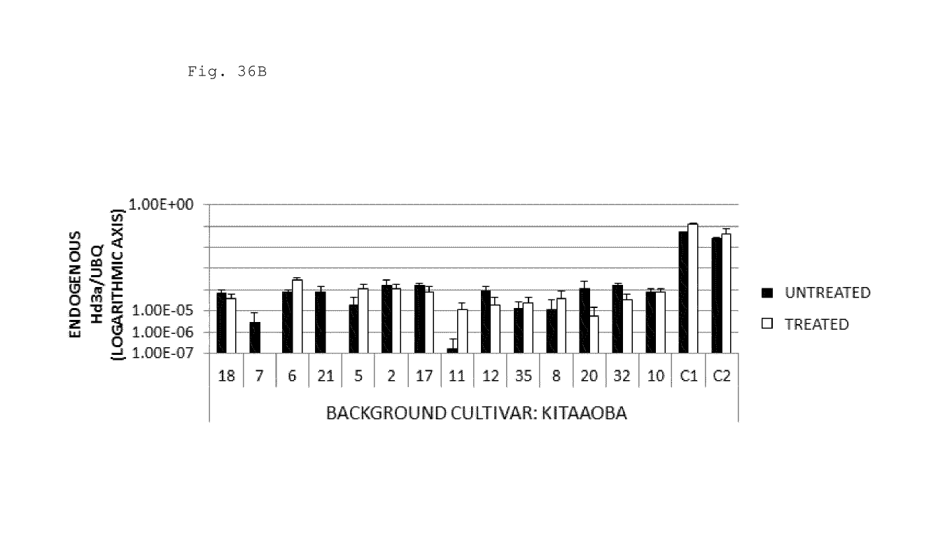

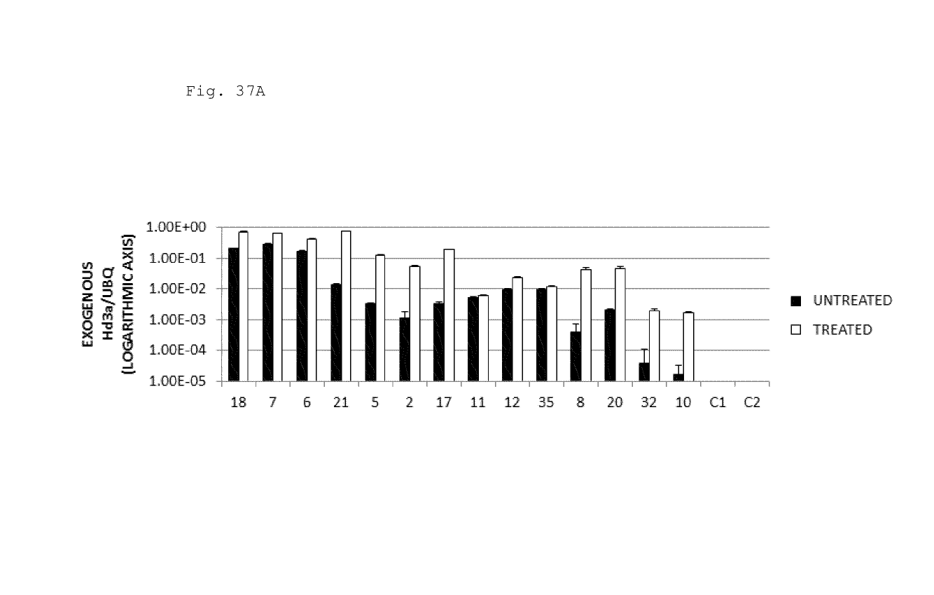

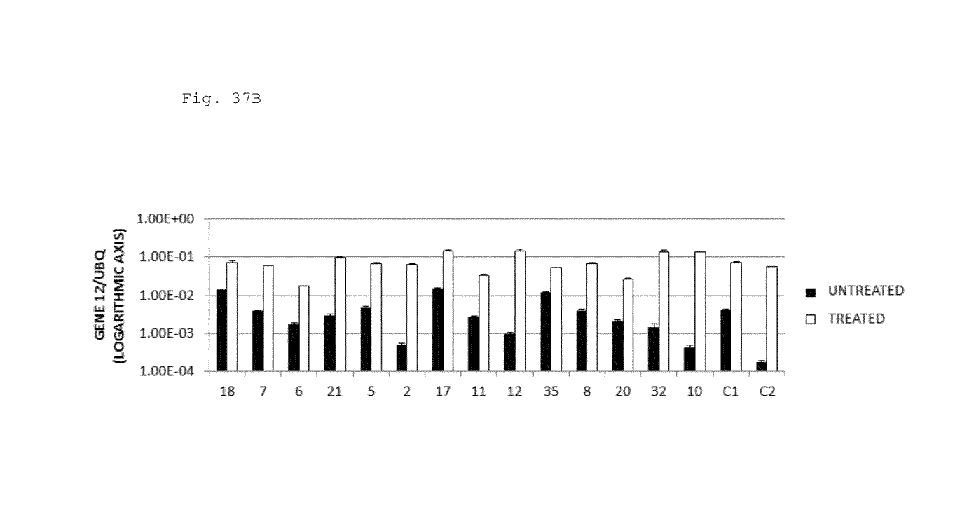

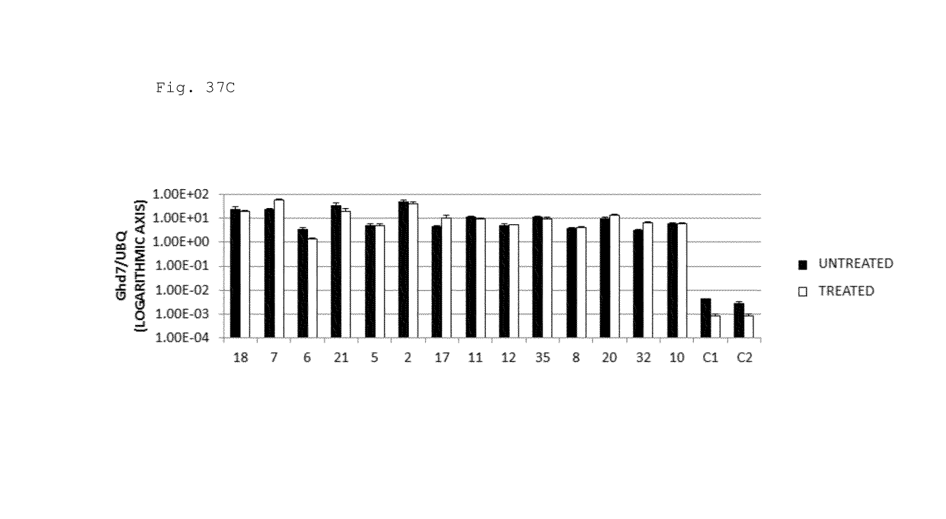

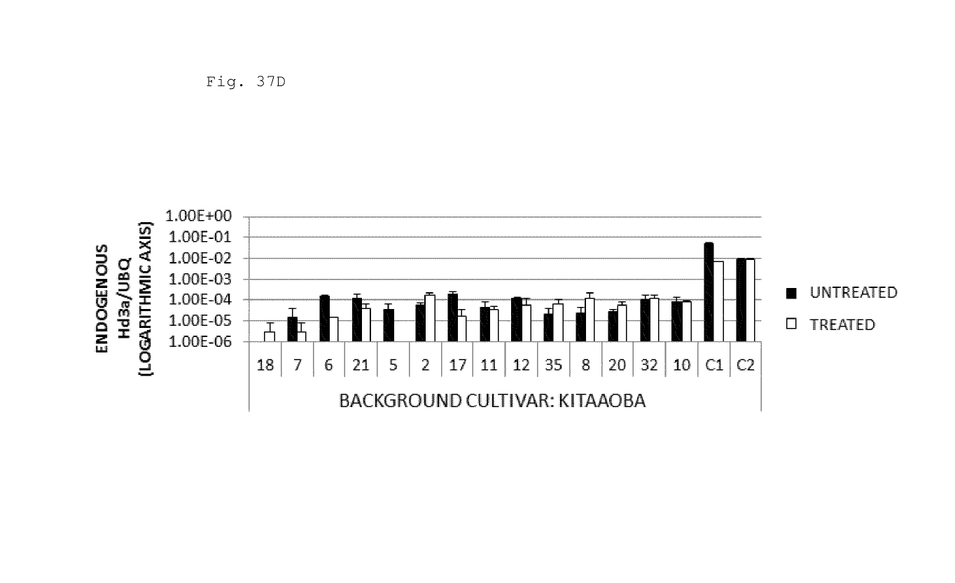

As a result of analyzing the expression in the leaves (the leaf blades on Day 3 after the chemical treatment) by the quantitative RT-PCR analysis, the induced expression of the gene (12) as a positive control and the induced expression of the exogenously introduced Hd3a gene were confirmed in many lines (FIGS. 35A and 35B). Depending on the lines, several ten-fold or more inductions were observed. Moreover, in examining the expression of the exogenously introduced Ghd7 gene together with the Hd3a gene, the constitutive expression was confirmed. It was also confirmed that the expression of the endogenous Hd3a gene was suppressed in comparison with control lines (C1 and C2) (FIGS. 36A and 36B). Further, as a result of conducting the expression analysis again using the leaf samples on Week 2 after the chemical treatment also, reproducible expression patterns were confirmed in each line (FIGS. 37A to 37D). Next, the flowering status was examined. As a result, it was observed in the #20 and #21 lines that the treated individuals produced the ears earlier than the untreated individuals. The flowering induction by the chemical treatment was confirmed (FIG. 35C).

### Example 20

### Example 2 of Applying Flowering-Time Control DNA Cassette to Feed Rice Cultivar Tachisugata

To transform the feed rice cultivar Tachisugata, the flowering-time control plasmid (pRiceFOX//Ubi:Ghd7/Gate:Adh5′UTR:Hd3a, FIG. 2B) was used in which the rice gene (12) promoter (SEQ ID NO: 1) was introduced. The transformants (T0 generation) thus produced were subjected to the flowering induction test by the plant activator treatment. Tachisugata was transformed according to the rice transformation method described in Example 1.

To handle the produced transformants in each line separately for the treatment/untreatment with the plant activator, tillers of each line were divided, transferred to a glass greenhouse, and grown until the chemical induction test was conducted. On Day 49 after the plants were transferred and grown, the individuals for the treatment were subjected to the chemical spray treatment, and the induction test was started. As the chemical, Routine 1 kg granule (Bayer CropScience AG) was used, the plants were treated every 5 days with the chemical in an amount of 0.5 g/individual at one time, hence treated three times in total. On Day 5 and Week 2 after the chemical treatment was started, leaf blades were collected from the untreated individuals and the treated individuals of each line and subjected to the quantitative RT-PCR analysis.

The RNA extraction from the leaf blade samples, the cDNA synthesis, and the quantitative RT-PCR analysis were carried out by the methods described in Example 4. In addition, Table 4 shows the sequences of the primers and the Taq Man probes used in the quantitative RT-PCR analysis.

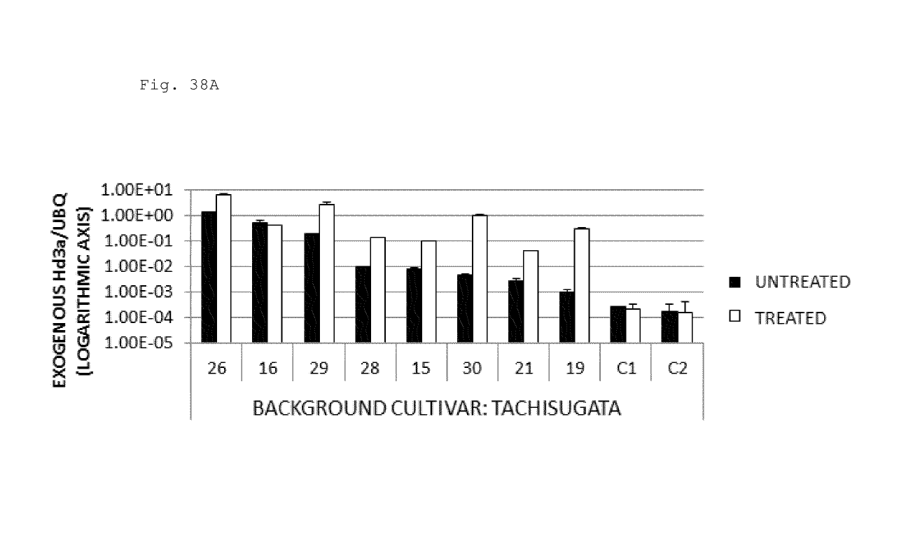

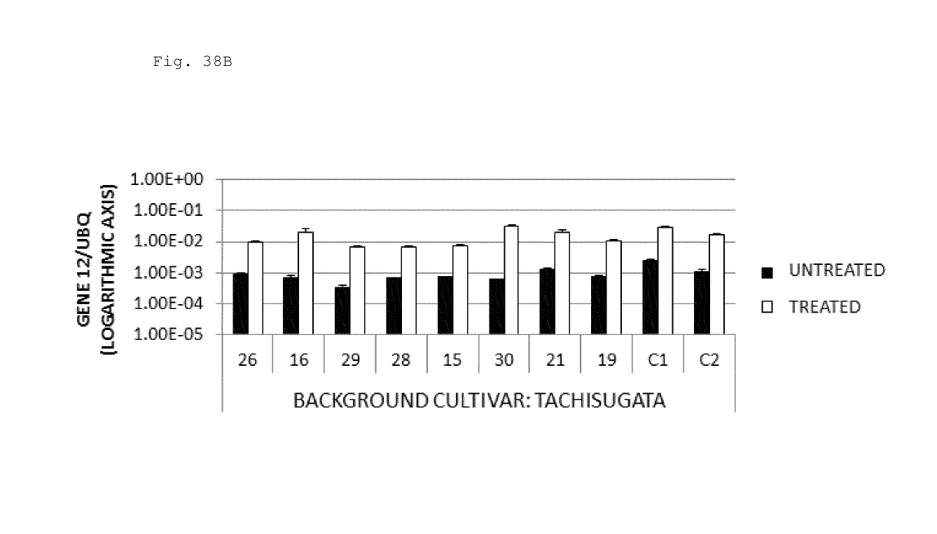

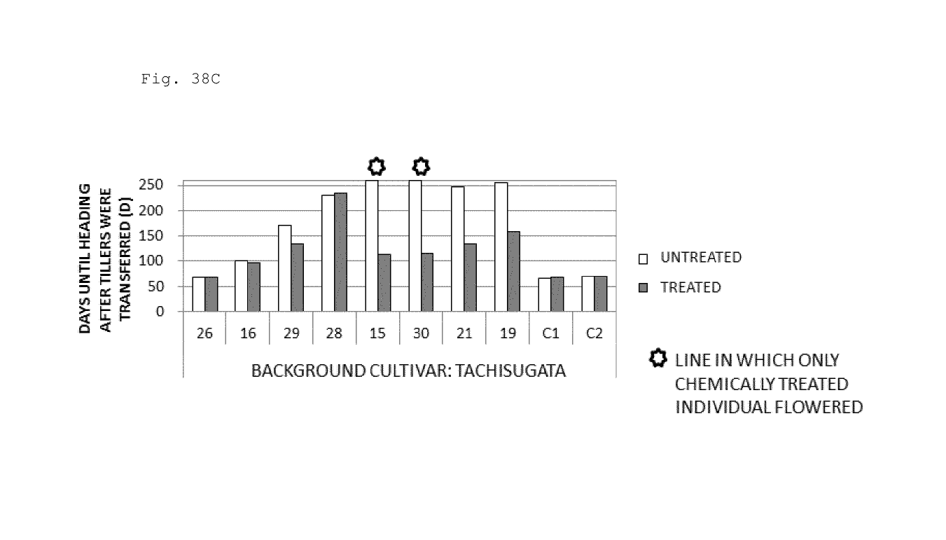

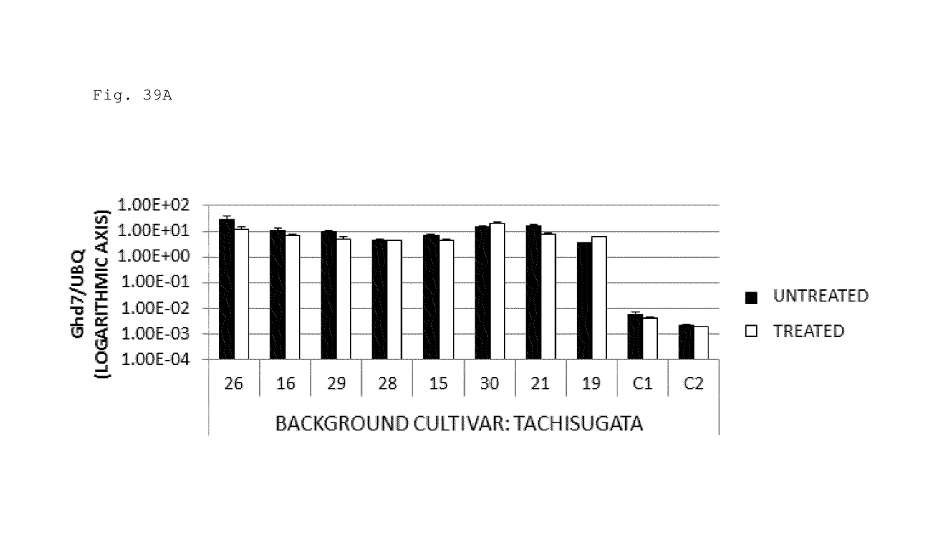

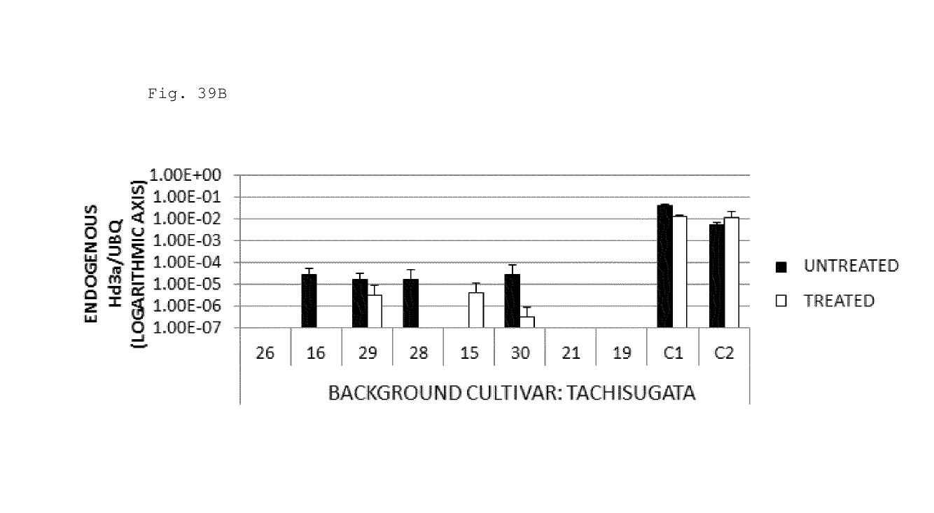

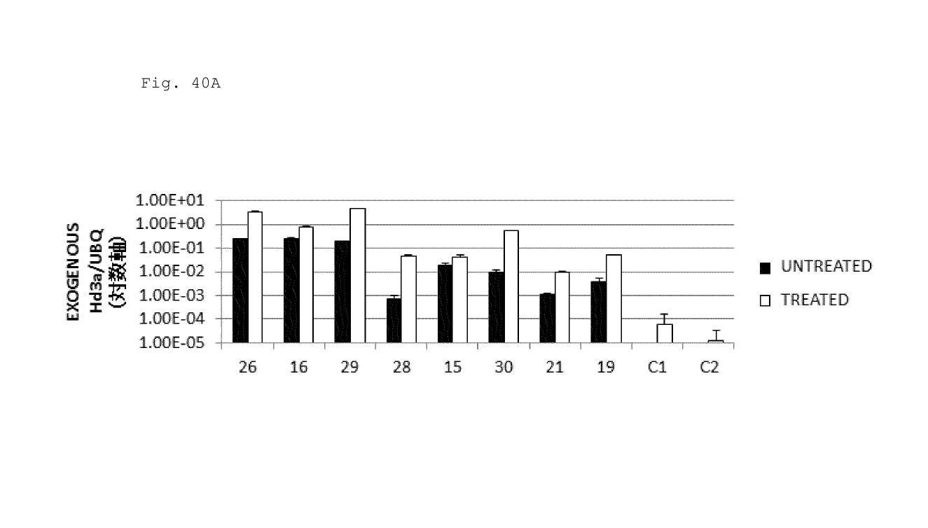

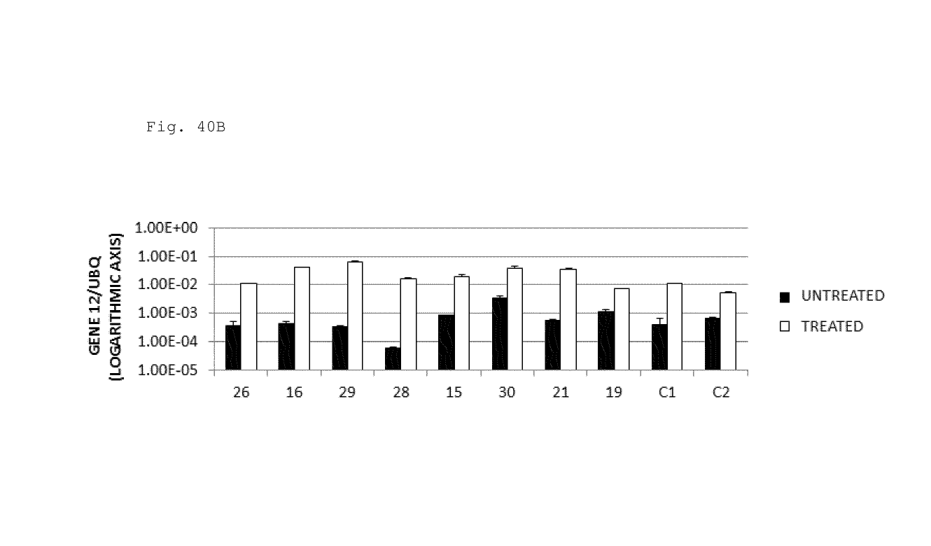

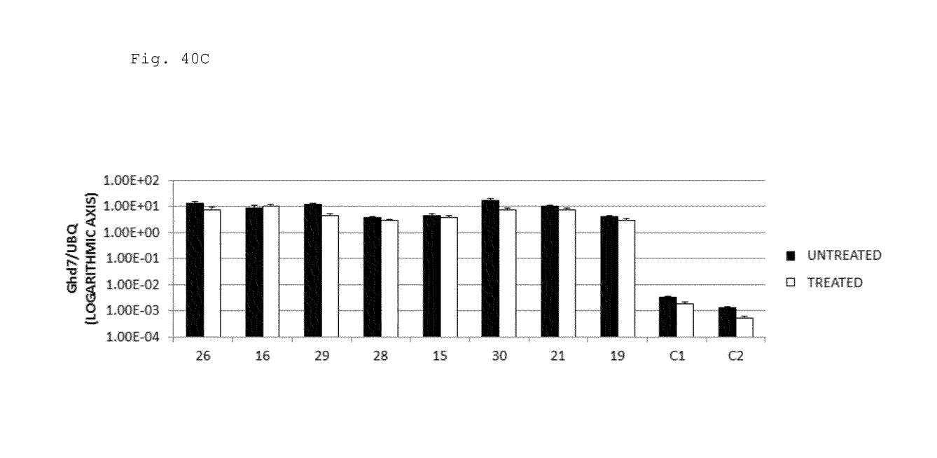

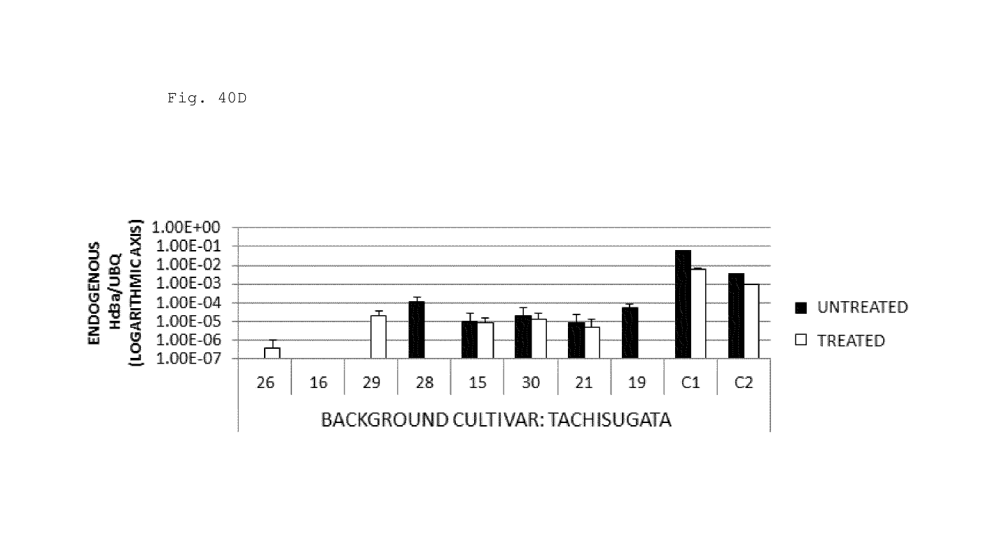

As a result of analyzing the expression in the leaves (the leaf blades on Day 5 after the chemical treatment) by the quantitative RT-PCR analysis, the expression of the exogenously introduced Hd3a gene was detected at a higher level in the treatment than in the untreatment in many lines (several ten-fold or more inductions were observed in lines having a large difference therebetween). The expression induction by the chemical was confirmed (FIG. 38A). Moreover, in the analysis on the expression of the gene (12) as a positive control of the chemical induction also, a higher level of the induced expression was confirmed in the treatment (FIG. 38B). In addition, in comparison with control lines (C1 and C2), it was confirmed that the exogenously introduced Ghd7 gene was constitutively expressed at a high level while the expression of the endogenous Hd3a gene was suppressed in the transformants (FIGS. 39A and 39B). Further, as a result of conducting the expression analysis again using the leaf samples on Week 2 after the chemical treatment also, reproducible expression patterns were confirmed in each line (FIGS. 40A to 40D). Next, the heading status was examined. As a result, it was observed in multiple lines that the treated individuals produced the ears earlier than the untreated individuals. The flowering induction by the chemical treatment was confirmed (FIG. 38C). Particularly, the ears were produced by only the individuals treated with the chemical in the #15 and #30 lines (FIG. 38C).

### INDUSTRIAL APPLICABILITY

One of important cultivation characteristics of crops is the flowering time. Heretofore, crop cultivars have been improved by targeting the yield, quality (such as taste), environmental resistance (such as disease resistance or lodging resistance), or the like, or in accordance with the usage for feed, fuel resource (such as bioethanol), or the like. However, cultivars developed so far have own flowering times based on the genetic backgrounds, and the flowering time of one cultivar is different from those of the others. This difference limits the location and timing suitable for the cultivations. For example, in rice cultivation, even if an excellent cultivar is bred in one location, it is difficult to cultivate the cultivar in other locations. Particularly, since the geography of Japan extends from north to south, it is essential to select a rice cultivar in accordance with the natural conditions such as day length at the location. Thus, in a case where the flowering time is not suitable for the location where the cultivar is to be cultivated, it is necessary to develop a new cultivar. The present invention is utilizable in expanding potential location and timing for cultivation of cultivars having different own flowering time as described above.

In consideration of the productivity of agricultural crops, the difference in the flowering time often has a great influence on the yield trait. This is presumably because of a difference in the length of the vegetative growth period; hence, an early-flowering plant flowers while small in size, and a late-flowering plant flowers while large in size, thus influencing the yields. Actually, extending the vegetative growth period increases the height and dry weight of the plant, thereby increasing the biomass. Meanwhile, there have also been reports that flowering control genes influence traits other than flowering time. For example, in rice, it is known that the Ghd7 gene not only has a function to delay the flowering time, but also influences the number of grains and the height in a field test; it has been reported that flowering control genes such as an Ehd1 gene and an Hd1 gene involved in Hd3a gene/RFT1 gene expression control act on the panicle form development (Xue et al., Nat Genet. 2008; 40 (6): 761-7, Endo-Higashi et al., Plant Cell Physiol. 2011; 52 (6): 1083-96). From the foregoing, attention has been focused on the influence of flowering control genes on traits such as yield trait other than flowering time. On the other hand, in all the crop cultivars at present, their own flowering times presumably limit the potentials to the large extents. For example, even if the photosynthetic capacity is high, a genetically early-maturing plant flowers early while small in size. As an actual example, it is known that when a rice cultivar adapted to Hokkaido is cultivated in the mainland of Japan, the plant matures much earlier, so that the yield obtained is lower than that obtained when the plant is cultivated in Hokkaido. Moreover, if a plant flowers at a different timing from the own flowering time, this may influence the quality; for example, maturing at high temperature may reduce the quality. Nevertheless, as has been described hereinabove, regarding existing agricultural cultivars, once a cultivar to be cultivated and a planting day are set, the flowering time is almost definitely determined, and the yield is also roughly determined. Thus, it is believed that the flexible flowering time regulation by the present invention can contribute to improvements in potentials, such as yield and biomass, of cultivars to be cultivated.

Among rice cultivars, some cultivars are deficient in the Ghd7 gene function for purposes of breeding or industrial applications. These cultivars have been improved mainly for the cultivations in northern areas. For this reason, when cultivated in the mainland, these cultivars flower early, and originally expected yields will not be obtained. Meanwhile, the cultivars suitably cultivated in northern areas genetically flower early, so that the flowering occurs after a limited vegetative growth period. To overcome this shortcoming, such cultivars are bred to be high-yield cultivars in many cases. Thus, in mid-latitude or southern areas, the present invention is conceivably applied to cultivars which cannot exhibit their potentials due to the flowering time incompatibility caused by the Ghd7 gene function deficiency.

Moreover, it is known that plants synthesize sugars by photosynthesis in leaves during daytime, and accumulate the sugars in the form of starch in the leaves or translocate the sugars in the form of sucrose to other organs. In rice, translocated sucrose is accumulated in the form of starch at the base of a leaf (culm). However, when flower buds are induced, the starch accumulated in the leaf and so forth is translocated in the form of sucrose for the growth of rice kernels. Thus, it is conceivable that the accumulations of sugars in stems and leaves can be controlled through the flowering-time control. From the foregoing, the present invention is applicable also to rice cultivars for feeds such as WCS (whole crop silage) obtained by cutting all the above-the-ground parts including stem and leaf parts for feed.

Conceivably, applying the present invention to cultivars of other Poaceae monocot crops such as sugarcane and corn can also greatly change the bioethanol production efficiency.

### SEQUENCE LISTING FREE TEXT

### SEQ ID NO: 1

<223> promoter of the gene 12 (Oryza sativa)

### SEQ ID NO: 2

<223> Hd3a cDNA (Kasalath)

### SEQ ID NO: 4

<223> Hd3a cDNA (Nippon-bare)

### SEQ ID NO: 6

<223> Ghd7 cDNA

### SEQ ID NOs: 8 to 18

<223> Artificially synthesized primer sequence

### SEQ ID NO: 19

<223> pRiceFOX/Ubi:Ghd7/Gate:Hd3a

### SEQ ID NO: 20

<223> pRiceFOX/Ubi:Ghd7/Gate:Adh5′UTR:Hd3a

### SEQ ID NOs: 21 to 34

<223> Artificially synthesized primer sequence

### SEQ ID NO: 35

<223> promoter of the gene 1

### SEQ ID NO: 36

<223> promoter of the gene 2

### SEQ ID NO: 37

<223> promoter of the gene 3

### SEQ ID NO: 38

<223> promoter of the gene 4

### SEQ ID NO: 39

<223> promoter of the gene 5

### SEQ ID NO: 40

<223> promoter of the gene 6

### SEQ ID NO: 41

<223> promoter of the gene 7

### SEQ ID NO: 42

<223> promoter of the gene 8

### SEQ ID NO: 43

<223> promoter of the gene 9

### SEQ ID NO: 44

<223> promoter of the gene 10

### SEQ ID NO: 45

<223> promoter of the gene 11

### SEQ ID NO: 46

<223> promoter of the gene 13

### SEQ ID NOs: 47 to 74

<223> Artificially synthesized primer sequence

### SEQ ID NO: 75

<223>3′UTR of the gene 3

### SEQ ID NOs: 76 to 132

<223> Artificially synthesized primer sequence

### SEQ ID NO: 133

<223> promoter 1 of the gene 12 (Zea mays)

### SEQ ID NOs: 134 to 136

<223> Artificially synthesized primer sequence

### SEQ ID NO: 137

<223> promoter 2 of the gene 12 (Zea mays)

### SEQ ID NOs: 138 and 139

<223> Artificially synthesized primer sequence

### SEQ ID NO: 140

<223> pKLB525/Ubi:Ghd7/Gate:Hd3a

### SEQ ID NO: 141

<223> pKLB525/Ubi:Ghd7/Gate:Adh5rUTR:Hd3a

### SEQ ID NO: 142

<223> Artificially synthesized primer sequence

1. A Poaceae plant whose flowering time is controllable by a plant activator treatment, the Poaceae plant comprising an expression construct in which an Hd3a gene is ligated downstream of a promoter sensitive to a plant activator.

2. The Poaceae plant according to claim 1, wherein the plant activator is any one of probenazole and isotianil.

3. The Poaceae plant according to claim 2, wherein the promoter is a DNA of any one of (a) to (c) below:
(a) a DNA having a nucleotide sequence of any one of SEQ ID NOs: 1, 133, and 137;
(b) a DNA having a nucleotide sequence of any one of SEQ ID NOs: 1, 133, and 137 in which one or more nucleotides are substituted, deleted, added, and/or inserted, the DNA having an activity of the promoter sensitive to the plant activator; and
(c) a DNA having a nucleotide sequence having a homology of 70% or more with the nucleotide sequence of any one of SEQ ID NOs: 1, 133, and 137, the DNA having the activity of the promoter sensitive to the plant activator.

4. The Poaceae plant according to claim 1, further comprising an expression construct of a gene encoding a protein that suppresses an expression of an endogenous Hd3a gene but does not suppress an activity of an Hd3a protein.

5. The Poaceae plant according to claim 4, wherein the protein that suppresses the expression of the endogenous Hd3a gene but does not suppress the activity of the Hd3a protein is a Ghd7 protein.

6. The Poaceae plant according to claim 4, wherein the gene encoding the protein that suppresses the expression of the endogenous Hd3a gene but does not suppress the activity of the Hd3a protein is ligated downstream of a constitutive expression promoter.

7. The Poaceae plant according to claim 6, wherein the constitutive expression promoter is a corn-derived ubiquitin promoter.

8. A Poaceae plant, which is any one of a progeny and a clone of the Poaceae plant according to claim 1.

9. A propagation material of the Poaceae plant according to claim 1.

10. A method for producing a Poaceae plant whose flowering time is controllable by a plant activator treatment, the method comprising the step of introducing into a Poaceae plant cell an expression construct in which an Hd3a gene is ligated downstream of a promoter sensitive to a plant activator, and regenerating the plant.

11. A method for inducing flowering of a Poaceae plant, the method comprising the step of treating the Poaceae plant according to claim 1 with the plant activator.

12. An agent for inducing flowering of the Poaceae plant according to claim 1, the agent comprising the plant activator as an active ingredient.
